SEQUENCE LISTING (4 sequences)

SEQ ID NO: 1 - RNA, 20 nt, synthetic construct
  misc_feature 1..20: note = Description of sequence: sgRNA: sgRNA-1
  source 1..20: mol_type = other RNA
ttatggacagaattaagaag

SEQ ID NO: 2 - RNA, 20 nt, synthetic construct
  misc_feature 1..20: note = Description of sequence: sgRNA: sgRNA-2
  source 1..20: mol_type = other RNA
ctgtccataagatttgaaag

SEQ ID NO: 3 - RNA, 20 nt, synthetic construct
  misc_feature 1..20: note = Description of sequence: sgRNA: sgRNA-3
  source 1..20: mol_type = other RNA
aattttgttctggtagccat

SEQ ID NO: 4 - RNA, 20 nt, synthetic construct
  misc_feature 1..20: note = Description of sequence: sgRNA: sgRNA-4
  source 1..20: mol_type = other RNA
ggtatttcagctagtgccta

(19) United States
(12) Patent Application Publication
(10) Pub. No.: US 2024/0376452 A1
(43) Pub. Date: Nov. 14, 2024

(54) COMPOSITIONS AND METHODS FOR TREATING ALZHEIMER'S DISEASE

(71) Applicant: The Hong Kong University of Science and Technology, Kowloon (CN)

(72) Inventors: Nancy Yuk Yu Ip, Hong Kong (CN); Kit Yu Fu, Kowloon, Hong Kong SAR (CN); Fanny Chui Fun Ip, Hong Kong (CN); Yuanbing Jiang, Chengdu, Sichuan (CN); Xiaopu Zhou, Chongqing (CN); Li Ouyang, Hong Kong (CN); Hiu Yi Wong, Hong Kong (CN); Yangyang Duan, Kunming (CN)

(21) Appl. No.: 18/577,072

(22) PCT Filed: Jul. 27, 2022
(86) PCT No.: PCT/CN2022/108136

§ 371 Date: Jan. 5, 2024

Related U.S. Application Data
(60) Provisional application No. 63/226,165, filed on Jul. 27, 2021.

(51) Int. Cl. C12N 9/22; A61K 31/7105; A61K 48/00; A61P 25/28; C12N 15/113

(52) U.S. Cl. CPC C12N 9/22; A61K 31/7105; A61K 48/005; A61P 25/28; C12N 15/1131; C12N 2310/11; C12N 2310/20

(57) ABSTRACT

Provided are new compositions and methods useful for the treatment, prevention, and potential cure of Alzheimer's Disease by disrupting the genomic locus of IL1LR1 gene including rs1921622 and/or other 574 sST2-associated genomic sites and/or 3′-UTR of sST2 gene/transcript.

### RELATED APPLICATIONS

This application is a U.S. National Stage of PCT/CN2022/108136, International Filing Date Jul. 27, 2022, which claims priority to U.S. Provisional Patent Application No. 63/226,165, filed Jul. 27, 2021, the contents of all of which are hereby incorporated by reference in the entirety for all purposes.

### SEQUENCE LISTING

The instant application contains a Sequence Listing which has been submitted electronically in XML file format and is hereby incorporated by reference in its entirety. Said XML copy, created on Sep. 21, 2023 is named 091256-1408282-002510US_SL.xml and is 9,149 bytes in size.

### BACKGROUND OF THE INVENTION

Brain diseases such as neurodegenerative diseases and neuroinflammatory disorders are devastating conditions that affect a large subset of the population. Many of such diseases are incurable, highly debilitating, and often result in progressive deterioration of the brain structure and function over time. Disease prevalence is also increasing rapidly due to growing aging populations worldwide, since the elderly are at high risk for developing these conditions. Currently, many neurodegenerative diseases and neuroinflammatory disorders are difficult to diagnose due to limited understanding of the pathophysiology of these diseases. Meanwhile, current treatments are ineffective and do not meet market demand; demand that is significantly increasing each year due to the ever-growing aging populations. For example, Alzheimer's disease (AD) is marked by gradual but progressive decline in learning and memory, and a leading cause of mortality in the elderly. Increasing prevalence of AD is driving the need and demand for better and earlier diagnostics. According to Alzheimer's Disease International, the disease currently affects 46.8 million people globally, but the number of cases is projected to triple in the coming three decades. One of the countries with the fastest elderly population growth is China. Based on population projections, by 2030 one in four individuals will be over the age of 60, which will place a vast proportion at risk of developing AD. In fact, the number of AD cases in China doubled from 3.7 million to 9.2 million from 1990-2010, and the country is projected to have 22.5 million cases by 2050. Hong Kong's population is also aging quickly. It is estimated that the elderly aged 65+ will make up 24% of the population by 2025, and 39.3% of the population by 2050. The number of AD cases is projected to rise to 332,688 by 2039. As such, there exists an urgent need for developing new methods for effectively treating AD patients who suffer from this devastating condition. This invention fulfills this and other related needs by disclosing novel compositions and methods useful for effective treatment, and potentially providing a cure, of the disease.

### BRIEF SUMMARY OF THE INVENTION

The application provides the first disclosure of genetic loci, including rs1921622 and/or other 574 sST2-associated genetic variants, and/or three-prime (3′)-untranslated regions of sST2, upon being disrupted leading to diminished expression of soluble ST2 (sST2) protein, as a target for therapeutic intervention of Alzheimer's Disease through genetic manipulation. New compositions and methods for treating Alzheimer's Disease by way of suppression or elimination of the effects of genomic sequence encompassing rs1921622, other 574 sST2-associated genetic variants, or the three-prime (3′)-untranslated regions of sST2 are therefore devised from this discovery.

As such, in a first aspect, this invention provides a method for treating Alzheimer's Disease in a person or reducing the person's risk for later developing Alzheimer's Disease. The claimed method comprises the step of administering to the person an effective amount of a composition disrupting a genomic sequence encompassing (i) rs1921622, and/or (ii) any one or more of other 574 sST2-associated genetic variants listed in Table 4 and Table 5, and/or (iii) 3′-untranslated region (UTR) of sST2 gene coding sequence or transcript.

In some embodiments, the claimed method comprises, prior to the administering step, sequencing at least a portion of the person's genome. In some embodiments, the person is deemed an APOE-ε4 carrier, either a heterozygote or homozygote carrier. In other cases, the person is a non-APOE-ε4 carrier. In some cases, the person is a female. In some cases, the person is a male. In some embodiments, the person has an A allele at rs1921622. In some embodiments, the person has at least one allele at a specified genetic locus shown in Table 4. In some embodiments, the person has been diagnosed with AD. In some embodiments, the person is not yet diagnosed with AD but has known risk factors for AD such as family history of AD or carrying one or more genetic alleles known to increase AD risk, e.g., as a female APOE-ε4 carrier. In some embodiments, the genomic sequence encompassing rs1921622 (or another genetic locus named in Table 4 or 5) comprises sequence about 300 basepairs upstream and downstream from rs1921622 (or another genetic locus in Table 4 or 5), preferably about 250, 200, 150, 100, 50, 30, or 20 basepairs upstream and downstream from rs1921622 (or another genetic locus in Table 4 or 5). In some embodiments, the composition comprising an siRNA, a microRNA, a miniRNA, a lncRNA, or an antisense oligonucleotide targeting the genomic sequence encompassing rs1921622 (or another genetic locus in Table 4 or 5) or the 3′-UTR of the sST2 gene. In some embodiments, the composition comprises one or more vectors encoding an endonuclease guided by a small guide RNA (sgRNA) and two sgRNAs targeting two locations within the genomic sequence encompassing rs1921622 (or another genetic locus listed in Table 4 or 5) or the 3′-UTR of the sST2 gene. In some embodiments, the composition comprises one vector encoding a Cas9 nuclease and two sgRNAs. In some embodiments, the one or more vectors are one or more viral vectors. In some embodiments, the composition is administered by subcutaneous, intramuscular, intravenous, intraperitoneal, or intracranial injection or by oral or nasal administration. In some embodiments, the composition is administered in the form of a solution, a suspension, a powder, a paste, a tablet, or a capsule.

In a second aspect, the present invention provides a composition comprising an effective amount of one or more agents that disrupt a genomic sequence encompassing (i) rs1921622, and/or (ii) any one or more of other 574 sST2-associated genetic variants listed in Table 4 and Table 5, and/or (iii) 3′-untranslated region (UTR) of sST2 gene coding sequence or transcript, plus one or more physiologically acceptable excipient.

In some embodiments, the genomic sequence encompassing rs1921622 (or another genetic locus named in Table 4 or 5) comprises sequence about 300 bps upstream and downstream from rs1921622 (or another genetic locus in Table 4 or 5), preferably about 250, 200, 150, 100, 50, 30, or 20 bps upstream and downstream from rs1921622 (or another genetic locus in Table 4 or 5). In some embodiments, the composition comprising an siRNA, a microRNA, a miniRNA, a lncRNA, or an antisense oligonucleotide targeting the genomic sequence encompassing rs1921622 (or another genetic locus in Table 4 or 5) or the 3′-UTR of the sST2 gene. In some embodiments, the composition comprises one or more vectors encoding an endonuclease guided by a small guide RNA (sgRNA) and two sgRNAs targeting two locations within the genomic sequence encompassing rs1921622 (or another genetic locus listed in Table 4 or 5) or the 3′-UTR of the sST2 gene. In some embodiments, the composition comprises one vector encoding a Cas9 nuclease and two sgRNAs. In some embodiments, the one or more vectors are one or more viral vectors. In some embodiments, the composition is administered by subcutaneous, intramuscular, intravenous, intraperitoneal, or intracranial injection or by oral or nasal administration. In some embodiments, the composition is administered in the form of a solution, a suspension, a powder, a paste, a tablet, or a capsule.

In a third aspect, the present invention provides a kit for treating Alzheimer's Disease in a person or for reducing the person's risk of later developing Alzheimer's Disease. The kit comprises a first container containing a composition disrupting a genomic sequence encompassing rs1921622 (or another genetic locus in Table 4 or 5) or the 3′-UTR of the sST2 gene.

In some embodiments, the composition is formulated for subcutaneous, intramuscular, intravenous, intraperitoneal, or intracranial injection, or for oral or nasal administration. In some embodiments, the composition comprising an siRNA, a microRNA, a miniRNA, a lncRNA, or an antisense oligonucleotide targeting the genomic sequence encompassing rs1921622 (or another genetic locus in Table 4 or 5) or the 3′-UTR of the sST2 gene. In some embodiments, the composition comprises (1) one or more vectors encoding an endonuclease guided by a small guide RNA (sgRNA) and (2) two sgRNAs targeting two locations within the genomic sequence encompassing rs1921622 (or another genetic locus in Table 4 or 5) or the 3′-UTR of the sST2 gene. In some embodiments, the kit contains a composition comprising one vector encoding a Cas9 nuclease and two sgRNAs. In some embodiments, the kit further contains a second container containing agents for sequencing at least a portion of the person's genome (e.g., the APOE-ε4 gene). Optionally, the kit also includes an instruction manual for administration of the composition.

Related to this aspect of the present invention, a use of one or more agents disrupting a genomic sequence encompassing rs1921622, and/or other 574 sST2-associated genetic variants, and/or 3′-untranslated regions of sST2 is further provided in accordance with the disclosure herein for the manufacturing of (1) a medicament for treating Alzheimer's Disease; and/or (2) a kit containing the medicament for treating Alzheimer's Disease.

### BRIEF DESCRIPTION OF THE DRAWINGS

FIGS. 1A and 1B. Diagrams illustrating IL-33/ST2 signaling. (FIG. 1A) Schematic showing the soluble ST2 (sST2) and fill-length ST2 (ST2L) transcripts. (FIG. 1B) The IL-33/ST2 signaling pathways. IL-33, interleukin 33.

FIGS. 2A to 2H. Circulating sST2 levels are associated with Alzheimer's disease and its pathologic changes. (FIG. 2A) Individual plasma soluble ST2 (sST2) levels stratified according to disease phenotype (n=336 healthy controls [HCs], n=277 patients with AD from the Chinese_cohort_1; linear regression test, T=3.241, **P<0.01). Data are presented as box-and-whisker plots including maximum, 75^{th} percentile, median, 25^{th} percentile, and minimum values; plus signs (+) denote corresponding mean values. (FIG. 2B) Correlation between plasma sST2 level and intracranial volume (ICV)-normalized gray matter volume (n=192 participants from the Chinese_cohort_1); r, Pearson's correlation coefficient. (FIG. 2C) Correlation between plasma sST2 and P-tau181 levels (n=271 participants from the Chinese_cohort_1). (FIG. 2D) Correlation between plasma sST2 and NfL (neurofilament light polypeptide) levels (n=251 participants from the Chinese_cohort_1). (FIG. 2E) Correlation between cerebrospinal fluid (CSF) and plasma levels of sST2 (n=107 participants from the Stanford Alzheimer's Disease Research Center [ADRC] cohort). (FIG. 2F) Individual CSF sST2 levels stratified according to disease phenotype (n=11 HCs, n=75 patients with AD from the UKBBN cohort; linear regression test, T=3.355, **P<0.01). (FIG. 2G, FIG. 2H) Correlations between amyloid-beta (Aβ) staining in the postmortem frontal cortex and CSF sST2 level in patients with AD (n=51 participants from the UKBBN cohort). Participants were stratified into 2 groups according to CSF sST2 level: low, ≤3.6 ng/mL; high, >3.6 ng/mL. The vertical dashed line in (h) indicates the CSF sST2 level (3.6 ng/mL) with the largest Youden's index value for distinguishing HCs from patients with AD. Representative images of Aβ staining in patients with AD with low and high CSF sST2 levels (g) and results of correlation analysis (h). Scale bar, 100 μm.

FIGS. 3A to 3E. Plasma sST2 level was elevated in patients with Alzheimer's disease without a history of cardiovascular diseases in the Chinese_cohort_1. (FIG. 3A) Individual plasma soluble ST2 (sST2) levels stratified by cardiovascular disease (CVD) phenotype (n=97 cognitively normal participants with no history of CVDs [non-CVD], n=30 cognitively normal participants with heart disease [HD], n=201 cognitively normal participants with hypertension [HT], n=72 cognitively normal participants with diabetes mellitus [DM], n=98 cognitively normal participants with hyperlipidemia [HL] from the Chinese_cohort_1, respectively; linear regression test, T=2.206, 0.296, −1.493, and −0.461 for tests on heart disease, hypertension, diabetes mellitus and hyperlipidemia, respectively; *P<0.05). Data are presented as box-and-whisker plots including maximum, 75^{th} percentile, median, 25^{th} percentile, and minimum values; plus signs (+) denote corresponding mean values. (b-e) Plasma sST2 level is associated with Alzheimer's disease (AD) and associated endophenotypes. (FIG. 3B) Individual plasma sST2 levels stratified by disease phenotype (n=97 healthy controls [HCs], n=70 patients with AD from the Chinese_cohort_1 without a history of CVDs [non-CVD cohort], respectively; linear regression test, T=3.151, **P<0.01). (c-e) Correlations between plasma sST2 levels and endophenotypes in patients with AD. (FIG. 3C) Gray matter volume normalized to intracranial volume (ICV) (n=48 participants from the non-CVD cohort). (FIG. 3D) Plasma P-tau181 level (n=77 participants from the non-CVD cohort). (FIG. 3E) Plasma NfL (neurofilament light polypeptide) level (n=60 participants from the non-CVD cohort). r, Pearson's correlation coefficient.

FIG. 4A to 4I. Elevation of brain sST2 levels exacerbates AP accumulation and impairs microglial Aβ-clearance capacity. (FIG. 4A) Schematic of intracerebroventricular (i.e.v.) delivery of soluble ST2 (sST2) to 3-month-old 5XFAD mice. (b-e) Amyloid-beta (Aβ) deposition in the cortices of 4-month-old 5XFAD mice after i.e.v. delivery of sST2-Fc or Fc as a control. (b, c) DAB staining of Aβ. (FIG. 4B) Representative images; scale bar, 1 mm. (FIG. 4C) Quantification of Aβ plaques (% of total cortical area) (control: n=6 mice, sST2: n=7 mice; unpaired Student's t-test, T=2.758, *P<0.05). Data in bar charts are mean+SEM. (FIG. 4D) Confocal images of X-34-stained AD deposits (blue) and 4G8-labeled Aβ (red). Filamentous (filled arrowheads) and compact (hollow arrowheads) Aβ plaques are labeled. Scale bar, 100 μm. (FIG. 4E) Quantification of filamentous, compact, and inert Aβ plaques (control: n=6 mice, sST2: n=7 mice; unpaired Student's t-test, T=3.325, 2.274, and −0.770, respectively; *P<0.05, **P<0.01). (f, g) Colocalization of microglia and Aβ plaques in the cortices of 4-month-old 5XFAD mice after i.e.v. delivery of sST2-Fc or Fc as a control. (FIG. 4F) Representative images showing co-staining of 4G8-labeled Aβ plaques (white) and Iba-1⁺ microglia (green; red arrowheads indicate microglial soma) using merged confocal Z-stack images with orthogonal X-Z and Y-Z views. Scale bar, 10 μm. (FIG. 4G) Quantification of microglial coverage of Aβ plaques (control: n=6 mice, sST2: n=7 mice; unpaired Student's t-test, T=−2.298; *P<0.05). (h, i) Microglial Aβ uptake activity in the cortices of 4-month-old 5XFAD mice after i.e.v. delivery of sST2-Fc or Fc as a control. Representative scatterplot (FIG. 4H) and quantification (FIG. 4I) show the percentages of CD11Ib⁺ cells containing methoxy-X04-labeled Aβ (control: n=7 mice, sST2: n=7 mice; unpaired Student's t-test,T=−3.620, **P<0.01). The scatterplots of wild-type (WT) mice in (FIG. 4H) were used to gate methoxy-X04⁺ microglia (i.e.. MeX04⁺CD11b⁺ cells).

FIG. 5. Representative images of filamentous, compact, and inert Aβ plaques in 5XFAD mice. Confocal images of X-34-stained amyloid-beta (Aβ) deposits (blue) and 4G8-labeled Aβ (red) in the cortices of 4-month-old 5XFAD mice. Scale bar, 10 μm.

FIGS. 6A to 6C. Elevated brain sST2 leads to increased number of microglia in 5XFAD mice. (a, b) Brain soluble ST2 (sST2) increases the number of microglia in 5XFAD mice. Representative images (FIG. 6A) and quantification (FIG. 6B) of Iba-1⁺ microglia in the cortices of 4-month-old 5XFAD mice after intracerebroventricular delivery of sST2-Fc or Fc as a control (control: n=6 mice, sST2: n=7 mice; unpaired Student's t-test, T=4.491, ***P<0.001). Scale bar, 50 μm. Data in bar charts are mean+SEM. (FIG. 6C) Quantification of microglia around small (i.e., radius≤8 μm) and large (i.e., radius>8 μm) amyloid-beta (Aβ) plaques (control: n=6 mice, sST2: n=7 mice; unpaired Student's t-test, T=2.661 and 2.551, respectively, *P<0.05).

FIG. 7. The gating strategy for amyloid-beta (Aβ)⁺ microglia. Cells were gated on forward (FSC=size) and sideward scatter (SSC=internal structure). We used FSC and trigger pulse width to discriminate single cells from cell doublets or aggregates. We used unstained controls to identify CD11b⁺ cell populations. We used samples from C57BL6J mice to identify methoxy-X04⁺ cell populations.

FIGS. 8A to 8F. Effects of sex and age on sST2 levels. (FIG. 8A) Individual plasma soluble ST2 (sST2) levels stratified by sex and disease phenotype (n=132 male healthy controls [HCs], n=84 male patients with Alzheimer's disease [AD], n=204 female HCs, n=193 female patients with AD from the Chinese_cohort_1, respectively; linear regression test, test for effects of sex: T=−4.918 and −2.407 in HCs and patients with AD, respectively, #P<0.05, ###P<0.001; test for effects of AD: T=1.534 and 3.008 in males and females, respectively, **P<0.01). Data are presented as box-and-whisker plots including maximum, 75^{th} percentile, median, 25^{th} percentile, and minimum values; plus signs (+) denote corresponding mean values. (FIG. 8B) Correlations between plasma sST2 levels and age in males and females (n=216 and 397 males and females in the Chinese_cohort_1, respectively; males: Pearson's r²=0.0010, P=0.6410; females: Pearson's r²=0.0366, P<0.0001). (FIG. 8C) Correlations between plasma sST2 level and age in males and females from the INTERVAL and LonGenity cohorts² (n=1,685 and 1,616 males and females from the INTERVAL cohort, n=432 and 530 males and females from the LonGenity cohort, respectively; correlation test in the INTERVAL cohort [age 18-76 years], males: Pearson's r^{z}=0.0169, P<0.0001; females: Pearson's r²=0.0001, P=0.7715; correlation test in the LonGenity cohort [age 65-94 years], males: Pearson's r²=0.0100, P=0.0093; females: Pearson's r²=0.0196, P=0.0001). (FIG. 8D) Correlations between cerebrospinal fluid (CSF) sST2 level and age in males and females from the Japanese cohort (n=68 and 65 males and females, respectively; males: Pearson's r²=0.0441, P=0.0812; females: Pearson's r²=0.1521, P=0.0014). (e, f) Contributions of age and sex to the variance of sST2 levels. Numbers denote the proportions of plasma sST2 (FIG. 8E) and CSF sST2 (FIG. 8F) variance explained by age, sex and, other factors in the Chinese_cohort_1 and Japanese cohort, respectively.

FIGS. 9A to 9D. The rs1921622 A allele is associated with a lower circulating sST2 level. (FIG. 9A) Manhattan plot showing genetic variants at the IL1RL1 locus that are associated with plasma soluble ST2 (sST2) level, as identified by a genome-wide association study of plasma sST2 levels in the Chinese_cohort_1. Horizontal lines indicate the suggestive threshold (P=1E-5, blue) and genome-wide threshold (P=5E-8, red). (FIG. 9B) Regional association plot of genetic variants at the IL1RL1 locus and plasma sST2 level. The purple diamond indicates the sentinel variant rs1921622. The color scale indicates the linkage disequilibrium (measured as r²) between rs1921622 and its neighboring variants. (c, d) Plasma (FIG. 9C) and cerebrospinal fluid (CSF) (FIG. 9D) sST2 levels in individuals stratified according to rs1921622 genotype (plasma sST2 level:n=107, 206, and 114 G/G, G/A, and A/A carriers from the Chinese_cohort_1, respectively; linear regression test, T=−11.207, ***P<0.001; CSF sST2 level: n=20, 44, and 22 G/G, G/A, and A/A carriers from the UKBBN cohort, respectively; linear regression test, T=−2.615, *P<0.05). Data in box-and-whisker plots include maximum, 75^{th} percentile, median, 25^{th} percentile, and minimum values; plus signs (+) denote corresponding mean values.

FIGS. 10A to 10C. Fine-mapping analysis of sST2-associated genetic variants in the IL1RL1 gene. (FIG. 10A) Quantile-quantile (Q-Q) plot showing the p-value distribution of the genome-wide association study results. The genomic inflation factor (A) is shown. (FIG. 10B) Haplotype analysis of the 79 genetic variants linked with rs1921622 (r²>0.7). Each numbered column represents 1 of the 79 variants; red and blue indicate the minor and major alleles, respectively. Each row represents a particular haplotype defined by a specific combination of major and minor alleles in the haplotype block, with the haplotype frequency indicated on the right and the gene loci (±10 kbp) indicated on the top. The triangle indicates the location of rs1921622. (FIG. 10C) Fine-mapping plots showing the associations between variants in the IL1RL1 gene and plasma soluble ST2 (sST2) level. Dot color intensity indicates the effect size of individual variants on plasma sST2 level, and dot size indicates the probability of a variant exerting its causal effect.

FIGS. 11A to 11H. Target deletion at the rs1921622 locus decreases sST2 expression and secretion in brain endothelial cells. (FIG. 11A) Effects of the rs1921622 A allele on the transcript levels of soluble ST2 (sST2) (ENST00000311734.2) in human tissues from the GTEx dataset. The effect size of the rs1921622 A allele and 95% confidence intervals for each tissue (see Table 1 for details) are indicated by boxes and lines, respectively. Red and blue indicate significant (P<0.05) and nonsignificant (P≥0.05) associations between rs1921622 genotype and sST2 transcript level, respectively. (b-d) Single-nucleus RNA sequencing analysis revealed an association between rs1921622 and sST2 transcript level in endothelial cells in the human brain. (FIG. 11B) Uniform manifold approximation and projection (UMAP) plot showing the cell types in the human frontal cortex (n=169,496 cells from 21 participants from the UKBBN cohort). Excit, excitatory neurons; Inhibit, inhibitory neurons; Astro, astrocytes; Mic, microglia; Endo, endothelial cells; Oligo, oligodendrocytes; OPC, oligodendrocyte progenitor cells. (FIG. 11C) Expression profiles of sST2 (upper) and CLDN5 (lower) transcripts in the human frontal cortex. (FIG. 11D) Dot plot showing the expression levels of sST2 (upper) and CLDN5 (lower) stratified according to rs1921622 genotype in endothelial cells from the human frontal cortex. Norm. Exp., normalized expression. (e-h) CRISPR/Cas9 genome editing revealed that the rs1921622 locus is important for sST2 expression. (FIG. 11E) Diagram showing the locations of the 2 sgRNA pairs (i.e.. sgRNA-1 and sgRNA-4 for 67-bp deletion [A67 bp]; sgRNA-2 and sgRNA-3 for 38-bp deletion [A38 bp]) targeting the region harboring the rs1921622 locus (red). (FIG. 11F) ChIP-qPCR analysis of H3K27ac changes at the rs1921622 locus after IL-33 administration for 24 h in hCMEC/D3 cells (n=3 per group; unpaired Student's t-test, T=4.593, *P<0.05). Data in bar charts are mean+SEM. (g, h) Deletion of the rs1921622 locus decreases the transcript level and protein secretion of sST2 in hCMEC/D3 cells. (FIG. 11G) sST2 transcript levels (n=6, 8, and 10 isogenic control clones, clones with 38-bp deletion, and clones with 67-bp deletion, respectively; unpaired Student's t-test, T=−5.444 and −7.612 for Δ38 bp vs. control and Δ67 bp vs. control, respectively, ***P<0.001). (FIG. 11H) Secreted sST2 protein levels in conditioned medium (CM) (n=3, 3, and 4 isogenic control lines, lines with 38-bp deletion, and lines with 67-bp deletion, respectively; unpaired Student's t-test, T=−13.450 and −16.030 for Δ38 bp vs. control and A67 bp vs. control, respectively, ***P<0.001).

FIGS. 12A to 12C. IL-33 induces the expression and secretion of sST2 in brain endothelial cells. (a, b) Administration of IL-33 enhances the expression (n=4 per group) (FIG. 12A) and secretion (n=3 per group) (FIG. 12B) of soluble ST2 (sST2) in hCMEC/D3 cells (unpaired Student's t-test, T=6.484 and 28.89 for sST2 transcript and protein levels, respectively, ***P<0.001). Data in bar charts are mean+SEM. CM, conditioned medium; CtrI, control group. (FIG. 12C) ChIP-qPCR analysis of H3K4me3 changes at the sST2 promoter region after IL-33 administration for 24 h in hCMEC/D3 cells (n=3 per group; unpaired Student's t-test, T=29.69, ***P<0.001).

FIGS. 13A to 13C. Validation of the CRISPR/Cas9-based target deletion at the rs1921622-containing region in hCMEC/D3 cells. (FIG. 13A) Diagram showing the locations of the 2 sgRNA pairs (i.e.. sgRNA-1 and sgRNA-4 for 67-bp deletion [Δ67 bp], and sgRNA-2 and sgRNA-3 for 38-bp deletion [A38 bp]) targeting the rs1921622-containg region (red). (FIG. 13B) Gel images of single clones of isogenic control lines (Ctrl), 38-bp deletion lines (A38 bp) and 67-bp deletion lines (A67 bp). L, DNA ladder. (FIG. 13C) Sanger validation of single clones.

FIGS. 14A to 14G. The rs1921622 A allele exerts protective effects against Alzheimer's disease in APOE-4 carriers. (a, b) Forest plot showing the meta-analysis results of the rs1921622 A allele in overall APOE-ε4 carriers (a) and female APOE-E4 carriers (b) from 6 Alzheimer's disease (AD) datasets (N=5,436 healthy controls, N=5,556 patients with AD). Effect sizes (log odds ratio) obtained from independent datasets and meta-analysis are denoted by rectangles and diamonds, respectively. For the independent datasets, horizontal lines indicate 95% confidence intervals, and rectangle size is proportional to the weight used in the meta-analysis. RE2, Han and Eskin's random-effects model. (c) Survival plot of cumulative dementia-free probability in female APOE-ε4 carriers with AD stratified according to rs1921622 genotype (n=58, 108, and 66 G/G, G/A, and A/A carriers from the LOAD cohort, respectively). HR, hazard ratio. (d) Individual cognitive performance (as determined by Mini-Mental State Examination [MMSE] score) of female APOE-ε4 carriers with AD stratified according to rs1921622 genotype (n=80, 97, and 41 G/G, G/A, and A/A carriers from the Chinese_cohort_2, respectively; linear regression test, T=2.644 and 2.207 for A/A vs. G/G and A/A vs. G/A, respectively, *P<0.05, **P<0.01). Data in box-and-whisker plots include maximum, 75^{th} percentile, median, 25^{th} percentile, and minimum values; plus signs (+) denote corresponding mean values. (e) Effect size of the rs1921622 A allele on brain region volumes in female APOE-ε4 carriers with cognitive impairment (n=29, 72, and 46 G/G, G/A, and A/A carriers from the ADNI cohort, respectively). For each brain region, the effect size of the rs1921622 A allele and 95% confidence intervals are indicated by a box and line, respectively. Red and blue indicate significant (*P<0.05) and nonsignificant (P≥0.05) associations between rs1921622 genotype and brain region volumes, respectively. Fusiform, fusiform gyrus; MidTemp, middle temporal gyrus. (f) Individual cognitive scores in amyloid-beta (Aβ)⁺ APOE-ε4 carriers stratified according to rs1921622 genotype (n=133 G carriers [G/-], n=57 A/A carriers from the AIBL cohort; Wilcoxon rank-sum test, test for the effects of rs1921622 genotype on AIBLPACC score, attention processing score, episodic recall score, and recognition score: W=4336.0, 1970.5, 1610.0, and 1523.5, respectively; *P<0.05, **P<0.01). (g) Longitudinal gray matter volume in Aβ⁺ APOE-ε4 carriers stratified according to rs1921622 genotype (n=281 and 112 datapoints from 128 G carriers [G/-] and 55 A/A carriers from the AIBL cohort, respectively; linear mixed model test, β=−0.046, F=4.839, *P<0.05).

FIG. 15. Alzheimer's disease risk is not associated with rs1921622 genotype in APOE-ε4 noncarriers. Forest plot showing the meta-analysis of rs1921622 in APOE-ε4 noncarriers from the 6 Alzheimer's disease datasets. Values of effect size (log odds ratio) obtained from independent datasets and meta-analysis results are denoted by rectangles and diamonds, respectively. For the independent dataset, the horizontal lines indicate the range of 95% confidence intervals, and rectangle size is proportional to the weight used in the meta-analysis. RE2, Han and Eskin's random effects model.

FIG. 16. The rs1921622 A allele is associated with delayed onset age of dementia in female APOE-ε4 carriers with Alzheimer's disease in the LOAD and ADC cohorts. (a, b) Survival plots of cumulative dementia-free probability in overall APOE-ε4 carriers (n=82, 164, and 99 G/G, G/A, and 99 A/A carriers, respectively) (a) and female APOE-ε4 carriers (n=197, 477, and 276 G/G, G/A, and A/A carriers, respectively) (b) in the Alzheimer's disease (AD) group from the LOAD cohort stratified according to rs1921622 genotype. (c, d) Survival plot of cumulative dementia-free probability in overall APOE-ε4 carriers (n=438, 959, and 543 G/G, G/A, and A/A carriers, respectively) (c) and female APOE-ε4 carriers (n=197, 477, and 276 G/G, G/A, and A/A carriers, respectively) in the AD group from the ADC cohort stratified according to rs1921622 genotype. Cox regression test. HR, hazard ratio.

FIG. 17. The rs1921622 A allele protects against atrophy of the entorhinal cortex in female APOE-ε4 carriers with cognitive impairment in the ADNI cohort. (a) Effects of the rs1921622 A allele on brain region volumes in APOE-ε4 carriers with cognitive impairment (n=374 participants from the ADNI cohort). For each brain region, the effect size of the rs1921622 A allele and 95% confidence intervals are indicated by boxes and lines, respectively. (b) Individual intracranial volume (ICV)-normalized entorhinal cortex volumes in APOE-ε4 carriers with cognitive impairment stratified according to rs1921622 genotype (n=80, 180, and 114 G/G, G/A, and A/A carriers from the ADNI cohort, respectively; linear regression test, T=0.330). Data in box-and-whisker plots include maximum, 75^{th} percentile, median, 25^{th} percentile, and minimum values; plus signs (+) denote corresponding mean values. (c) Effects of the rs1921622 A allele on brain region volumes in female APOE-ε4 carriers with cognitive impairment (n=147 participants from the ADNI cohort). (d) Individual ICV-normalized entorhinal cortex volumes in female APOE-ε4 carriers with cognitive impairment stratified according to rs1921622 genotype (n=29, 72, and 46 G/G, G/A, and A/A carriers from the ADNI cohort, respectively; linear regression test, T=2.285, *P<0.05). Fusiform, fusiform gyrus; MidTemp, middle temporal gyrus.

FIG. 18. The rs1921622 A allele protects against neurodegeneration in female APOE-ε4 carriers with Alzheimer's disease in the Chinese_cohort_1. (a, b) Individual plasma levels of P-tau181 (n=14, 32, and 11 G/G, G/A, and A/A carriers, respectively) (a) and NfL (neurofilament light polypeptide) (n=14, 33, and 12 G/G, G/A, and A/A carriers, respectively) (b) in APOE-ε4 carriers with Alzheimer's disease (AD) from the Chinese_cohort_1 stratified according to rs1921622 genotype (linear regression test, T=−0.419 and −1.622 for plasma P-tau181 and NfL, respectively). Data in box-and-whisker plots include maximum, 75^{th} percentile, median, 25^{th} percentile, and minimum values; plus signs (+) denote corresponding mean values. (c, d) Individual plasma levels of P-tau181 (n=13, 23, and 4 G/G, G/A, and A/A carriers, respectively) (c) and NfL (n=13, 23, and 5 G/G, G/A, and A/A carriers, respectively) in female APOE-ε4 carriers with AD from the Chinese_cohort_1 stratified according to rs1921622 genotype (linear regression test, T=−2.065 and −2.498 for plasma P-tau181 and NfL, respectively; *P<0.05).

FIG. 19. The rs1921622 A allele protects against cognitive decline and gray matter atrophy in female APOE-ε4 carriers in the AIBL Aβ⁺ cohort. (a, b) Individual cognitive scores and baseline gray matter volumes stratified according to rs1921622 genotype among amyloid-beta (Aβ)⁺ APOE-ε4 carriers in the AIBL cohort (n=45, 88, and 57 G/G, G/A, and A/A carriers, respectively; Wilcoxon rank-sum test, tests for rs1921622 (A/A) on AIBLPACC score, Attention Processing score, Episodic Recall score, and Recognition score: W=4,336, 1,970.5, 1,610, and 1,523.5, respectively, *P<0.05, **P<0.01). (c, d) Individual cognitive scores and baseline gray matter volumes stratified according to rs1921622 genotype in female Aβ⁺ APOE-ε4 carriers in the AIBL cohort (n=23, 45, and 33 G/G, G/A, and A/A carriers, respectively; Wilcoxon rank-sum test, tests for rs1921622 (A/A) on AIBLPACC score, Attention Processing score, Episodic Recall score, Recognition score, and baseline gray matter volumes: W=641, 692.5, 584, 572, and 819, respectively; *P<0.05, **P<0.01). Data are presented as box-and-whisker plots including maximum, 75^{th} percentile, median, 25^{th} percentile, and minimum values; plus signs (+) denote corresponding mean values.

FIG. 20. Co-harboring of the rs1921622 A allele restores the impaired microglial activities towards Aβ in female APOE-ε4 carriers with Alzheimer's disease. (a, b) APOE-ε4 is associated with increased amyloid-beta (Aβ) deposition in female patients with Alzheimer's disease (AD). (a) Representative images of Aβ plaque staining in the frontal cortex. Scale bar, 200 μm. (b) Quantification of Aβ plaque load in the frontal cortex stratified according to sex and APOE-ε4 genotype (n=14 male APOE-ε4 noncarriers [non-ε4], n=22 male APOE-ε4 carriers [ε4], n=19 female non-APOE-ε4 carriers, n=23 female APOE-ε4 carriers from the UKBBN cohort; linear regression test, test for the effects of sex:T=2.507 and 5.022 in non-APOE-ε4 and APOE-ε4, respectively, #P<0.05, ###P<0.001; test for the effects of APOE-ε4: T=−0.802 and 2.062 in males and females, respectively, *P<0.05). Data in box-and-whisker plots include maximum, 75^{th} percentile, median, 25^{th} percentile, and minimum values; plus signs (+) denote corresponding mean values. (c) Representative images of Aβ plaque staining in the frontal cortex of female patients with AD stratified according to APOE-ε4 and rs1921622 genotype. Scale bar, 5 mm. (d, e) The rs1921622 variant is associated with increased microglial coverage of Aβin APOE-ε4 carriers but not in noncarriers. (d) Representative image of co-staining of Aβ plaques (brown) and Iba-1⁺ microglia (purple) in the frontal cortex of female APOE-ε4 noncarriers stratified according to rs1921622 genotype. Scale bar, 100 μm. (e) Quantification of the area of Aβplaques colocalized with microglia stratified according to APOE-ε4 genotype and rs1921622 genotype (n=4 and 15 G/G carriers and A carriers [A/-] among APOE-ε4 noncarriers [non-e4], respectively, n=5 and 18 G/G carriers and A/- carriers among APOE-ε4 carriers [e4], respectively, from the UKBBN cohort; linear regression test, test for the effects of APOE-ε4: T=−2.991 and −1.553 in G/G and A/-, respectively, #P<0.05; test for the effects of rs1921622: T=0.229 and 2.409 in non-e4 and e4, respectively, *P<0.05).

FIG. 21. The rs1921622 A allele enhances microglial activities towards Aβ in female APOE-ε4 carriers with Alzheimer's disease. (a, b) The rs1921622 A allele is associated with decreased amyloid-beta (Aβ) deposition in female APOE-ε4 carriers with Alzheimer's disease (AD) (n=4 and 15 G/G carriers and A carriers [A/-] among APOE-ε4 noncarriers [non-ε4], respectively; n=5 and 18 G/G carriers and A/- carriers among APOE-ε4 carriers [ε4], respectively from the UKBBN cohort). (a) Representative images showing Aβ plaque staining in the frontal cortex. Scale bar, 200 μm. (b) Quantification of Aβ plaque area in the frontal cortex stratified according to APOE-ε4 and rs1921622 genotype (linear regression test; effects of APOE-ε4: T=2.912 and 0.956 in G/G and A/- carriers, respectively, #P<0.05; effects of rs1921622: T=−0.833 and −3.706 in non-APOE-ε4 and APOE-ε4 carriers, respectively, *P<0.05). Data in box-and-whisker plots include maximum, 75^{th} percentile, median, 25^{th} percentile, and minimum values; plus signs (+) denote corresponding mean values. (c, d) The rs1921622 A allele is associated with increased microglial colocalization with AD in female APOE-ε4 carriers with AD (n=5 and 18 G/G and A/- carriers among APOE-ε4 carriers from the UKBBN cohort, respectively). (c) Representative image showing co-staining of Aβ plaques (brown) and Iba-1⁺ microglia (purple) in the frontal cortex. Scale bar, 100 μm. (d) Quantification of colocalization between Aβ plaque area and microglia stratified according to rs1921622 genotype (linear regression test, T=2.409, *P<0.05). (e) Volcano plot showing the associations between rs1921622 genotypes and genes expressed by microglia in the frontal cortices of female APOE-ε4 carriers with AD (n=2,636 microglia from 8 participants from the UKBBN cohort). Blue and red dots indicate microglial genes that are negatively and positively associated with the rs1921622 A allele, respectively. Dot size is proportional to the false discovery rate (FDR, log₁₀ scale). The top 5 negatively and positively associated microglial genes are labeled. (f) Representative Gene Ontology (GO) terms enriched in the rs1921622-associated microglial genes. GO terms enriched for the down- and upregulated microglial genes in rs1921622 A allele carriers are indicated in blue and red, respectively. (g) Dot plot showing the expression levels of microglial activation genes and homeostatic genes stratified according to rs1921622 genotype.

FIG. 22. The rs1921622 A allele and cerebrospinal fluid sST2 level exhibit opposite effects on the microglial transcriptome in female APOE-ε4 carriers with Alzheimer's disease. Scatterplot showing the correlation between the normalized effect size (β) of cerebrospinal fluid (CSF) soluble ST2 (sST2) levels and the rs1921622 A allele on microglial gene expression in the frontal cortex of female APOE-ε4 carriers with Alzheimer's disease (AD) (n=2,636 microglia from 8 individuals from the UKBBN cohort).

FIG. 23. Deletion of 3′untranslated region of sST2 reduces sST2 level and alleviates amyloid associated pathologies in 5XFAD mice. (a, b) Deletion of 3′untranslated region of sST2 reduces serum sST2 level. (a) Schematic of deletion of 3′untranslated region of sST2. sST2 and ST2L share most of the coding sequence while the 3′untranslated region of sST2 is specific. (b) Quantitative analysis of serum sST2 level by ELISA. Values are mean±s.e.m (***P<0.001, unpaired two=tailed t-test). (e-h) Deletion of 3′ untranslated region of sST2 alleviates amyloid-associated pathology in 5XFAD mice. (c, d) Quantitative analysis of soluble and insoluble Aβₓ₋₄₀ and Aβₓ₋₄₂ by ELISA. Values are mean±s.e.m (*P<0.05, unpaired two=tailed t-test). (e, f) DAB staining of Aβ. (e) Representative images. (f) Quantification of Aβ plaques (% of total cortical area). Values are mean±s.e.m (*P<0.05, unpaired two=tailed t-test). (g, h) X-34-stained AD deposits. (g) Representative images. (h) Quantification of X-34-positive Aβ plaques (% of total cortical area). Values are mean±s.e.m (*P<0.05, unpaired two=tailed t-test).

FIG. 24. Antisense Oligonucleotides targeting 3′untranslated region of sST2 reduce sST2 level and alleviate amyloid pathology. (a) Schematic of Antisense Oligonucleotides (ASOs) target 3′untranslated region of sST2. (b, c) systematic screening of mouse sST2-ASOs in mouse fibroblast NIH-3T3 cells. (b) sST2 transcript level in all moue sST2 ASOs(c) sST2 transcript level in top 13 efficient ASOs. Values are mean±s.e.m. (d) systemic delivery of sST2-ASO reduces serum sST2 level in vivo. Values are mean±s.e.m. (e, f) X-34-stained Aβ deposits. (e) Representative images. (f) Quantification of X34-positive Aβ plaques (% of total cortical area).

FIG. 25. Antisense Oligonucleotides targeting 3′untranslated region of sST2 reduce sST2 protein and transcript level in human cells. (a) Schematic of Antisense Oligonucleotides (ASOs) target 3′untranslated region of sST2. (b, c) systematic screening of human sST2-ASOs in Human Umbilical Vein Endothelial Cells (HUVEC). (b) Level of secreted sST2 in the medium. The values are normalized the sST2 level in HUVEC transfected with negative control ASO. (c) sST2 transcript level in top 15 efficient ASOs. Values are mean±s.e.m.

Table 1. Associations between the rs1921622 A allele and soluble ST2 and full-length ST2 transcript levels in human tissues. Linear regression test, adjusted for age, sex, RNA integrity, and population structure. β, effect size; SE, standard error; sST2, soluble ST2; ST2L, full-length ST2. Bold and red text indicates statistical significance at a cutoff of P<0.05.^{a} Only tissues in which >50% of individuals exhibited expression (reads per kilobase per million mapped reads [RPKM]>0) were included to calculate the average expression of candidate genes and the association test between candidate gene levels and rs1921622 genotypes.^{b} Average expression of candidate genes among individuals in each tissue.

Table 2. Demographic characteristics of the Chinese_cohort_1. CVD, cardiovascular disease; ICV, intracranial volume; MoCA, Montreal Cognitive Assessment; MRI, magnetic resonance imaging; NfL, neurofilament light polypeptide; SD, standard deviation; sST2, soluble ST2; WGS, whole-genome sequencing.

Table 3. Demographic characteristics of the UK Brain Bank Network cohort. SD, standard deviation; PMD, postmortem duration; CSF, cerebrospinal fluid; sST2, soluble ST2.

Table 4. Candidate genetic variants (P<1E-5) associated with plasma sST2 levels in the Chinese_cohort_1. Linear regression test, adjusted for age, sex, AD diagnosis, and population structure.

Table 5. Candidate genetic variants in the IL1RL1 gene associated with plasma soluble ST2 level after fine mapping (causal probability >0.001). Linear regression test, adjusted for age, sex, AD diagnosis, and population structure, with fine-mapping analysis. β, effect size; SE, standard error; SNP, single nucleotide polymorphism; sST2, soluble ST2. a Probability of being a putative causal variant for plasma sST2 level.

Table 6. Demographic characteristics of the 5 Alzheimer's disease datasets for meta-analysis. MMSE, Mini-Mental State Exam; SD, standard deviation.

Table 7. Minor allele frequency of the rs1921622 A allele in overall participants, APOE-ε4 carriers, and APOE-ε4 noncarriers in the 6 Alzheimer's disease datasets. AD, Alzheimer's disease; HC, healthy control; OR, odds ratio. Bold text indicates statistical significance at a cutoff of P<0.05.

Table 8. Minor allele frequencies of the rs1921622 A allele in male and female APOE-ε4 carriers in the 6 Alzheimer's disease datasets. AD, Alzheimer's disease; HC, healthy control; OR, odds ratio. Bold text indicates statistical significance at a cutoff of P<0.05.

Table 9: oligonucleotide sequences.

### Definitions

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

The term “gene” means the segment of DNA involved in producing a polypeptide chain. It may include regions preceding and following the coding region (leader and trailer) as well as intervening sequences (introns) between individual coding segments (exons).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds having a structure that is different from the general chemical structure of an amino acid, but that functions in a manner similar to a naturally occurring amino acid.

There are various known methods in the art that permit the incorporation of an unnatural amino acid derivative or analog into a polypeptide chain in a site-specific manner, see. e.g., WO 02/086075.

Amino acids may be referred to herein by either the commonly known three letter symbols or by the one-letter symbols recommended by the IUPAC-IUB Biochemical Nomenclature Commission. Nucleotides, likewise, may be referred to by their commonly accepted single-letter codes.

“Polypeptide,” “peptide,” and “protein” are used interchangeably herein to refer to a polymer of amino acid residues. All three terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

As used herein, a “composition disrupting a genomic sequence encompassing rs1921622” refers to any composition comprising one or more agents capable of suppressing or eliminating the transcription or translation of the genomic sequence, which may be achieved by the direct deletion or alternation of at least a portion of the genomic sequence (e.g., by genomic editing technique such as the clustered regularly interspaced short palindromic repeat (CRISPR) system or the like) or may be achieved via reduction or elimination of the mRNA transcribed from the genomic sequence through the action of small inhibitory DNA or RNA molecules or other enzymes (e.g., antisense oligonucleotides, small inhibitory RNAs such as siRNA or shRNA, and ribozymes etc.). Terms and phrases that are used in this disclosure and similarly worded in reference to other genetic loci (such as “other sST2-associated genetic variants listed in Table 4 and Table 5”) are defined in a functionally identical or similar fashion.

The term “targeting,” when used in the context of describing an inhibitory oligonucleotide (such as a small inhibitory RNA or an antisense oligonucleotide) or an sgRNA in relation to a genomic sequence that the inhibitory oligonucleotide or gene editing system is used to negatively regulate, refers to a sufficient sequence complementarity between at least a portion of the oligonucleotide or sgRNA and the genomic sequence, e.g., at least 80, 85, 90, 95% or higher percentage of nucleotide sequence complementarity based on the Watson-Crick base-pairing principle, so as to allow specific hybridization between the sgRNA or oligonucleotide and the genomic sequence or its mRNA transcript, which subsequently leads to the cleavage of the genomic sequence at a pre-determined location or the destruction of its mRNA transcript.

The term “recombinant” when used with reference, e.g., to a cell, or a nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

A “promoter” is defined as an array of nucleic acid control sequences that direct transcription of a polynucleotide sequence. As used herein, a promoter includes necessary polynucleotide sequences near the start site of transcription, such as, in the case of a polymerase II type promoter, a TATA element. A promoter also optionally includes distal enhancer or repressor elements, which can be located as much as several thousand base pairs from the start site of transcription. A “constitutive” promoter is a promoter that is active under most environmental and developmental conditions. An “inducible” promoter is a promoter that is active under environmental or developmental regulation. The term “operably linked” refers to a functional linkage between a polynucleotide expression control sequence (such as a promoter, or array of transcription factor binding sites) and a second polynucleotide sequence, wherein the expression control sequence directs transcription of the polynucleotide sequence corresponding to the second sequence.

An “expression cassette” is a nucleic acid construct, generated recombinantly or synthetically, with a series of specified polynucleotide elements that permit transcription of a particular polynucleotide sequence in a host cell. An expression cassette may be part of a plasmid, viral genome, or nucleic acid fragment. Typically, an expression cassette includes a polynucleotide to be transcribed, operably linked to a promoter.

The term “heterologous” as used in the context of describing the relative location of two elements, refers to the two elements such as polynucleotide sequences (e.g., a promoter and an mRNA- or a protein/polypeptide-encoding sequence) or polypeptide sequences (e.g., two peptides as fusion partners within a fusion protein) that are not naturally found in the same relative positions. Thus, a “heterologous promoter” of a coding sequence refers to a promoter that is not naturally operably linked to that coding sequence. Similarly, a “heterologous polypeptide” or “heterologous polynucleotide” to a particular protein or its coding sequence is one derived from an origin that is different from that particular protein, or if derived from the same origin but not naturally connected to that particular protein or its coding sequence in the same fashion. The fusion of one polypeptide (or its coding sequence) with a heterologous polypeptide (or polynucleotide sequence) does not result in a longer polypeptide or polynucleotide sequence that can be found in nature.

The phrase “specifically hybridize(s) to” refers to the binding, duplexing, or hybridization of one polynucleotide sequence to another polynucleotide sequence based on Watson-Crick nucleotide base-pairing under stringent hybridization conditions when that sequences are present in a complex mixture (e.g., total cellular or library DNA or RNA). The phrase “stringent hybridization conditions” refers to conditions under which a nucleic acid (e.g., a polynucleotide probe) will hybridize to its target nucleotide sequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For high stringency hybridization, a positive signal is at least two times background, preferably 10 times background hybridization. Exemplary high stringency hybridization conditions include: 50% formamide, 5×SSC and 1% SDS incubated at 42° C. or 5×SSC and 1% SDS incubated at 65° C., with a wash in 0.2×SSC and 0.1% SDS at 65° C.

By “host cell” is meant a cell that contains an expression vector and supports the replication or expression of the expression vector. Host cells may be prokaryotic cells such as E. coli, or eukaryotic cells such as yeast, insect, amphibian, or mammalian cells such as CHO, HeLa and the like, e.g., cultured cells, explants, and cells in vivo.

The term “inhibiting” or “inhibition,” as used herein, refers to any detectable negative effect an inhibitory agent has on a target biological process, such as expression of soluble ST2 (sST2) protein, formation of amyloid β (Aβ) plaques in an AD patient's brain, an AD patient's cognitive decline, protein phosphorylation, cellular signal transduction, protein synthesis, cell proliferation, tumorigenicity, and metastatic potential etc. Typically, an inhibition is reflected in a decrease of at least 10%, 20%, 30%, 40%, or 50% in target process (e.g., sST2 protein expression or Aβ plaque accumulation), or any one of the downstream parameters mentioned above, when compared to a control not exposed to the inhibitory agent. In a similar fashion, the term “increasing” or “increase” is used to describe any detectable positive effect an enhancing agent has on a target biological process, such as a positive change of at least 25%, 50%, 75%, 100%, or as high as 2, 3, 4, 5 or up to 10 or 20 fold, when compared to a control in the absence of the enhancer. Conversely, the term “substantially unchanged” describes a state in which the positive or negative changes are less than 10%, 5%, 2%, 1% or lower.

The term “effective amount,” as used herein, refers to an amount that is sufficient to produces an intended effect for which a substance is administered. The effect may include a desirable change in a biological process (e.g., a detectable decrease of sST2 expression, reduction in Aβ plaque formation, or slowing of cognitive decline in an AD patient) as well as the prevention, correction, or inhibition of progression of the symptoms of a disease/condition and related complications to any detectable extent. The exact amount “effective” for achieving a desired effect will depend on the nature of the therapeutic agent, the manner of administration, and the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); and Pickar, Dosage Calculations (1999)).

As used herein, the term “treatment” or “treating” includes both therapeutic and preventative measures taken to address the presence of a disease or condition or the risk of developing such disease or condition at a later time. It encompasses therapeutic or preventive measures for alleviating ongoing symptoms, inhibiting or slowing disease progression, delaying of onset of symptoms, or eliminating or reducing side-effects caused by such disease or condition. A preventive measure in this context and its variations do not require 100% elimination of the occurrence of an event; rather, they refer to a suppression or reduction in the likelihood or severity of such occurrence or a delay in such occurrence.

A “pharmaceutically acceptable” or “pharmacologically acceptable” excipient is a substance that is not biologically harmful or otherwise undesirable, i.e., the excipient may be administered to an individual along with a bioactive agent without causing any undesirable biological effects. Neither would the excipient interact in a deleterious manner with any of the components of the composition in which it is contained.

The term “excipient” refers to any essentially accessory substance that may be present in the finished dosage form of the composition of this invention. For example, the term “excipient” includes vehicles, binders, disintegrants, fillers (diluents), lubricants, glidants (flow enhancers), compression aids, colors, sweeteners, preservatives, suspending/dispersing agents, film formers/coatings, flavors and printing inks.

The term “consisting essentially of,” when used in the context of describing a composition containing an active ingredient or multiple active ingredients, refer to the fact that the composition does not contain other ingredients possessing any similar or relevant biological activity of the active ingredient(s) or capable of enhancing or suppressing the activity, whereas one or more inactive ingredients such as physiological or pharmaceutically acceptable excipients may be present in the composition. For example, a composition consisting essentially of active agent(s) effective for disrupting an rs1921622-containing genomic sequence or for suppressing mRNA transcribed from the genomic sequence in a subject is a composition that does not contain any other agents that may have any detectable positive or negative effect on the same target process or that may increase or decrease to any measurable extent of the disease occurrence or symptoms among the receiving subjects.

The term “about” denotes a range of +/−10% of a pre-determined value. For example, “about 10” sets a range of 90% to 110% of 10, i.e., 9 to 11.

### DETAILED DESCRIPTION OF THE INVENTION

### I. Introduction

In their earlier studies the present inventors discovered the correlation between increased soluble ST2 (sST2) protein serum level and increased amyloid β (Aβ) plaque formation in the brain of Alzheimer's Disease (AD) patients as well as the anti-AD protective effects of A allele at rs1921622 as manifested in a marked reduction of serum sST2 level and Aβ plaque accumulation, especially within certain segments of the general population (for example, among female APOE-ε4 carriers), see, e.g., WO2017/009750 and WO2021/037027. The inventors have now further discovered that the disruption of a genomic sequence encompassing rs1921622 and/or other sST2-associated genetic variants listed in Table 4 and Table 5 can serve as an effective means of directly suppressing sST2 protein expression and secretion in the brain endothelial cells. It is therefore demonstrated that such disruption of rs1921622 genetic locus provides therapeutic benefits in the treatment of patients suffering from Alzheimer's Disease as well as prophylactic benefits in the prevention or risk reduction of Alzheimer's Disease in individuals who are not yet diagnosed of the disease.

### II. General Recombinant Technology

Basic texts disclosing general methods and techniques in the field of recombinant genetics include Sambrook and Russell, Molecular Cloning. A Laboratory Manual (3rd ed. 2001); Kriegler, Gene Transfer and Expression: A Laboratory Manual (1990); and Ausubel et al., eds., Current Protocols in Molecular Biology (1994).

For nucleic acids, sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Proteins sizes are estimated from gel electrophoresis, from sequenced proteins, from derived amino acid sequences, or from published protein sequences.

Oligonucleotides that are not commercially available can be chemically synthesized, e.g., according to the solid phase phosphoramidite triester method first described by Beaucage & Caruthers, Tetrahedron Lett. 22: 1859-1862 (1981), using an automated synthesizer, as described in Van Devanter et. al., Nucleic Acids Res. 12: 6159-6168 (1984). Purification of oligonucleotides is performed using any art-recognized strategy, e.g., native acrylamide gel electrophoresis or anion-exchange HPLC as described in Pearson & Reanier, J. Chrom. 255: 137-149 (1983).

The sequence of a gene of interest, a polynucleotide encoding a polypeptide of interest, and synthetic oligonucleotides can be verified after cloning or subcloning using, e.g., the chain termination method for sequencing double-stranded templates of Wallace et al., Gene 16: 21-26 (1981).

### II. Composition Disrupting a Genomic Sequence

Earlier work by the present inventors illustrated the involvement of sST2 protein and genetic locus rs1921622 in the development of Alzheimer's Disease. Their latest discovery reveals that, by disrupting the genomic sequence encompassing the genetic locus rs1921622 as well as other genomic sites listed in Table 4 and Table 5, sST2 expression and Aβ plaque accumulation in the brain can be reduced. This revelation leads to the therapeutic and prophylactic use of compositions disrupting this genomic sequence, which may act at the level of intact genomic sequence at or immediately surrounding the genetic locus of rs1921622, and/or other candidate genomic sites listed in Table 4 and Table 5, or which may act at the level of mRNA transcribed from the genomic sequence, for treating Alzheimer's Disease in patients already diagnosed with the disease and preventing/reducing risk of later developing Alzheimer's Disease in individuals who have not yet received a diagnosis but are at heightened risk for the disease, e.g., due to family history or known genetic background (for instance, carrier of one or two APOE ε4 alleles, point mutation in the genomic sequence encoding amyloid precursor protein (APP) on chromosome 21, point mutation in the genomic sequence encoding Presenilin 1 (PSEN1) on chromosome 14, and point mutation in the genomic sequence encoding Presenilin 2 (PSEN2) on chromosome 1). Various categories of possible agents acting through different mechanisms (e.g., by genomic editing or mRNA suppression) are useful in formulating such compositions for the disruption of the rs1921622-containing genomic sequence and are discussed below.

### A. Antisense Oligonucleotides

In some embodiments, the agent is an antisense oligonucleotide. Antisense oligonucleotides are relatively short nucleic acids that are complementary (or antisense) to the coding strand (sense strand) of the RNA transcribed from the genomic sequence encompassing sST2 gene, e.g., rs1921622 or the 3′ untranslated regions (3′-UTR) of sST2 gene (Chr2: 102,959,893-102,961,182). Although antisense oligonucleotides are typically RNA based, they can also be DNA based. Also, antisense oligonucleotides are often modified to increase their stability.

Without being bound by theory, the binding of these relatively short oligonucleotides to the mRNA is believed to induce stretches of double stranded RNA that trigger degradation of the messages by endogenous RNAses. Additionally, sometimes the oligonucleotides are specifically designed to bind near the promoter of the coding sequence, and under these circumstances, the antisense oligonucleotides may additionally interfere with translation of the mRNA. Regardless of the specific mechanism by which antisense oligonucleotides function, their administration to a cell or tissue allows the degradation of the RNA transcribed from the genomic sequence encompassing sST2 gene, for example, rs1921622 or the 3′ UTR of sST2 gene (Chr2: 102,959,893-102,961,182). Accordingly, antisense oligonucleotides decrease the expression and/or activity of encoded product from the genomic sequence.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; WO 88/09810) or the blood-brain barrier (see, e.g., WO 89/10134), hybridization-triggered cleavage agents (See, e.g., Krol et al., 1988, BioTechniques 6:958-976) or intercalating agents (see, e.g., Zon, 1988, Pharm. Res. 5:539-549). To this end, the oligonucleotide can be conjugated to another molecule.

Oligonucleotides of the invention may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16:3209), methylphosphonate oligonucleotides can be prepared by use of controlled pore glass polymer supports (Sarin et al., 1988, Proc. Natl. Acad. Sci. U.S.A. 85:7448-7451) etc.

A number of methods have been developed for delivering antisense DNA or RNA to cells; e.g., antisense molecules can be injected directly into the target anatomic site, or modified antisense molecules, designed to target the desired cells (e.g., antisense linked to peptides or antibodies that specifically bind receptors or antigens expressed on the target cell surface) can be administered systematically.

It may be difficult to achieve intracellular concentrations of the antisense sufficient to suppress translation on endogenous mRNAs in certain instances. Therefore, another approach utilizes a recombinant DNA construct in which the antisense oligonucleotide is placed under the control of a strong pol III or pol II promoter. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human cells. Such promoters can be inducible or constitutive. Such promoters include but are not limited to: the SV40 early promoter region (Bemoist and Chambon, 1981, Nature 290:304-310), the promoter contained in the 3′ long terminal repeat of Rous sarcoma virus (Yamamoto et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al., 1982, Nature 296:39-42), etc. Any type of plasmid, cosmid, YAC or viral vector can be used to prepare the recombinant DNA construct that can be introduced directly into the target tissue site. Alternatively, viral vectors can be used which selectively infect the desired tissue, in which case administration may be accomplished by another route (e.g., systematically).

### B. Small Interfering RNA

In some embodiments, the agent is a small interfering RNA (siRNA or RNAi) molecule. RNAi constructs comprise double stranded RNA that can specifically block expression of a target gene. “RNA interference” or “RNAi” is a term initially applied to a phenomenon where double-stranded RNA (dsRNA) blocks gene expression in a specific and post-transcriptional manner. RNAi provides a useful method of inhibiting gene expression in vitro or in vivo. RNAi constructs can include small interfering RNAs (siRNAs), short hairpin RNAs (shRNAs), and other RNA species that can be cleaved in vivo to form siRNAs. RNAi constructs herein also include expression vectors (“RNAi expression vectors”) capable of giving rise to transcripts which form dsRNAs or hairpin RNAs in cells, and/or transcripts which can produce siRNAs in vivo.

RNAi expression vectors express (transcribe) RNA which produces siRNA moieties in the cell in which the construct is expressed. Such vectors include a transcriptional unit comprising an assembly of (1) genetic element(s) having a regulatory role in gene expression, for example, promoters, operators, or enhancers, operatively linked to (2) a “coding” sequence which is transcribed to produce a double-stranded RNA (two RNA moieties that anneal in the cell to form an siRNA, or a single hairpin RNA, which can be processed to an siRNA), and (3) appropriate transcription initiation and termination sequences. The choice of promoter and other regulatory elements generally varies according to the intended host cell.

The RNAi constructs contain a nucleotide sequence that hybridizes under physiologic conditions of the cell to the nucleotide sequence of at least a portion of the mRNA transcript for the gene to be inhibited (i.e., an RNA transcribed from a genomic sequence encompassing rs1921622 or the 3′ UTR of the sST2 gene). The double-stranded RNA need only be sufficiently similar to natural RNA that it has the ability to mediate RNAi. Thus, the invention has the advantage of being able to tolerate sequence variations that might be expected due to genetic mutation, strain polymorphism or evolutionary divergence. The number of tolerated nucleotide mismatches between the target sequence and the RNAi construct sequence is no more than 1 in 5 basepairs, or 1 in 10 basepairs, or 1 in 20 basepairs, or 1 in 50 basepairs. Mismatches in the center of the siRNA duplex are most critical and may essentially abolish cleavage of the target RNA. In contrast, nucleotides at the 3′ end of the siRNA strand that is complementary to the target RNA do not significantly contribute to specificity of the target recognition.

Production of RNAi constructs can be carried out by chemical synthetic methods or by recombinant nucleic acid techniques. Endogenous RNA polymerase of the treated cell may mediate transcription in vivo, or cloned RNA polymerase can be used for transcription in vitro. The RNAi constructs may include modifications to either the phosphate-sugar backbone or the nucleoside, e.g., to reduce susceptibility to cellular nucleases, improve bioavailability, improve formulation characteristics, and/or change other pharmacokinetic properties. For example, the phosphodiester linkages of natural RNA may be modified to include at least one of an nitrogen or sulfur heteroatom. Modifications in RNA structure may be tailored to allow specific genetic inhibition while avoiding a general response to dsRNA. Likewise, bases may be modified to block the activity of adenosine deaminase. The RNAi construct may be produced enzymatically or by partial/total organic synthesis, any modified ribonucleotide can be introduced by in vitro enzymatic or organic synthesis.

In certain embodiments, the subject RNAi constructs are “small interfering RNAs” or “siRNAs.” These nucleic acids are around 19-30 nucleotides in length, and even more preferably 21-23 nucleotides in length, e.g., corresponding in length to the fragments generated by nuclease “dicing” of longer double-stranded RNAs. The siRNAs are understood to recruit nuclease complexes and guide the complexes to the target mRNA by pairing to the specific sequences. As a result, the target mRNA is degraded by the nucleases in the protein complex. In a particular embodiment, the 21-23 nucleotides siRNA molecules comprise a 3′ hydroxyl group.

In certain embodiments, the RNAi construct is in the form of a short hairpin structure (named as shRNA). The shRNAs can be synthesized exogenously or can be formed by transcribing from RNA polymerase III promoters in vivo. Examples of making and using such hairpin RNAs for gene silencing in mammalian cells are described in, for example, Paddison et al., Genes Dev, 2002, 16:948-58; McCaffrey et al., Nature, 2002, 418:38-9; Yu et al., Proc Natl Acad Sci USA, 2002, 99:6047-52). Often, such shRNAs are engineered in cells or in an animal to ensure continuous and stable suppression of a desired gene. It is known in the art that siRNAs can be produced by processing a hairpin RNA in the cell.

A plasmid can be used to deliver the double-stranded RNA, e.g., as a transcriptional product. In such embodiments, the plasmid is designed to include a “coding sequence” for each of the sense and antisense strands of the RNAi construct. The coding sequences can be the same sequence, e.g., flanked by inverted promoters, or can be two separate sequences each under transcriptional control of separate promoters. After the coding sequence is transcribed, the complementary RNA transcripts base-pair to form the double-stranded RNA.

### C. Ribozymes

In some embodiments, the agent is a ribozyme. Ribozymes molecules designed to catalytically cleave an mRNA transcript are also used to disrupt and prevent the downstream effects of the mRNA (See, e.g., WO 90/11364; Sarver et al., 1990, Science 247:1222-1225 and U.S. Pat. No. 5,093,246). While ribozymes that cleave mRNA at site-specific recognition sequences can be used to destroy particular mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach, 1988, Nature, 334:585-591.

The ribozymes for use in this invention may also include RNA endoribonucleases (hereinafter “Cech-type ribozymes”) such as the one that occurs naturally in Tetrahymena thermophila (known as the IVS, or L-19 IVS RNA) and has been extensively described by Thomas Cech and collaborators (Zaug, et al., 1984, Science, 224:574-578; Zaug and Cech, 1986, Science, 231:470-475; Zaug, et al., 1986, Nature, 324:429-433; WO 88/04300; Been and Cech, 1986, Cell, 47:207-216). The Cech-type ribozymes have an 8-basepair active site that hybridizes to a target RNA sequence whereafter cleavage of the target RNA takes place. The invention encompasses those Cech-type ribozymes that target 8-basepair active site sequences.

As in the antisense approach, the ribozymes can be composed of modified oligonucleotides (e.g., for improved stability, targeting, etc.) and can be delivered to cells in vitro or in vivo. A preferred method of delivery involves using a DNA construct “encoding” the ribozyme under the control of a strong constitutive pol III or pol II promoter, so that transfected cells will produce sufficient quantities of the ribozyme to destroy targeted mRNA and inhibit its effect. Because ribozymes unlike antisense molecules, are catalytic, a lower intracellular concentration is required for efficiency.

There are currently two basic types of DNA enzymes, and both of these were identified by Santoro and Joyce (see, e.g., U.S. Pat. No. 6,110,462). The 10-23 DNA enzyme comprises a loop structure which connect two arms. The two arms provide specificity by recognizing the particular target nucleic acid sequence while the loop structure provides catalytic function under physiological conditions.

Briefly, to design an ideal DNA enzyme that specifically recognizes and cleaves a target nucleic acid, one of skill in the art must first identify the unique target sequence. This can be done using the same approach as outlined for antisense oligonucleotides. Preferably, the unique or substantially sequence is a G/C rich of approximately 18 to 22 nucleotides. High G/C content helps insure a stronger interaction between the DNA enzyme and the target sequence.

When synthesizing the DNA enzyme, the specific antisense recognition sequence that will target the enzyme to the message is divided so that it comprises the two arms of the DNA enzyme, and the DNA enzyme loop is placed between the two specific arms.

Methods of making and administering DNA enzymes can be found, for example, in U.S. Pat. No. 6,110,462. Similarly, methods of delivery DNA ribozymes in vitro or in vivo include methods of delivery RNA ribozyme, as outlined in detail above. Additionally, one of skill in the art will recognize that, like antisense oligonucleotide, DNA enzymes can be optionally modified to improve stability and improve resistance to degradation.

### D. Genomic Editing

The inhibition of sST2 protein expression can be achieved by way of disruption of the genetic sequence encompassing the genetic locus rs1921622, and/or other genomic sites listed in Table 4 and Table 5, or the 3′-UTR of sST2 gene/transcript. One effective means of targeted gene cleavage is the CRISPR system.

The term CRISPR, abbreviation for Clustered Regularly Interspaced Short Palindromic Repeats, was originally coined in reference to segments of prokaryotic DNA that contain short, repetitive base sequences, initially found in bacteria and archaea. In a palindromic repeat, the sequence of nucleotides is the same in both directions. Each repetition is followed by short segments of spacer DNA from previous exposures to foreign DNA (e.g., DNA of a virus). Small clusters of Cas (CRISPR-associated) genes are located next to CRISPR sequences. It was later recognized that the CRISPR/Cas system is a prokaryotic immune system that confers resistance to foreign genetic elements especially those of viral origin and thereby provides a form of acquired immunity. RNA harboring the spacer sequence helps Cas (CRISPR-associated) proteins recognize and cut exogenous DNA. Other RNA-guided Cas proteins cut foreign RNA. CRISPRs are found in approximately 50% of sequenced bacterial genomes and nearly 90% of sequenced archaea, and recently the CRISPR/Cas system have been adapted for use in targeted gene editing in eukaryotic cells. See, e.g., Ledford (2016), Nature 531 (7593):156-9.

A simple version of the CRISPR/Cas system, CRISPR/Cas9, has been modified to edit genomes. By delivering the Cas9 nuclease complexed with one or more synthetic guide RNA (gRNA) into a cell, typically by transfecting the cell with one or more expression vectors encoding for the Cas9 nuclease and the gRNA(s), the cell's genome can be cut at one or more pre-selected location, allowing a target gene (e.g., the genomic sequence harboring rs1921622) to be removed and/or substituted by a new sequence.

In the instant case, an expression vector (for example, a viral vector) carrying the coding sequence for one or more gRNA specific for rs1921622-encompassing genomic sequence and/or other genomic sequence containing the sites listed in Table 4 and Table 5 can be introduced into a cell in which the endogenous rs1921622-containing genomic sequence and/or other genomic sequence containing the sites listed in Table 4 and Table 5 is to be knocked out (for example, an endothelial cell or an endothelial progenitor cell). The same expression vector optionally also carries the coding sequence for the CRISPR/Cas9 nuclease or equivalent. In the alternative, a separate expression vector may be used to introduce the CRISPR/Cas9 nuclease coding sequence for its expression in the target cells. In some cases, more than one (e.g., two) distinct gRNAs are used to ensure removal and/or replacement of a target genomic sequence (e.g., one that encompasses the rs1921622 locus and/or other genomic sites listed in Table 4 and Table 5).

Additional gene editing systems that can be used for practicing the present invention include TALENs (Transcription activator-like effector nucleases), ZFNs (Zinc-finger nucleases), and base editing, as well as newly developed techniques such as homing endonucleases and meganucleases (MegNs) (which target and cleave DNA sequences) and prime editing (which generates RNA templates for gene alteration).

### III. Pharmaceutical Compositions and Administration

The present invention also provides pharmaceutical compositions or physiological compositions comprising an effective amount of one or more agents useful in the methods of the present invention in both prophylactic and therapeutic applications. Such pharmaceutical or physiological compositions also include one or more pharmaceutically or physiologically acceptable excipients or carriers. For instance, one exemplary composition of this invention comprises or consists essentially of one or more expression vectors encoding a CRISPR system (e.g., a Cas9 nuclease or equivalent and one or two sgRNAs) plus one or more physiologically acceptable excipients or carriers. In another exemplary composition of this invention comprises or consists essentially of one or more expression vectors encoding one or more inhibitory oligonucleotides (e.g., a small inhibitory RNA molecule or an antisense DNA or RNA oligonucleotide) plus one or more physiologically acceptable excipients or carriers. Pharmaceutical compositions of the invention are suitable for use in a variety of drug delivery systems. Suitable formulations for use in the present invention are found in Remington's Pharmaceutical Sciences, Mack Publishing Company, Philadelphia, PA, 17th ed. (1985). For a brief review of methods for drug delivery, see, Langer, Science 249: 1527-1533 (1990).

The pharmaceutical compositions of the present invention can be administered by various routes, e.g., oral, subcutaneous, transdermal, intramuscular, intravenous, or intracranial. The preferred routes of administering the pharmaceutical compositions are local delivery to a relevant organ or tissue to the target disease in a recipient at a pre-determined daily dose. The appropriate dose may be administered in a single daily dose or as divided doses presented at appropriate intervals, for example as two, three, four, or more subdoses per day.

For preparing pharmaceutical compositions containing one or more active agents of this invention, inert and pharmaceutically acceptable carriers are also used. Typically, the pharmaceutical carrier can be either solid or liquid. Solid form preparations include, for example, powders, tablets, dispersible granules, capsules, cachets, and suppositories. A solid carrier can be one or more substances that can also act as diluents, flavoring agents, solubilizers, lubricants, suspending agents, binders, or tablet disintegrating agents; it can also be an encapsulating material.

In powders, the carrier is generally a finely divided solid that is in a mixture with the finely divided active component. In tablets, the active ingredient is mixed with the carrier having the necessary binding properties in suitable proportions and compacted in the shape and size desired.

For preparing pharmaceutical compositions in the form of suppositories, a low-melting wax such as a mixture of fatty acid glycerides and cocoa butter is first melted and the active ingredient is dispersed therein by, for example, stirring. The molten homogeneous mixture is then poured into convenient-sized molds and allowed to cool and solidify.

Powders and tablets preferably contain between about 5% to about 70% by weight of the active ingredient. Suitable carriers include, for example, magnesium carbonate, magnesium stearate, talc, lactose, sugar, pectin, dextrin, starch, tragacanth, methyl cellulose, sodium carboxymethyl cellulose, a low-melting wax, cocoa butter, and the like.

The pharmaceutical compositions can include the formulation of the active agent(s) with encapsulating material as a carrier providing a capsule in which the agent or agents (with or without other carriers) is/are surrounded by the carrier, such that the carrier is thus in association with the agent(s). In a similar manner, cachets can also be included. Tablets, powders, cachets, and capsules can be used as solid dosage forms suitable for oral administration.

Liquid pharmaceutical compositions include, for example, solutions suitable for oral or parenteral administration, suspensions, and emulsions suitable for oral administration. Sterile water solutions of the active component (s) or sterile solutions of the active component(s) in solvents comprising water, buffered water, saline, PBS, ethanol, or propylene glycol are examples of liquid compositions suitable for parenteral administration. The compositions may contain pharmaceutically acceptable auxiliary substances as required to approximate physiological conditions, such as pH adjusting and buffering agents, tonicity adjusting agents, wetting agents, detergents, and the like.

Sterile solutions can be prepared by dissolving the active component(s) in the desired solvent system, and then passing the resulting solution through a membrane filter to sterilize it or, alternatively, by dissolving the sterile component(s) in a previously sterilized solvent under sterile conditions. The resulting aqueous solutions may be packaged for use as is, or lyophilized, the lyophilized preparation being combined with a sterile aqueous carrier prior to administration. The pH of the preparations typically will be between 3 and 11, more preferably from 5 to 9, and most preferably from 7 to 8.

The pharmaceutical compositions containing one or more active agents can be administered for prophylactic and/or therapeutic treatments. In therapeutic applications, compositions are administered to a patient already suffering from Alzheimer's Disease in an amount sufficient to prevent, cure, reverse, or at least partially slow or arrest the symptoms of the disease and its complications, such as the onset, progression, duration, and severity of the disease. An amount adequate to accomplish this is defined as a “therapeutically effective dose.” Amounts effective for this use will depend on the severity of the disease, the weight and general state of the patient, as well as the nature of the active agent(s).

In prophylactic applications, pharmaceutical compositions containing one or more active agents are administered to a patient susceptible to or otherwise at risk of developing Alzheimer's Disease in an amount sufficient to delay or prevent the onset of the symptoms. Such an amount is defined to be a “prophylactically effective dose.” In this use, the precise amounts of the active agent(s) again depend on the patient's state of health and weight, as well as the nature of the active agent(s).

Single or multiple administrations of the compositions can be carried out with dose levels and pattern being selected by the treating physician. In any event, the pharmaceutical formulations should provide a quantity of agent(s) sufficient to effectively suppress serum level of sST2 protein and Aβ plaque formation in the patient, either therapeutically or prophylactically.

### IV. Therapeutic Applications Using Nucleic Acids

A variety of conditions can be treated by therapeutic approaches that involve introducing a nucleic acid encoding one or more agents disrupting rs1921622-encompassing genomic sequence, and/or other genomic sequence containing the sites listed in Table 4 and Table 5, or inhibiting mRNA encoded by the genomic sequence (such as antisense or miRNA or Cas9 nuclease and sgRNAs) into a cell such that the coding sequence is transcribed and the polypeptide or oligonucleotide agent is produced in the cell. For discussions on the application of gene therapy towards the treatment of genetic as well as acquired diseases, see, Miller Nature 357:455-460 (1992); and Mulligan Science 260:926-932 (1993).

### A. Vectors for Gene Delivery

For delivery to a cell or organism, a polynucleotide encoding one or more active agents can be incorporated into a vector. Examples of vectors used for such purposes include expression plasmids capable of directing the expression of the nucleic acids in the target cell. In other instances, the vector is a viral vector system wherein the polynucleotide is incorporated into a viral genome that is capable of transfecting the target cell. In one embodiment, the encoding polynucleotide can be operably linked to expression and control sequences that can direct expression of the polypeptide or oligonucleotide in the desired target host cells. Thus, one can achieve expression of the polypeptide or oligonucleotide inhibitor under appropriate conditions in the target cell.

### B. Gene Delivery Systems

Viral vector systems useful in the expression of a polypeptide or oligonucleotide disrupting a genomic sequence encompassing rs1921622, and/or other genomic sites listed in Table 4 and Table 5, include, for example, naturally occurring or recombinant viral vector systems. Depending upon the particular application, suitable viral vectors include replication competent, replication deficient, and conditionally replicating viral vectors. For example, viral vectors can be derived from the genome of human or bovine adenoviruses, vaccinia virus, herpes virus, adeno-associated virus, minute virus of mice (MVM), HIV, sindbis virus, and retroviruses (including but not limited to Rous sarcoma virus and lentivirus), and MoMLV. Typically, the coding sequence of interest (e.g., one encoding for a polypeptide or oligonucleotide active agent of the present invention) are inserted into such vectors to allow packaging of the gene construct, typically with accompanying viral DNA, followed by infection of a sensitive host cell and expression of the coding sequence of interest.

As used herein, “gene delivery system” refers to any means for the delivery of a polynucleotide sequence of the interest to a target cell. In some embodiments of the invention, nucleic acids are conjugated to a cell receptor ligand for facilitated uptake (e.g., invagination of coated pits and internalization of the endosome) through an appropriate linking moiety, such as a DNA linking moiety (Wu et al., J. Biol. Chem. 263:14621-14624 (1988); WO 92/06180), or by ultrasound-microbubble delivery system (Lan H Y et al., J. Am Soc. Nephrol. 14:1535-1548). For example, nucleic acids can be linked through a polylysine moiety to asialo-oromucocid, which is a ligand for the asialoglycoprotein receptor of hepatocytes.

Similarly, viral envelopes used for packaging gene constructs that include the nucleic acids of the interest can be modified by the addition of receptor ligands or antibodies specific for a receptor to permit receptor-mediated endocytosis into specific cells (see. e.g., WO 93/20221, WO 93/14188, and WO 94/06923). In some embodiments of the invention, the DNA constructs of the invention are linked to viral proteins, such as adenovirus particles, to facilitate endocytosis (Curiel et al., Proc. Natl. Acad. Sci. U.S.A. 88:8850-8854 (1991)). In other embodiments, the active agents of the instant invention can include microtubule inhibitors (WO/9406922), synthetic peptides mimicking influenza virus hemagglutinin (Plank et al., J. Biol. Chem. 269:12918-12924 (1994)), and nuclear localization signals such as SV40 T antigen (WO93/19768).

Retroviral vectors may also be useful for introducing the coding sequence of a polypeptide or oligonucleotide active agent of the invention into target cells or tissues. Retroviral vectors are produced by genetically manipulating retroviruses. The viral genome of retroviruses is RNA. Upon infection, this genomic RNA is reverse transcribed into a DNA copy which is integrated into the chromosomal DNA of transduced cells with a high degree of stability and efficiency. The integrated DNA copy is referred to as a provirus and is inherited by daughter cells as is any other gene. The wild-type retroviral genome and the proviral DNA have three genes: the gag, the pol and the env genes, which are flanked by two long terminal repeat (LTR) sequences. The gag gene encodes the internal structural (nucleocapsid) proteins; the pol gene encodes the RNA directed DNA polymerase (reverse transcriptase); and the env gene encodes viral envelope glycoproteins. The 5′ and 3′ LTRs serve to promote transcription and polyadenylation of virion RNAs. Adjacent to the 5′ LTR are sequences necessary for reverse transcription of the genome (the tRNA primer binding site) and for efficient encapsulation of viral RNA into particles (the Psi site) (see, Mulligan, In: Experimental Manipulation of Gene Expression, Inouye (ed), 155-173 (1983); Mann et al., Cell 33:153-159 (1983); Cone and Mulligan, Proceedings of the National Academy of Sciences. U.S.A., 81:6349-6353 (1984)).

The design of retroviral vectors is well known to those of ordinary skill in the art. In brief, if the sequences necessary for encapsidation (or packaging of retroviral RNA into infectious virions) are missing from the viral genome, the result is a cis acting defect which prevents encapsidation of genomic RNA. However, the resulting mutant is still capable of directing the synthesis of all virion proteins. Retroviral genomes from which these sequences have been deleted, as well as cell lines containing the mutant genome stably integrated into the chromosome are well known in the art and are used to construct retroviral vectors. Preparation of retroviral vectors and their uses are described in many publications including, e.g., European Patent Application EPA 0 178 220; U.S. Pat. No. 4,405,712; Gilboa Biotechniques 4:504-512 (1986); Mann et al., Cell 33:153-159 (1983); Cone and Mulligan Proc. Natl. Acad. Sci. USA 81:6349-6353 (1984); Eglitis et al. Biotechniques 6:608-614 (1988); Miller et al. Biotechniques 7:981-990 (1989); Miller (1992) supra; Mulligan (1993), supra; and WO 92/07943.

The retroviral vector particles are prepared by recombinantly inserting the desired nucleotide sequence into a retrovirus vector and packaging the vector with retroviral capsid proteins by use of a packaging cell line. The resultant retroviral vector particle is incapable of replication in the host cell but is capable of integrating into the host cell genome as a proviral sequence containing the desired nucleotide sequence. As a result, the patient is capable of producing, for example, a polypeptide or polynucleotide active agent useful in the methods of the invention and thus restore the target cells (e.g., brain endothelial cells) to a normal phenotype.

Packaging cell lines that are used to prepare the retroviral vector particles are typically recombinant mammalian tissue culture cell lines that produce the necessary viral structural proteins required for packaging, but which are incapable of producing infectious virions. The defective retroviral vectors that are used, on the other hand, lack these structural genes but encode the remaining proteins necessary for packaging. To prepare a packaging cell line, one can construct an infectious clone of a desired retrovirus in which the packaging site has been deleted. Cells comprising this construct will express all structural viral proteins, but the introduced DNA will be incapable of being packaged. Alternatively, packaging cell lines can be produced by transforming a cell line with one or more expression plasmids encoding the appropriate core and envelope proteins. In these cells, the gag, pol, and env genes can be derived from the same or different retroviruses.

A number of packaging cell lines suitable for the present invention are also available in the prior art. Examples of these cell lines include Crip, GPE86, PA317 and PG13 (see Miller et al., J. Virol. 65:2220-2224 (1991)). Examples of other packaging cell lines are described in Cone and Mulligan Proceedings of the National Academy of Sciences. USA, 81:6349-6353 (1984); Danos and Mulligan Proceedings of the National Academy of Sciences, USA, 85:6460-6464 (1988); Eglitis et al. (1988), supra; and Miller (1990), supra.

Packaging cell lines capable of producing retroviral vector particles with chimeric envelope proteins may be used. Alternatively, amphotropic or xenotropic envelope proteins, such as those produced by PA317 and GPX packaging cell lines may be used to package the retroviral vectors.

### C. Pharmaceutical Formulations

When used for pharmaceutical purposes, the nucleic acid encoding a polypeptide or oligonucleotide active agent is generally formulated in a suitable buffer, which can be any pharmaceutically acceptable buffer, such as phosphate buffered saline or sodium phosphate/sodium sulfate, Tris buffer, glycine buffer, sterile water, and other buffers known to the ordinarily skilled artisan such as those described by Good et al. Biochemistry 5:467 (1966).

The compositions can additionally include a stabilizer, enhancer or other pharmaceutically acceptable carriers or vehicles. A pharmaceutically acceptable carrier can contain a physiologically acceptable compound that acts, for example, to stabilize the nucleic acids of the invention and any associated vector. A physiologically acceptable compound can include, for example, carbohydrates, such as glucose, sucrose or dextrans, antioxidants, such as ascorbic acid or glutathione, chelating agents, low molecular weight proteins or other stabilizers or excipients. Other physiologically acceptable compounds include wetting agents, emulsifying agents, dispersing agents or preservatives, which are particularly useful for preventing the growth or action of microorganisms. Various preservatives are well known and include, for example, phenol and ascorbic acid. Examples of carriers, stabilizers or adjuvants can be found in Remington's Pharmaceutical Sciences, Mack Publishing Company, Philadelphia, PA, 17th ed. (1985).

### D. Administration of Formulations

The formulations containing a polynucleotide sequence encoding a polypeptide or oligonucleotide active agent can be delivered to target tissue or organ using any delivery method known to the ordinarily skilled artisan. In some embodiments of the invention, the encoding polynucleotide sequences are formulated for subcutaneous, intramuscular, intravenous, or intraperitoneal injection, or for oral ingestion or for topical application.

The formulations containing the nucleic acid of interest are typically directly administered to a cell. The cell can be provided as part of a tissue, such as red blood cells as a part of the circulatory system, or as an isolated cell, such as in tissue culture. The cell can be provided in vivo, ex vivo, or in vitro.

The formulations can be introduced into the tissue of interest in vivo or ex vivo by a variety of methods. In some embodiments of the invention, the nucleic acids of interest are introduced into cells by such methods as microinjection, calcium phosphate precipitation, liposome fusion, ultrasound, electroporation, or biolistics. In further embodiments, the nucleic acids are taken up directly by the target tissue or organ relevant to the disease or condition being treated, for example, when the targeted cells are the brain endothelial cells intracranial injection is appropriate.

In some embodiments of the invention, the nucleic acids of interest are administered ex vivo to cells or tissues explanted from a patient, then returned to the patient. Examples of ex vivo administration of therapeutic gene constructs include Nolta et al., Proc Natl. Acad. Sci. USA 93(6):2414-9 (1996); Koc et al., Seminars in Oncology 23(1):46-65 (1996); Raper et al., Annals of Surgery 223(2):116-26 (1996); Dalesandro et al., J. Thorac. Cardi. Surg., 11(2):416-22 (1996); and Makarov et al., Proc. Natl. Acad. Sci. USA 93(1):402-6 (1996).

Effective dosage of the formulations will vary depending on many different factors, including means of administration, target site, physiological state of the patient, and other medicines administered. Thus, treatment dosages will need to be titrated to optimize safety and efficacy. In determining the effective amount of the vector to be administered, the physician should evaluate the particular nucleic acid used, the disease state being diagnosed; the age, weight, and overall condition of the patient, circulating plasma levels, vector toxicities, progression of the disease, and the production of anti-vector antibodies. The size of the dose also will be determined by the existence, nature, and extent of any adverse side-effects that accompany the administration of a particular vector. For example, an antisense oligonucleotide in the amount of 1-1000, 10-200, or 20-100 mg can be delivered to a patient via intravenous injection at a frequency of weekly, bi-weekly, or monthly administration over at least one to three months or a longer time period. For CRISPR editing targeting the sST2 genomic region, as another example, each 5×10⁵ cells (e.g., hCMEC/D3 cells) are transfected with 0.5-50 μg; 1-20 μg; or 2-10 μg of a vector carrying genes encoding Cas9 together with a pair of sgRNAs. For CRISPR editing targeting the sST2 genomic region in human patients, the dose for lipid nanoparticles carrying sgRNAs and mRNA encoding Cas9 is in the range of 0.01-2; 0.02-1.0; 0.05-0.5; or 0.10-0.30 mg/kg of body weight delivered by i.v. injection 1-3 times over a period of 1-4 weeks.

### V. Kits

The invention also provides kits for treating Alzheimer's Disease or reducing risk of Alzheimer's Disease in a person in need thereof according to the method of the present invention. The kits typically include a container that contains (1) a pharmaceutical composition having an effective amount of one or more active agent capable of disrupting a genomic sequence encompassing rs1921622 and/or other genomic sites listed in Table 4 and Table 5 and/or suppressing mRNA transcribed from the genomic sequence; and (2) informational material containing instructions on how to dispense the pharmaceutical composition, including description of the type of patients who may be treated (e.g., human patients suffering from Alzheimer's Disease or at increased risk for the disease), the schedule (e.g., dose and frequency) and route of administration, and the like. In some cases, two or more containers are included in the kit to provide multiple pharmaceutical compositions each comprising an effective amount of at least one active agent, such as vector or vectors encoding components of a CRISPR system (e.g., a Cas9 nuclease or equivalent and one or more sgRNAs) or encoding an siRNA, a microRNA, a miniRNA, a lncRNA, or an antisense oligonucleotide targeting the genomic sequence encompassing rs1921622, including the 3′-UTR of the sST2 gene/transcript. Optionally, the kit may further comprise one or more additional containers, each containing at least one agent useful for sequencing at least a portion of the person's genome, especially the genomic sequence encompassing the genetic locus rs1921622 and/or other genomic sites listed in Table 4 and Table 5.

### EXAMPLES

The following examples are provided by way of illustration only and not by way of limitation. Those of skill in the art will readily recognize a variety of non-critical parameters that could be changed or modified to yield essentially similar results.

### Abstract

Changes in the levels of circulating proteins are associated with Alzheimer's disease (AD), whereas their pathogenic roles in AD are unclear. Here, we report that soluble ST2 (sST2), a soluble decoy receptor of IL-33/ST2 signaling, is involved in AD pathogenesis. We found that elevated sST2 levels are associated with more severe neurodegeneration and AD pathologic lesions in patients with AD, and can result in exacerbated AD accumulation and reduced A-microglia colocalization in an amyloidosis mouse model. Our genome-wide association study identified genetic variants in IL1RL1 (the gene that encodes sST2) that are associated with decreased sST2 levels. Typically, deletion of the rs1921622 variant by CRISPR/Cas9 genome editing, which is located in the enhancer element of IL1RL1, decreases the expression and secretion of sST2 in brain endothelial cells; that decreased sST2 level is associated with decreased risk of developing AD and less-severe AD-related endophenotypes in female APOE-ε4 carriers. Immunohistochemical and single-nucleus transcriptomic analyses of AD brains further suggest that in female APOE-ε4 carriers, rs1921622/lower sST2 levels exert protective effects by reducing AD accumulation through enhancing the activation of microglia and their colocalization with AD. Furthermore, disruption of the 3′-untranslated region (UTR) of sST2 transcript, either by deletion of genomic region of sST2 3′-UTR or administration of sST2 3′-UTR-targeting antisense oligomers (ASO), also decrease the gene and protein levels of sST2 ameliorated AD-related pathological changes in amyloidosis mouse models. Taken together, these findings demonstrate that sST2 is a novel disease-causing factor for AD, and decreasing sST2 level is a potential intervention strategies for the disease.

### Introduction

Alzheimer's disease (AD), the most common neurodegenerative disease and a leading cause of mortality in the elderly¹, is characterized by memory decline and cognitive impairment. Its pathological hallmarks include extracellular accumulation of amyloid-beta (AβD) peptides, which form Aβ plaques, and intracellular neurofibrillary tangles composed of hyperphosphorylated tau protein (P-tau)². In AD, Aβ acts as a danger-associated molecular pattern that triggers the activation of microglia, stimulating them to migrate towards and clear Aβ plaques through phagocytosis 3,. While the pathophysiological mechanisms underlying AD remain unclear, large-scale genome-wide association studies (GWASs) have revealed more than 40 AD-associated genes linked with microglial functions (e.g.. APOE, TREM2, BIN1, and CD33), suggesting that microglia play a key role in AD pathogenesis s. Notably, APOE-ε4, the strongest known risk factor for sporadic AD⁵, is suggested to mainly affect the Aβ accumulation in AD^{8,9}. The APOE gene contributes to the clearance of Aβthrough different cellular mechanisms; specifically, it promotes the clustering of microglia around Aβ, which subsequently degrade Aβ plaques^{6,10,11}. Postmortem single-cell/nucleus transcriptomic analyses of the AD brain have revealed a microglial subpopulation with increased APOE expression in AD^{12,13}. Moreover, in an amyloidosis mouse model, APOE is required to induce a specific microglial subpopulation that interacts with Aβ plaques⁶. Thus, microglial dysfunction is an essential causative factor of AD.

Besides genetic factors, changes in secreted signaling proteins in the brain milieu and in the circulation may disrupt microglial activities and contribute to the pathogenesis of AD^{14,15}. For example, the level of soluble TREM2 (sTREM2), a proteolytic cleavage product of the microglial receptor TREM2 that contains an extracellular domain, is increased in the cerebrospinal fluid (CSF) of patients with AD¹⁶⁻¹⁸. In a transgenic mouse model of amyloidosis, injection of sTREM2 alleviates Aβ accumulation and rescues memory deficits by enhancing the interaction between microglia and Aβ and hence subsequent Aβ phagocytosis^{19,20}. Moreover, the expression of a soluble form of VCAM1 (vascular cell adhesion molecule 1) cleaved from the full-length protein on endothelial cells is elevated in the plasma and CSF of patients with AD^{21,22}; this is suggested to mediate the reduced hippocampal neurogenesis and pro-inflammatory response of microglia during aging²³.

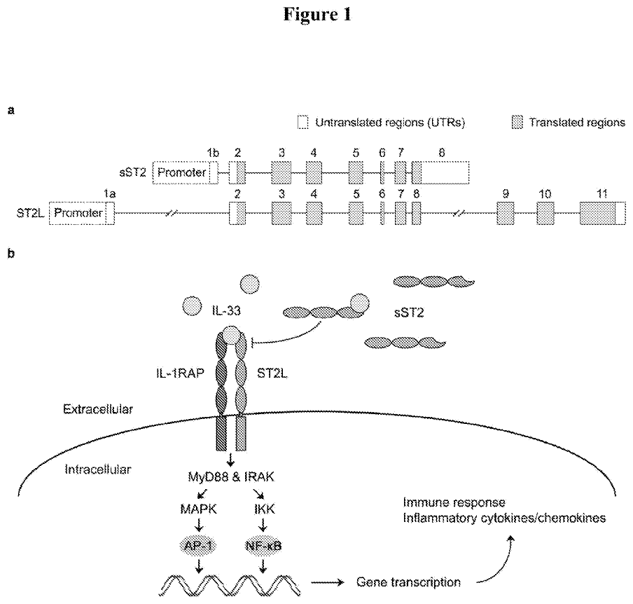

Other secreted forms of soluble receptors that can contribute to AD pathogenesis include soluble cytokine receptors comprising the ectodomains of membrane-bound cytokine receptors, which function as decoy receptors and attenuate cytokine-mediated signaling^{24,25}. In particular, soluble ST2 (sST2) is a secreted isoform of the receptor of the cytokine IL-33 (i.e.. ST2L [fill-length ST2]) produced by alternative promoter activation²⁶ (FIG. 1). ST2L is expressed by microglia in the brain²⁷, and activation of IL-33/ST2 signaling decreases Aβ accumulation and increases the Aβ-clearance capacity of microglia in transgenic mouse models of amyloidosis^{28,29}. Meanwhile, sST2 acts as a decoy receptor of IL-33 and can effectively inhibit IL-33/ST2 signaling^{30,31}. The altered sST2 level in plasma is observed in multiple inflammatory diseases, cancers, and cardiac diseases, being a promising biomarker of these diseases in the periphery³²⁻³⁵. Notably, recent evidence also shows that sST2 levels are elevated in the blood of patients with mild cognitive impairment or AD^{28,36}. Nonetheless, the regulatory mechanisms underlying the dysregulation of sST2 and whether sST2 has a pathological role in AD remain unclear.

Accordingly, in the present study, we investigated the roles of sST2 in AD pathogenesis and the regulation of sST2 expression and AD-associated pathologic changes by genetic factors. We showed that elevated sST2 levels impair microglial functions and exacerbate AD accumulation in a mouse model of amyloidosis. Moreover, we identified single nucleotide polymorphisms (SNPs) in IL1RL1 (the gene that encodes sST2 and ST2L), that are associated with lower plasma and CSF sST2 levels. Typically, genotype-expression association analysis and CRISPR/Cas9-based genome editing demonstrated that one of the sST2-associated SNPs, rs1921622, downregulates the gene expression and secretion of sST2 in human endothelial cells; and that female APOE-ε4 carriers who harbor this variant have a lower risk of AD and less-severe AD-associated endophenotypes. Subsequent single-nucleus transcriptomic profiling revealed that both the presence of rs1921622 and reduced sST2 level are associated with enhanced microglial activation in female patients with AD carrying APOE-ε4, indicating that sST2 levels modulate microglial activation status in AD. Concordantly, decreased sST2 level by disruption of sST2 3′-untranslated region (UTR) using antisense oligomers (ASOs) or genome editing attenuated AD-related pathological changes in amyloidosis mouse models. Taken together, our findings indicate that sST2 is a soluble factor in the brain milieu that plays disease-causing roles in AD pathogenesis, which can be a novel target for AD therapy, and decreasing sST2 levels could be a potential intervention strategy for the disease.

### Results

Levels of sST2 are Associated with Alzheimer's Disease and its Pathologic Changes

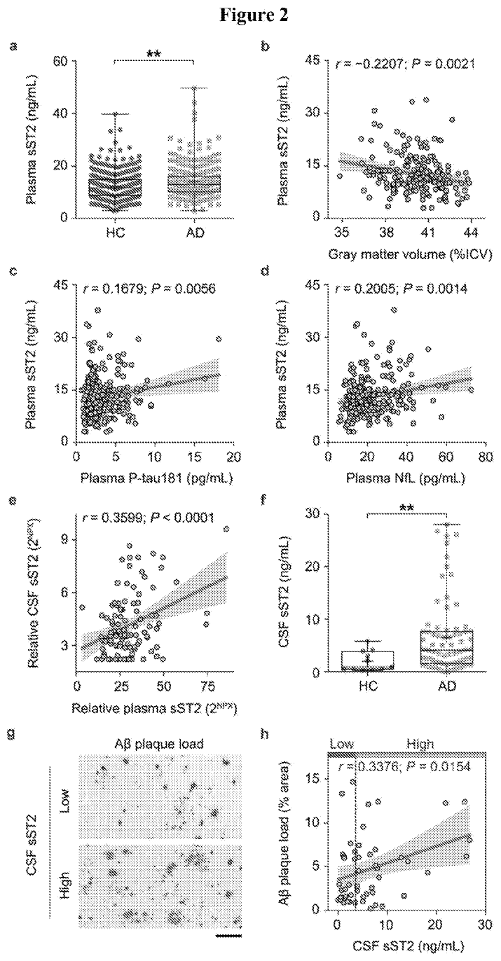

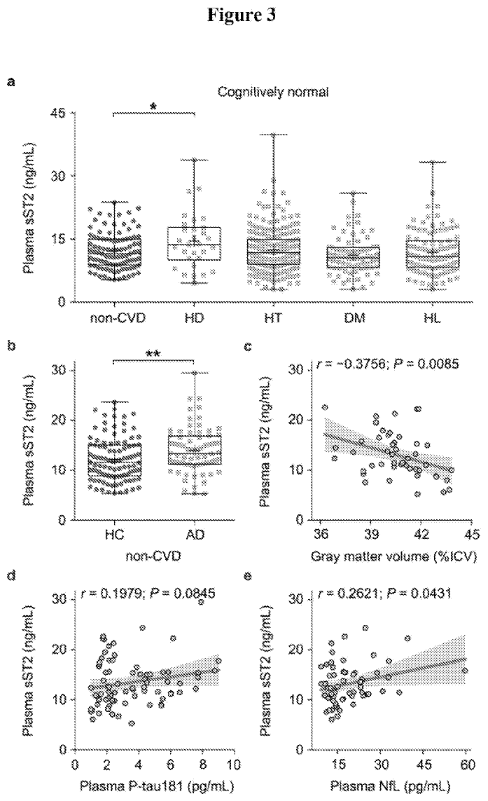

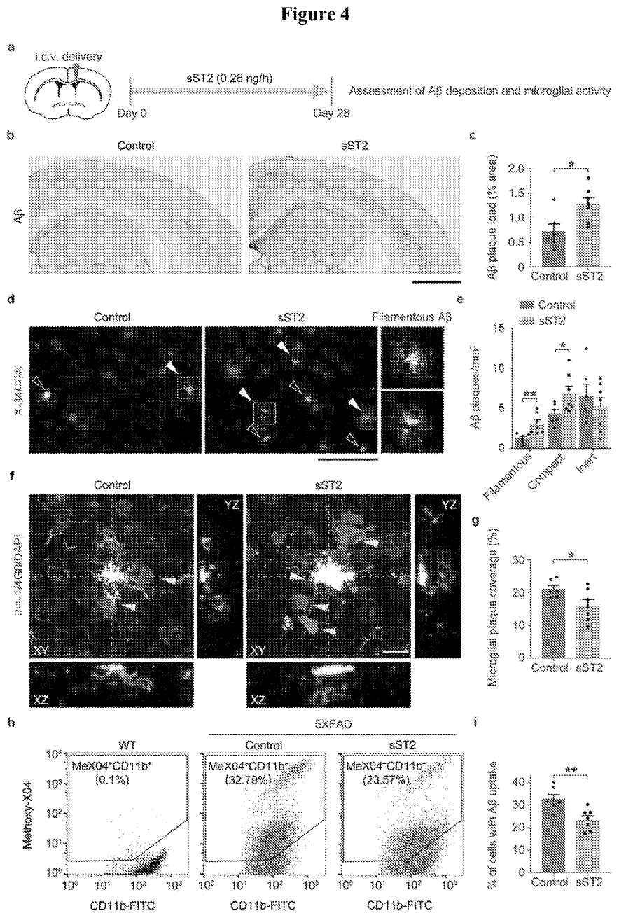

To investigate how sST2 participates in the pathogenesis of AD, we examined the associations between sST2 levels, and AD and its related endophenotypes. Specifically, we measured the plasma sST2 level of Chinese patients with AD and HCs recruited in Hong Kong (the “Chinese_cohort_l” hereafter; n=345 HCs and n=345 patients with AD; Table 2). We subsequently performed linear regression analysis between plasma sST2 level and AD, adjusting for age, sex, and population structure. The results show that plasma sST2 level was significantly higher in patients with AD than HCs (P<0.01; FIG. 2a). Moreover, given that sST2 is a well-known biomarker of cardiovascular diseases (CVDs)^{35,37} and was elevated in patients with heart disease in our cohort (P<0.05; FIG. 3a), we examined the changes of the plasma sST2 level in AD in a subcohort of participants without a clinical history of CVDs (termed “the non-CVD cohort”). Consistent with our findings in the overall cohort, within the non-CVD cohort, patients with AD again had a significantly higher plasma sST2 level than HCs (P<0.01; FIG. 3b), suggesting that the increase in plasma sST2 level in AD is independent of CVD status. In addition, plasma sST2 level was positively correlated with the AD-associated endophenotypes examined, namely gray matter atrophy and the levels of plasma biomarkers corresponding to neurodegeneration and AD (i.e.. P-tau181 and [NfL] neurofilament light polypeptide^{38,39}) in the overall cohort (FIG. 2b-d) and non-CVD cohort (FIG. 3c-e). These observations collectively suggest that plasma sST2 level is associated with AD and related endophenotypes.

Furthermore, we showed that plasma and CSF sST2 levels are positively correlated within the same individual (P<0.0001; n=107 participants from the ADRC cohort⁴⁰; FIG. 2e). Accordingly, we subsequently examined the association between CSF sST2 level and AD. Concordant with the regulation of the plasma sST2 level in AD (FIG. 2a), in the UK Brain Bank Network cohort (“UKBBN cohort” hereafter; n=11 HCs and n=75 patients with AD; Table 3), the CSF sST2 level was significantly higher in patients with AD than in HCs (P<0.01; FIG. 2e). Notably, in patients with AD, CSF sST2 level was positively correlated with Aβ plaque load in the frontal cortex (P<0.05; FIG. 2g, h). These findings indicate that circulating sST2 levels in both the blood and CSF are increased in AD and are associated with disease progression.

Increased sST2 Levels in the Brain Exacerbate Aβ Accumulation and Impair the Aβ-Clearance Capacity of Microglia

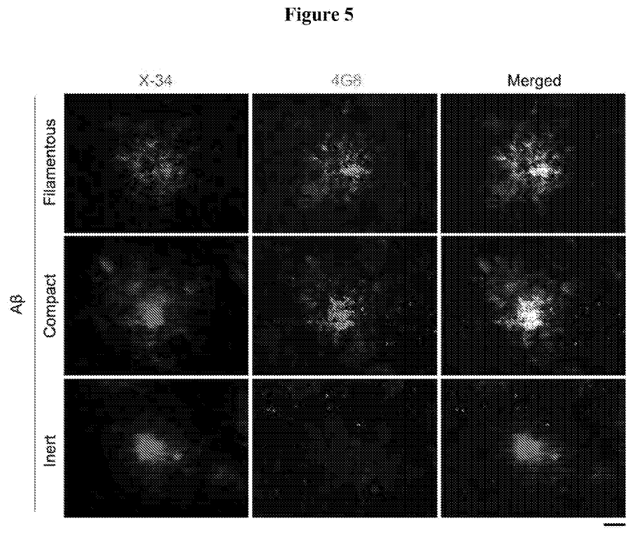

To investigate the causality between increased sST2 levels and AD progression, we examined the effects of increased sST2 levels on AD-associated pathologic changes in 5XFAD mice, a transgenic mouse model of amyloidosis. Consistent with our previous findings showing that activation of IL-33/ST2 signaling attenuates the pathologic features of AD, particularly Aβ accumulation^{28,29} continuous intracerebroventricular administration of recombinant mouse sST2 to 3-month-old 5XFAD mice for 28 days (FIG. 4a) significantly increased the Aβ plaque burden in the cortical regions compared to that in mice treated with vehicle control (P<0.05; FIG. 4b-c). In particular, the quantities of both the filamentous form of Aβ plaques (i.e., X-34⁺ diffuse fibrils without a dense core⁴¹) and the compact form of Aβ plaques (i.e., X-34⁺ dense cores with 4G8 halos) increased in sST2-treated mice (P<0.05; FIG. 4d, e and FIG. 5). However, sST2 administration did not affect the burden of less-toxic inert Aβ plaques (i.e., X-34⁺ dense cores without 4G8 labeling) in 5XFAD mice.

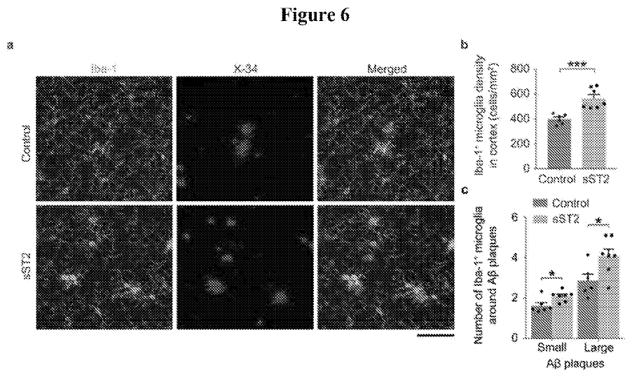

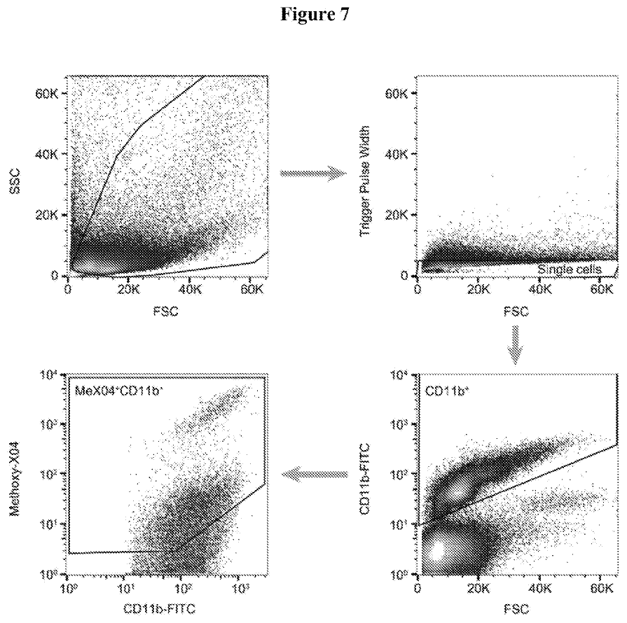

Given that microglia play a key role in Aβ clearance, we subsequently examined how sST2 regulates the interactions between microglia and Aβ plaques. In 5XFAD mice, sST2 injection increased the total number of microglia and the clustering of microglia around Aβ plaques in the cortical regions (P<0.05; FIG. 6). However, the coverage of Aβplaques by microglia in the cortical regions was lower in sST2-injected 5XFAD mice than in vehicle control-injected 5XFAD mice (FIG. 4f, g), suggesting that sST2 decreases the barrier formations of microglia around Aβ plaques. Moreover, flow cytometry analysis showed that sST2 administration decreased the Aβ-phagocytic capacity of microglia in 5XFAD mice as indicated by a decrease in the percentage of Aβ⁺ microglia (i.e., 23.57% CD11b⁺ and methoxy-X04⁺ cells in the sST2-treated group vs. 32.79% in the control group; P<0.01; FIG. 4h, I and FIG. 7). Thus, these findings collectively suggest that an increase in the sST2 levels in the brain exacerbates AD accumulation and impairs microglial AD-clearance capacity.

The Level of sST2 is Associated with a Genetic Variant of IL1RL1

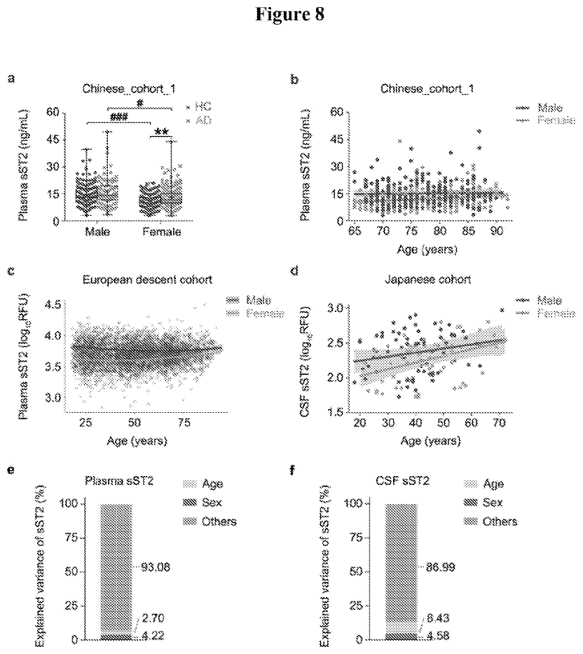

To understand how sST2 is regulated during the development of Aβ, we examined how various factors contribute to the changes of sST2 levels. Consistent with a previous study⁴², we found that plasma sST2 level was significantly associated with both age and sex in the Chinese population and populations of European descent; it was significantly lower in females than in males (P<0.001 in the Chinese_cohort_1, and the INTERVAL and LonGenity cohort⁴²) and increased significantly with age (P<0.001 in both cohorts; FIG. 8a-c). Moreover, CSF sST2 level was also associated with both age and sex in a Japanese cohort⁴³ (both P<0.01; FIG. 8d). However, when we examined the contributions of age, sex, and other factors to the variance in sST2 levels, age and sex together accounted for only 6.92% and 13.01% of the variance of plasma sST2 levels in the Chinese_cohort_1 and CSF sST2 levels in the Japanese cohort, respectively (Figure Se, f, suggesting that other factors modulate such changes.

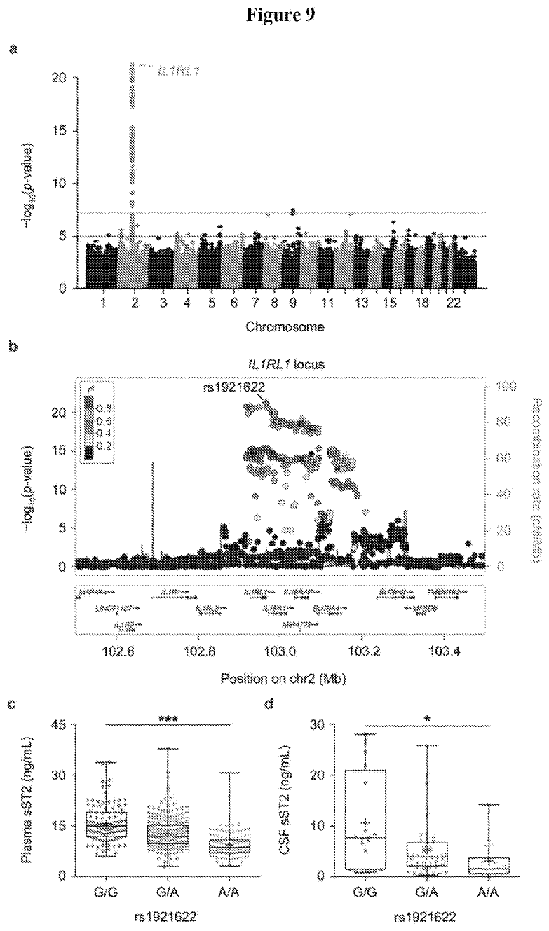

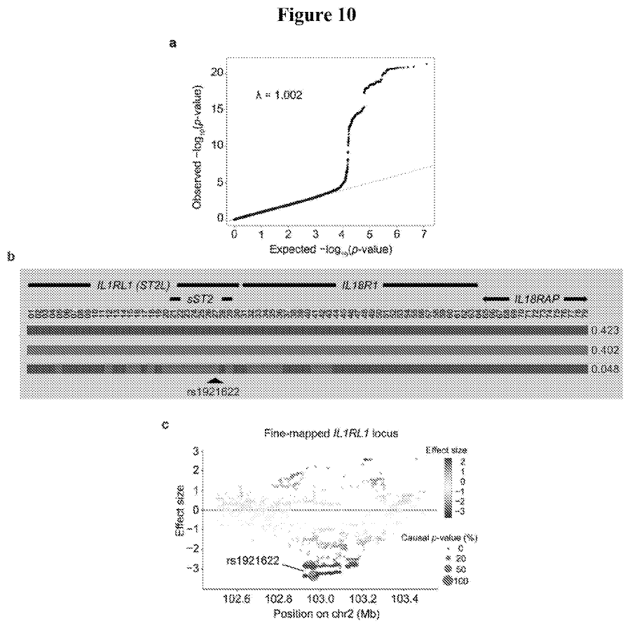

An association study in a population of European descent identified various genetic variants in the IL1RL1 gene that are associated with plasma sST2 level⁴⁴, suggesting that genetic factors contribute to the regulation of sST2 level. Nonetheless, given that these identified SNPs may form haplotype structures in this gene region⁴⁵, these SNPs might simply be inherited together with the causal variants. Therefore, to identify the key genetic regulator(s) of sST2, we utilized our whole-genome sequencing dataset⁴⁶ of Chinese_cohort_1 for the GWAS of plasma sST2 levels. Accordingly, we identified 575 genetic variants that were significantly associated with plasma sST2 level (P<1E-5) and found that these variants accounted for 54.86% of the variation thereof (FIG. 9a, FIG. 10a, and Table 4). Specifically, among these 575 variants, 79 genetic variants in or near IL1RL1 that form a haplotype were most strongly associated with sST2 level (FIG. 9b and FIG. 10b). Subsequent fine-mapping analysis through CAVIAR⁴⁷ identified the sentinel variant rs1921622 (G/A) as the putative causal variant (with 99.9% probability) that modulates the plasma sST2 level (FIG. 10c and Table 5). In the Chinese_cohort_1, the rs1921622 A allele was associated with a 20% decrease in plasma sST2 level in an allele dose-dependent manner (P<0.001; FIG. 9c). Moreover, the CSF sST2 level was consistently lower in carriers of the rs1921622 A allele than in noncarriers in the UKBBN cohort (P<0.05; FIG. 9d). Notably, rs1921622 alone accounted for 18.04% and 18.29% of the variance in plasma and CSF sST2 levels, respectively, which is much greater than contributions of age and sex. Hence, our fine-mapping analysis using whole-genome sequencing data identified rs1921622 as a key genetic factor that modulates the plasma and CSF levels of sST2.

The Rs1921622 Locus Regulates sST2 Expression in Human Brain Endothelial Cells

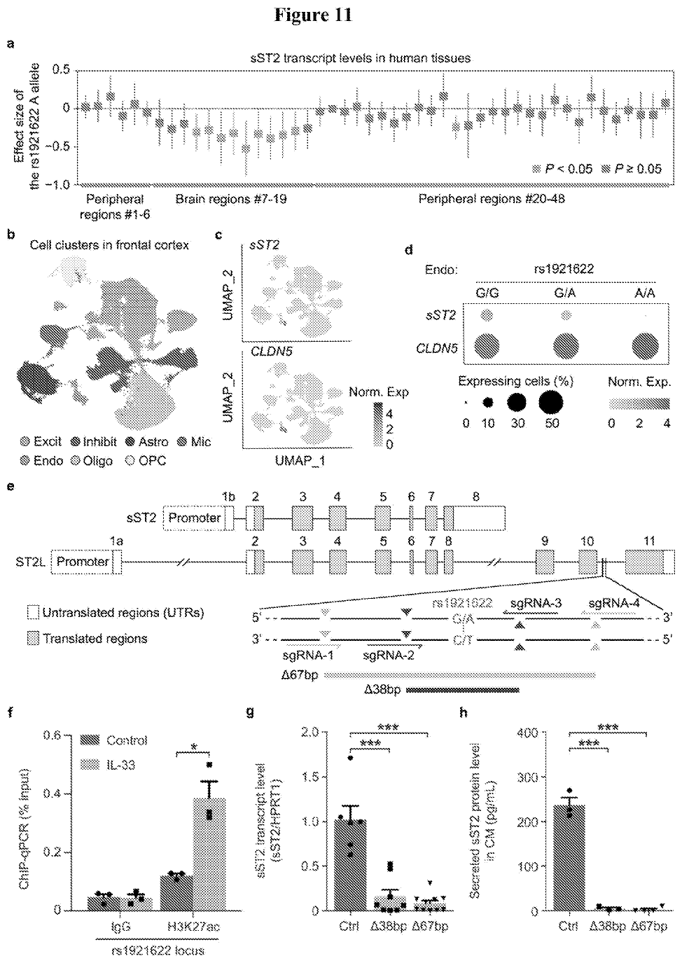

As rs1921622 is a noncoding variant located in the intronic region of ST2L, which is downstream of the region encoding sST2, we examined whether it modulates expression of sST2 and ST2L transcripts. Genotype-expression association analysis using a Genotype-Tissue Expression (GTEx) dataset^{48,49} showed that compared to noncarriers, individuals carrying the rs1921622 A allele exhibited a significantly lower transcript level of sST2 but not ST2L in multiple brain regions (e.g., the nucleus accumbens, amygdala, hippocampus, and frontal cortex; P<0.05; FIG. 1a and Table 1). Furthermore, analysis of our previously released human frontal cortex single-nucleus RNA sequencing (snRNA-seq) dataset⁵⁰ of the UKBBN cohort revealed that sST2 is exclusively expressed by endothelial cells (i.e.. CLDN5-expressing cells) in the frontal cortex (FIG. 11b, c). In addition, cell-type-specific genotype-expression association analysis showed that compared to individuals carrying the major allele, those carrying the rs1921622 A allele had a lower endothelial cell sST2 transcript level (P<0.01) and fewer sST2-expressing endothelial cells in an allele dose-dependent manner (P<0.05; FIG. 11d). These results collectively indicate that the rs1921622 variant is associated with decreased sST2 expression in brain endothelial cells in humans.

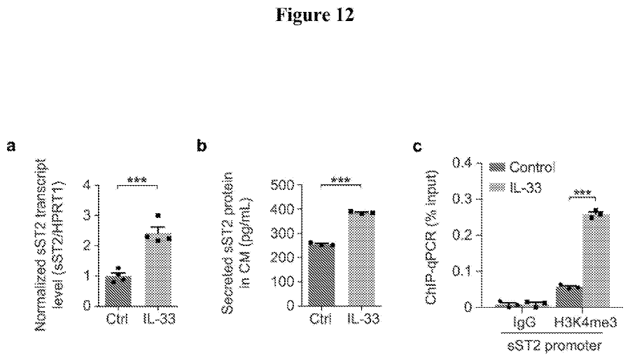

To investigate the role of rs1921622 in the decreased sST2 expression in endothelial cells, we examined whether rs1921622 and the surrounding genomic region (FIG. 11e) regulate sST2 transcription. Given that noncoding variants commonly modulate gene expression by functioning as enhancer elements⁵¹, we first examined enhancer activity at the rs1921622 locus in a human cerebral microvascular endothelial cell line, hCMEC/D3 cells. Administration of the cytokine IL-33 (the ligand of ST2L) increased the expression and secretion of sST2 in hCMEC/D3 cells (both P<0.001; FIG. 12a, b). Moreover, chromatin immunoprecipitation (ChIP) assay showed that these IL-33-treated hCMEC/D3 cells exhibited increased occupancy of an active enhancer histone mark (i.e.. H3K27ac) at the rs1921622 locus with a concomitant higher level of H3K4me3 histone modification (which indicates active promoter regions) at the sST2 promoter region (both P<0.05; FIG. 11f and FIG. 12c). Hence, these results indicate that rs1921622 is located at a potential enhancer element of the sST2 gene.

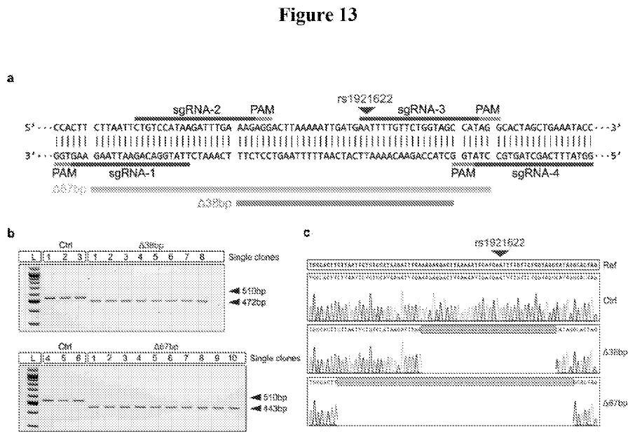

To further demonstrate that the region harboring rs1921622 contributes to regulation of sST2 expression, we used a CRISPR/Cas9-based approach to delete the genomic region harboring the rs1921622 locus in hCMEC/D3 cells. Accordingly, we generated 2 different hCMEC/D3 cell lines with biallelic 38-bp and 67-bp deletions (designated Δ38 bp and Δ67 bp, respectively) encompassing the rs1921622 locus (FIG. 11e and FIG. 13). Notably, loss of the 38 or 67 bp flanking the rs1921622 locus significantly decreased the sST2 transcript level in hCMEC/D3 cells (P<0.001; FIG. 11g) and concomitantly abolished sST2 protein secretion (P<0.001; FIG. 11h). These results collectively indicate that the rs1921622-containing region has a putative regulatory role as an enhancer element that regulates sST2 expression in endothelial cells.

### The Rs1921622 a Allele Protects Against Alzheimer's Disease in APOE-ε4 Carriers

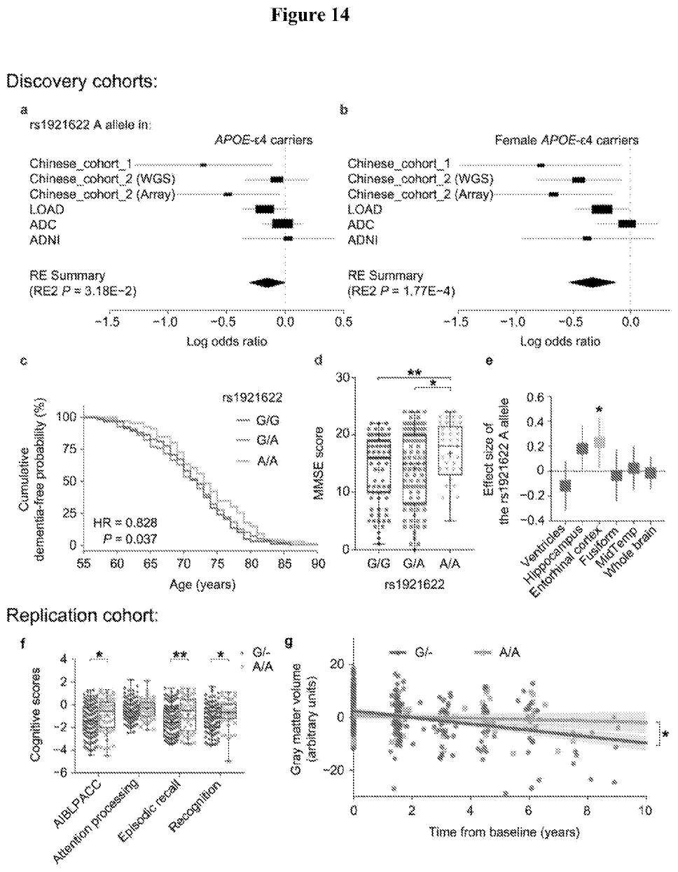

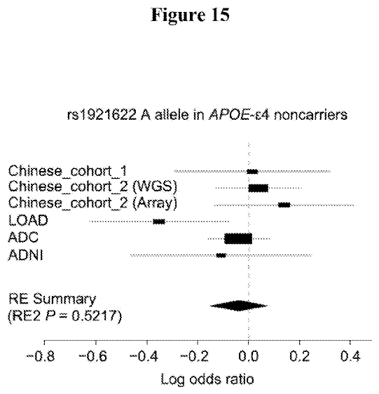

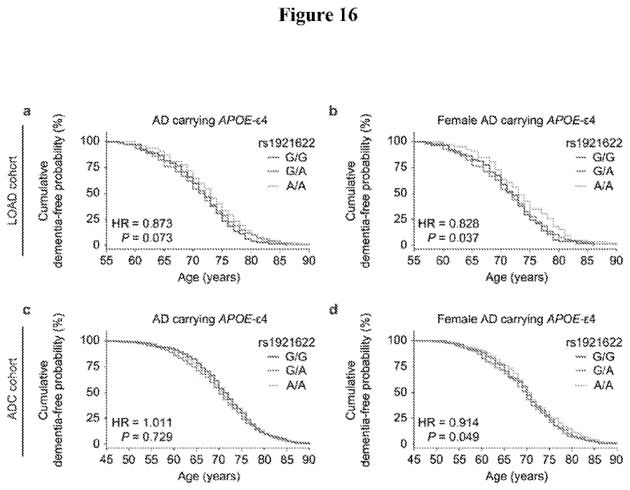

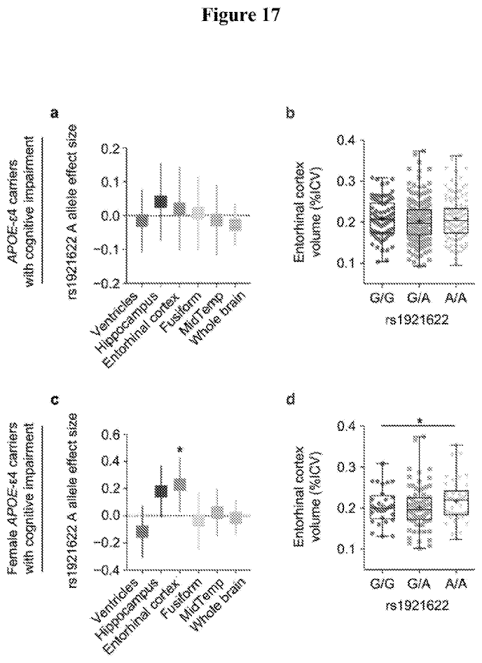

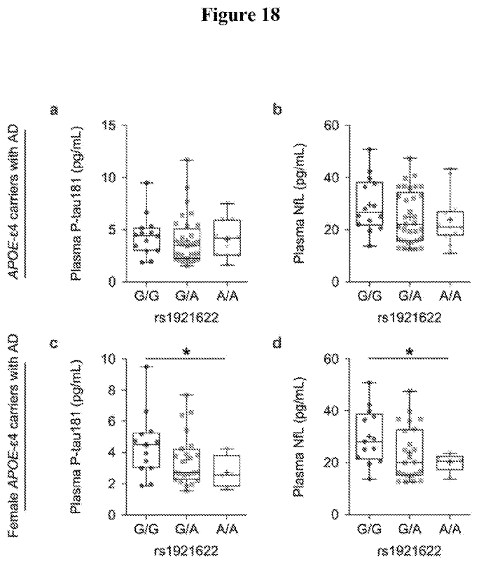

Given our findings that circulating sST2 levels are associated with AD risk and related endophenotypes, we examined whether the rs1921622 A allele is associated with decreased AD risk. Accordingly, we used the following 6 independent AD datasets as the discovery cohorts: the Chinese_cohort_1 dataset; the WGS and array datasets of Chinese_cohort_2⁵²; and 3 public datasets from populations of European descent (i.e., the Late-Onset Alzheimer's Disease [LOAD]⁵³, Alzheimer's Disease Center [ADC]^{54,55}, and Alzheimer's Disease Neuroimaging Initiative [ADNI] datasets) (N=5,436 HCs and N=5,556 patients with AD; Tables 2, 6). Genotype-phenotype association analysis showed that the rs1921622 A allele was weakly associated with AD risk in all subjects (odds ratio [OR]=0.945, Han and Eskin's random effects model [RE2]=8.90E-2; Table 7). Nonetheless, we observed a significant genetic interaction between the rs1921622 A allele and APOE-ε4 (P=0.0046): the rs1921622 A allele exerted significant protective effects against AD risk in APOE-ε4 carriers (OR=0.860, RE2=3.18E-2; FIG. 14a and Table 7), and this protective effect was absent in APOE-ε4 noncarriers (OR=0.964, RE2=5.22E-1; FIG. 15). Specifically, the rs1921622 A allele had a more prominent protective effect against AD risk in female APOE-ε4 carriers than male APOE-ε4 carriers (OR=0.717, RE2=1.77E-4; FIG. 14b and Table 8). Moreover, regarding AD-associated endophenotypes, although the protective effects of the rs1921622 variant were not obvious in overall APOE-ε4 carriers with AD, they were significant in female APOE-ε4 carriers with AD, including delayed onset of dementia, better cognitive scores, decreased atrophy of the entorhinal cortex, and decreased neurodegeneration (indicated by plasma levels of P-tau181 and NfL) (all P<0.05; FIG. 14c-e and FIGS. 16-18).

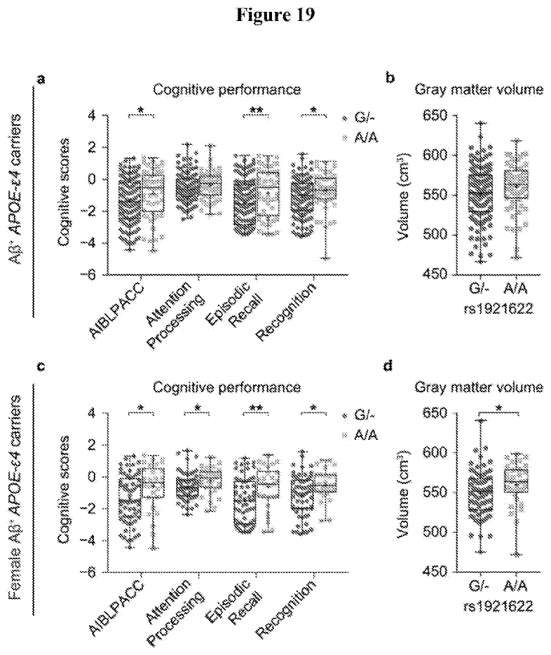

To confirm the protective effects of the rs1921622 A allele against AD, we examined AD-associated endophenotypes in an independent replication cohort: the Australian Imaging, Biomarkers and Lifestyle cohort (AIBL; n=190), in which the Aβ deposition (i.e.. Aβ⁺) in the brains of participants was confirmed by positron emission tomography⁵⁶. Concordant with the findings from the discovery cohorts, among Aβ⁺ subjects harboring the APOE-ε4 allele, the presence of the rs1921622 A allele was associated with improved cognitive performance as indicated by AIBL Preclinical Alzheimer Cognitive Composite (AIBLPACC) score and cognitive subprocesses including episodic recall and recognition (all P<0.05; FIG. 14f. Moreover, the rs1921622 A allele exerted stronger AD protective effects in female Aβ⁺ APOE-ε4 carriers than in overall Aβ⁺ APOE-ε4 carriers as indicated by the associations between the rs1921622 A allele and endophenotypes including attention processing ability and gray matter volume (both P<0.05; FIG. 19). Importantly, in a subgroup of 183 Aβ⁺ APOE-ε4 carriers from this cohort whose gray matter volume was traced for 10 years, the progression of gray matter atrophy was slower among those carrying the rs1921622 A allele than that in noncarriers (P<0.05; FIG. 14g). Thus, these results validate the protective effects of the rs1921622 A allele against cognitive decline and gray matter atrophy among APOE-ε4 carriers in an independent Aβ⁺ cohort.

Harboring of the Rs1921622 a Allele in Female APOE-ε4 Carriers with Alzheimer's Disease is Associated with Decreased Aβ Deposition and Increased Microglial Activation

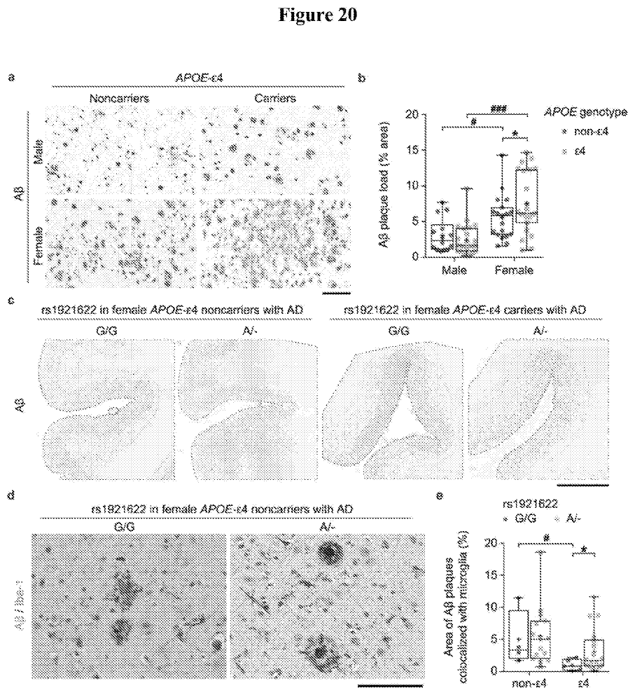

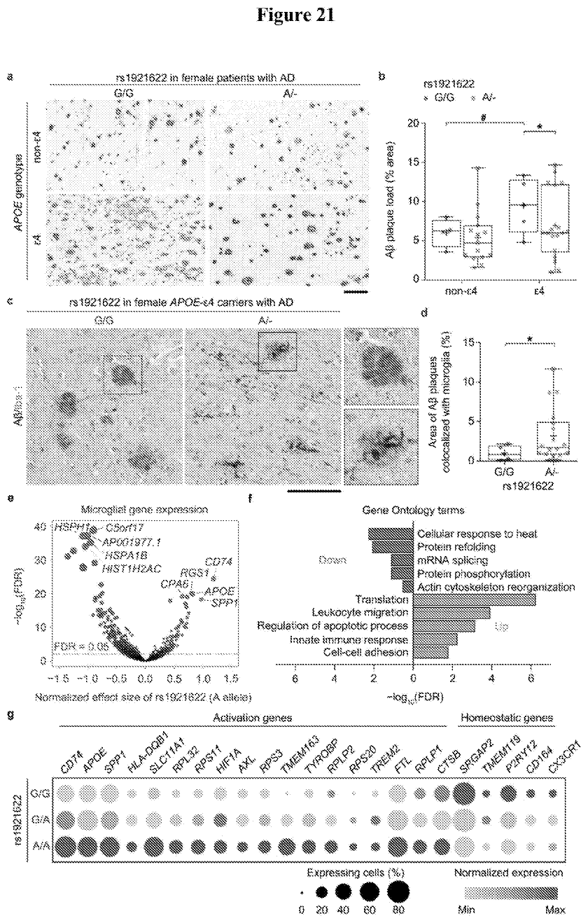

Given our findings that the rs1921622 A allele exerts AD-protective effects in APOE-ε4 carriers, specifically females, we subsequently examined whether this protective variant modulates AD deposition in postmortem human brains. Among patients with AD, female but not male APOE-ε4 carriers exhibited greater AD deposition in the frontal cortex than APOE-ε4 noncarriers (P<0.05; FIG. 20a, b). However, upon further stratification according to rs1921622 genotype, female APOE-ε4 carriers harboring the rs1921622 A allele exhibited significantly less AD deposition than those without this allele (P<0.05; FIG. 21a, b and FIG. 20c), suggesting that the rs1921622 A allele attenuates the effects of APOE-ε4 on AD-related pathologic changes. While immunohistochemical analysis revealed that female APOE-ε4 carriers with AD exhibited less microglial coverage of Aβ plaques (i.e., decreased proportion of Aβcolocalized with Iba-1⁺ microglia) than that in APOE-ε4 noncarriers (P<0.05; FIG. 20d, e), those harboring the rs1921622 A allele exhibited significantly increased colocalization between Iba-1⁺ microglia and Aβ plaques (P<0.05; FIG. 21c, d). Thus, these results suggest that in female APOE-ε4 carriers with AD, the rs1921622 A allele is associated with enhanced microglia-AP interaction and decreased AD pathologic lesions.

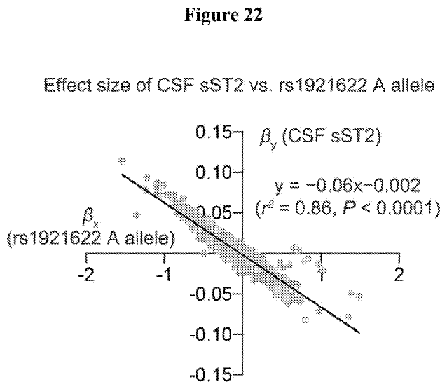

Next, to investigate the regulatory effects of the rs1921622 variant on microglial activities at the molecular level, we conducted an association analysis with a microglial snRNA-seq dataset in the frontal cortices⁵⁰ of female APOE-ε4 carriers with AD from the UKBBN cohort. There was a strong negative correlation between the effects of the rs1921622 A allele and the effects of CSF sST2 level on microglial gene expression (P<0.0001; FIG. 22), supporting the notion that the protective rs1921622 variant exerts its modulatory effects on microglia through the regulation of CSF sST2 level. Specifically, we identified 1,696 microglial genes that were significantly associated with rs1921622 genotype: there were 445 and 1,251 genes whose expression was upregulated and downregulated, respectively, in patients harboring the rs1921622 A allele compared to those without the allele (false discovery rate [FDR]-adjusted P<0.05; FIG. 21e). Moreover, Gene Ontology (GO) analysis showed that among the microglial genes associated with the rs1921622 A allele, those with upregulated expression are associated with leukocyte migration (FDR-adjusted P=1.2E-4) and innate immune response (FDR-adjusted P=5.4E-3), whereas those with downregulated expression are mainly involved in protein refolding (FDR-adjusted P=8.1E-3) or mRNA splicing (FDR-adjusted P=7.7E-2) (FIG. 21f).

Recent studies of microglia in the mouse and human brains revealed a subset of “microglial activation genes,” including CD74, APOE, and TREM2, whose expression levels upregulated in the context of Aβ1,3 and are involved in AD phagocytosis by microglia^{6,57,58}. Therefore, we investigated whether these genes are associated with the rs1921622 A allele. Interestingly, among female APOE-ε4 carriers with AD, the rs1921622 A allele was associated with increased expression of these microglial activation genes-specifically increased transcript levels of CD74, APOE, and TREM2 in microglia as well as an increased proportion of TMEM163⁺ microglia—in an allele dose-dependent manner (FIG. 21g). In contrast, the rs1921622 A allele was also associated with decreased expression of homeostatic genes including SRGAP2, TMEM119, and P2RY12, which commonly indicate a less-reactive microglial state^{6,57} (FIG. 21g). Therefore, these results collectively indicate that the rs1921622 A allele promotes the transition of microglia to a more activated state in female APOE-ε4 carriers with AD.

Disruption of the 3′Untranslated Region of sST2 Reduces sST2 Level and Alleviates Amyloid Associated Pathologies in 5XFAD Mice

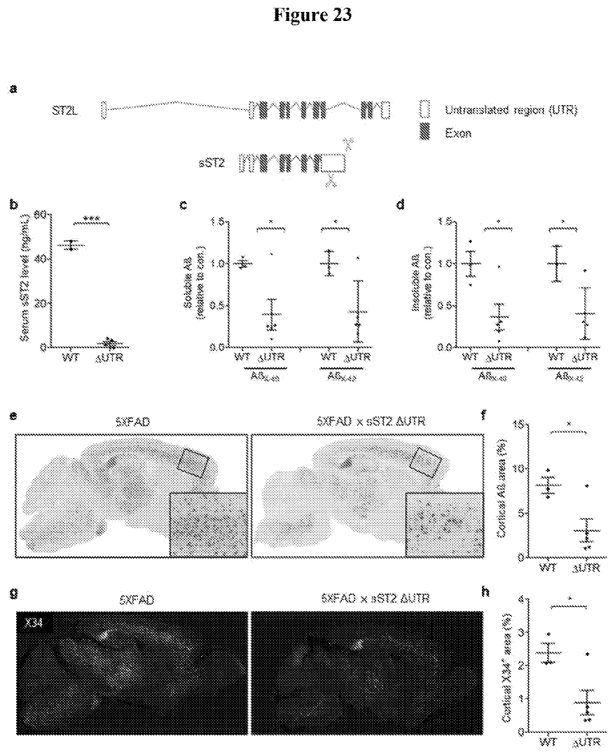

To establish more generalized methods to reduce sST2 level and ameliorate AD-related genotypes, we searched for an alternative solution to manipulate sST2 level without affecting the expression of ST2L. While sST2 and ST2L share most of the coding sequence, the 3′untranslated region (UTR) of sST2 transcript is unique (FIG. 23a). Therefore, 3′UTR region of sST2 could be the perfect target to manipulate sST2 level. With the generation of the mouse model with deletion of sST2, we found that deletion of 3′UTR region of sST2 significantly reduced the serum sST2 level (FIG. 23b). Moreover, deletion of sST2 3′UTR also reduced the cortical Aβ level in both soluble and insoluble contents in the 6-month-old amyloidogenesis mouse model, 5XFAD mice (FIG. 23c,d). Deletion of the 3′UTR of sST2 also leaded to the decrease of Amyloid plaque burden (FIG. 23e-h). Taken together, 3′UTR region might be a good target to reduce sST2 level and AD-associated pathologies.

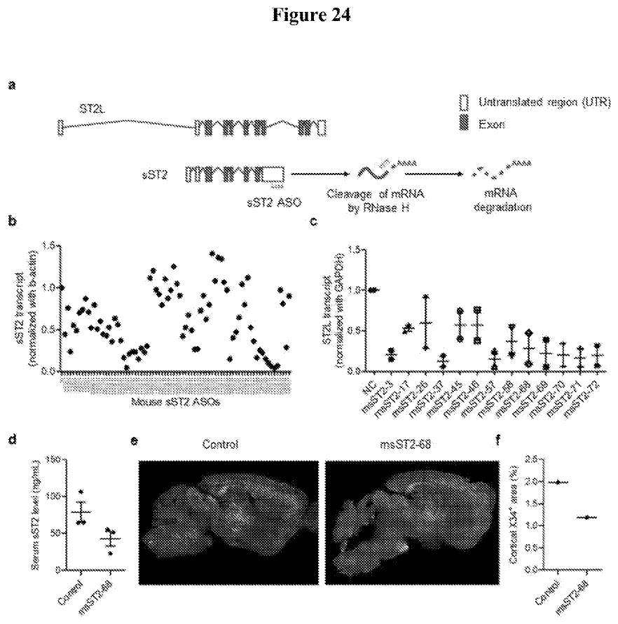

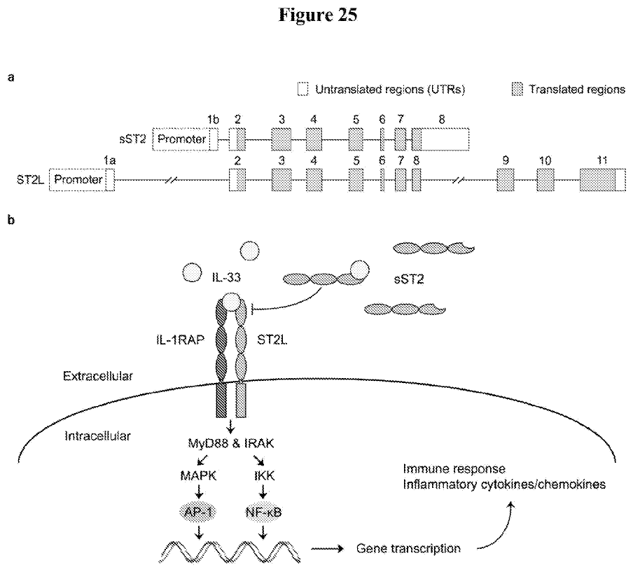

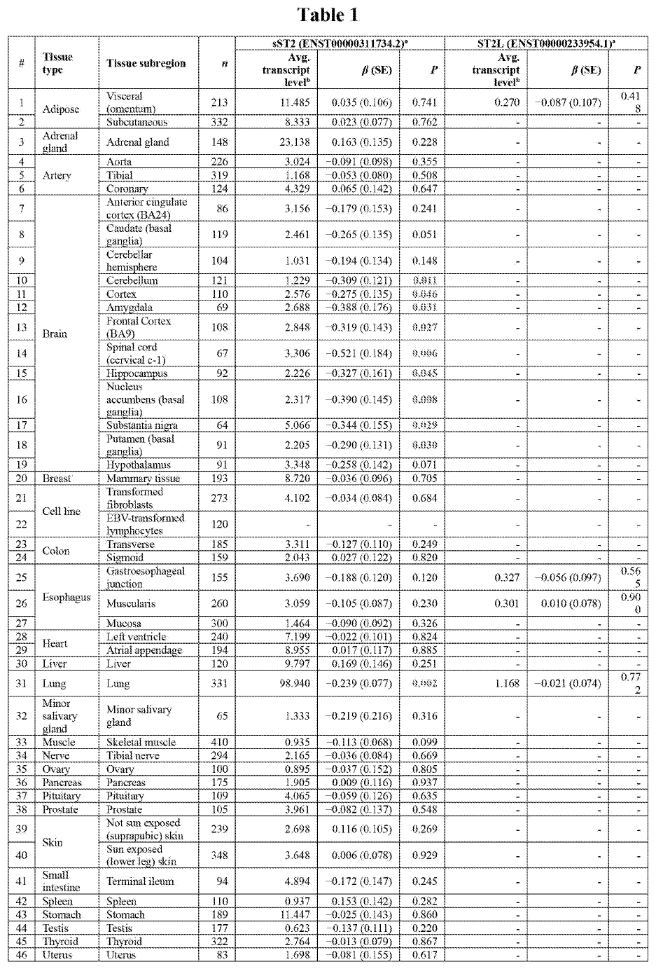

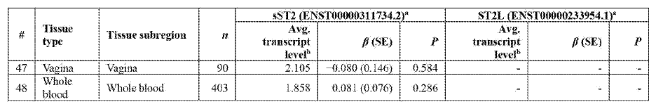

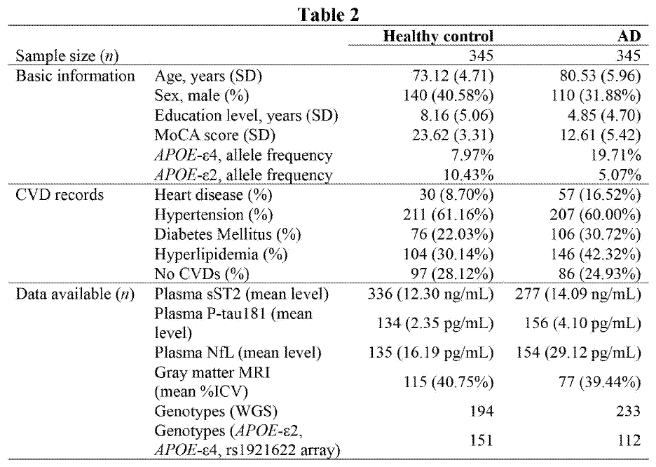

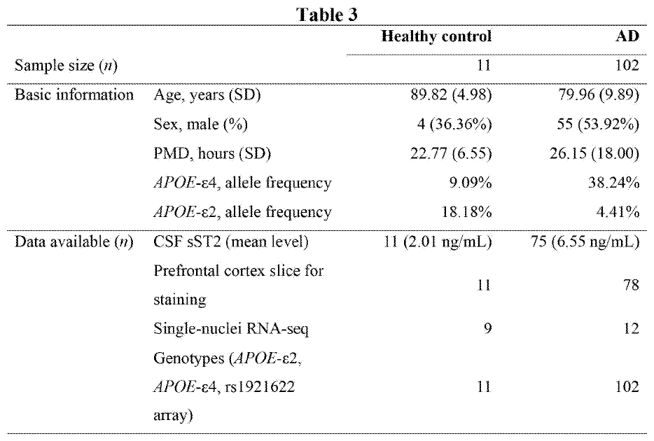

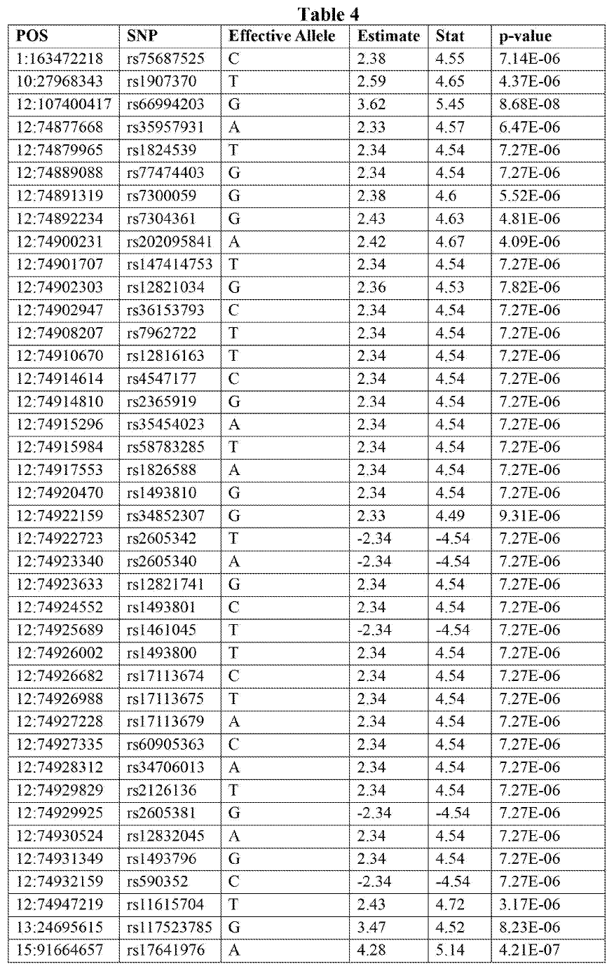

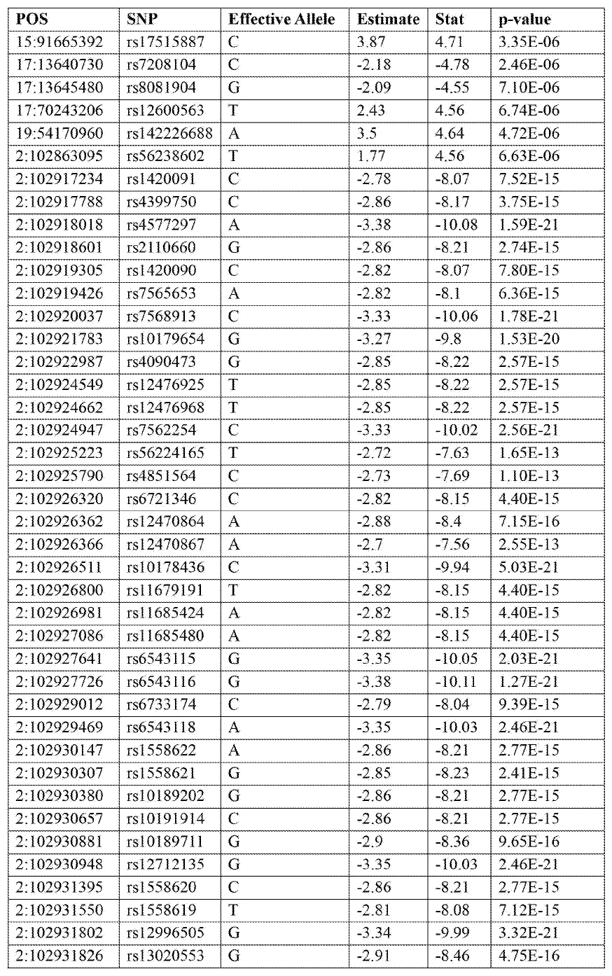

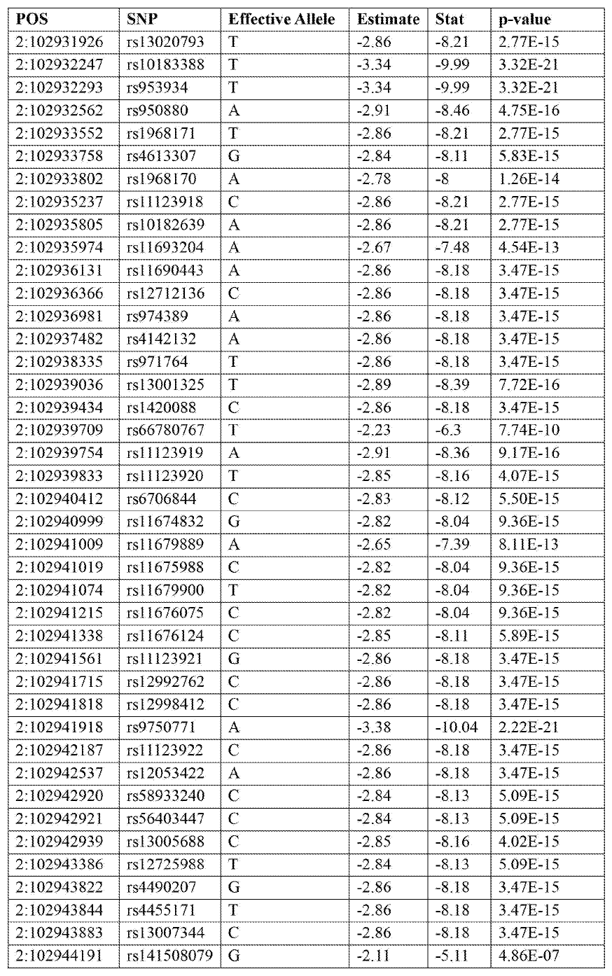

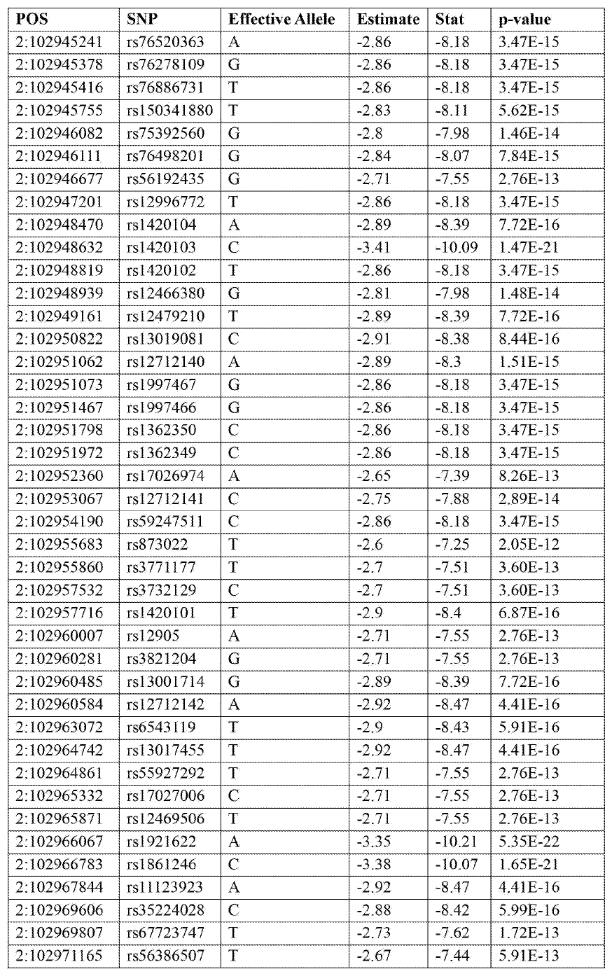

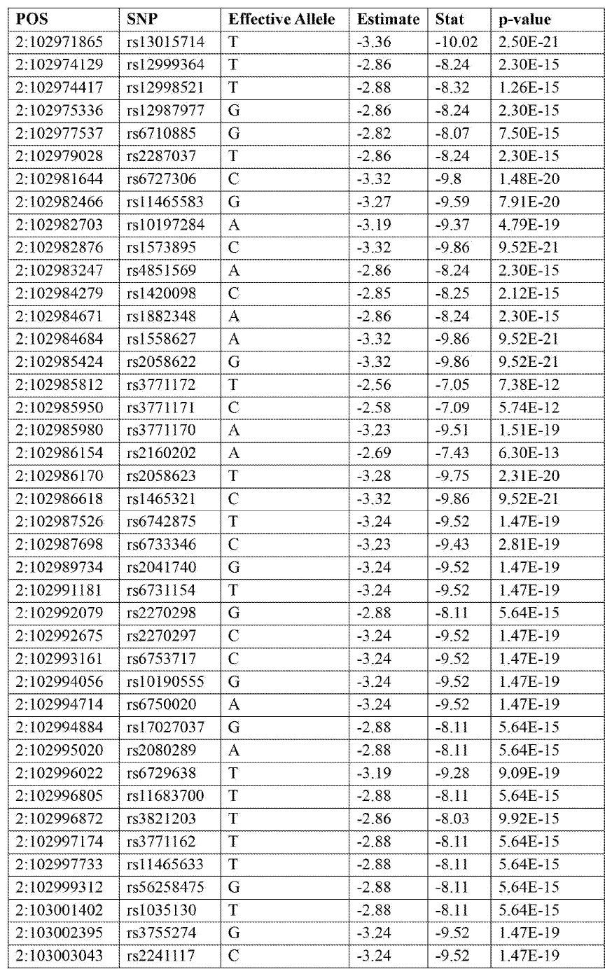

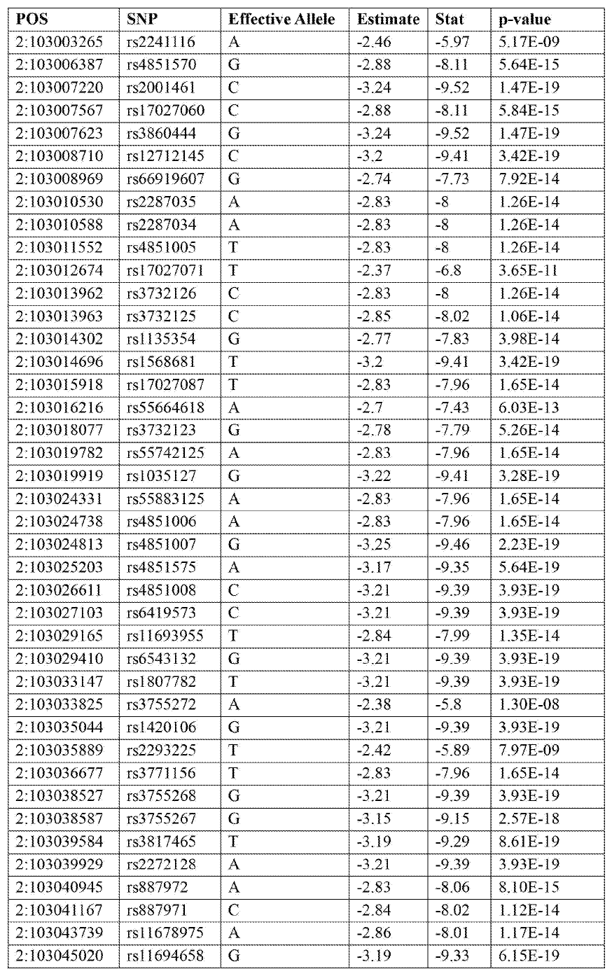

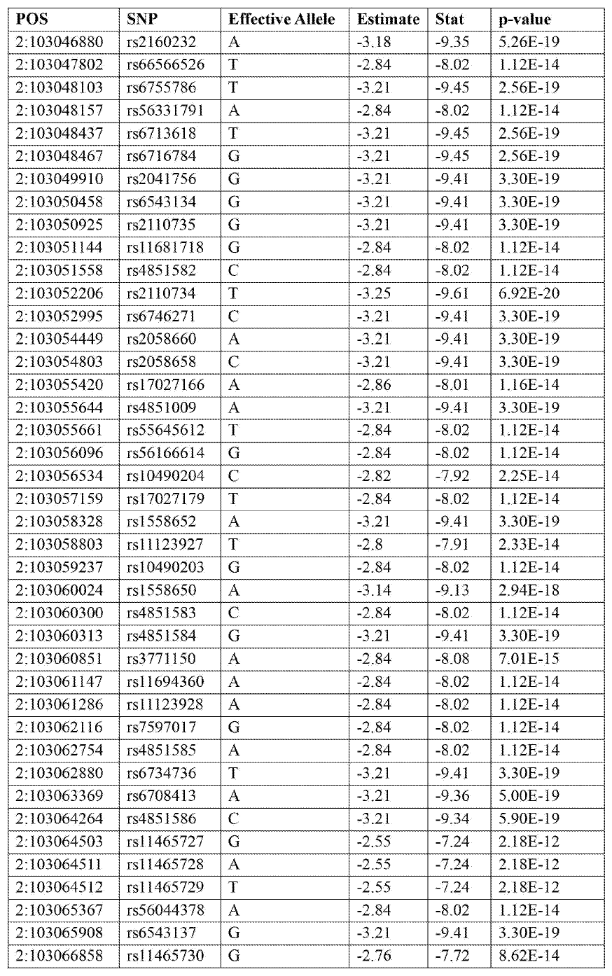

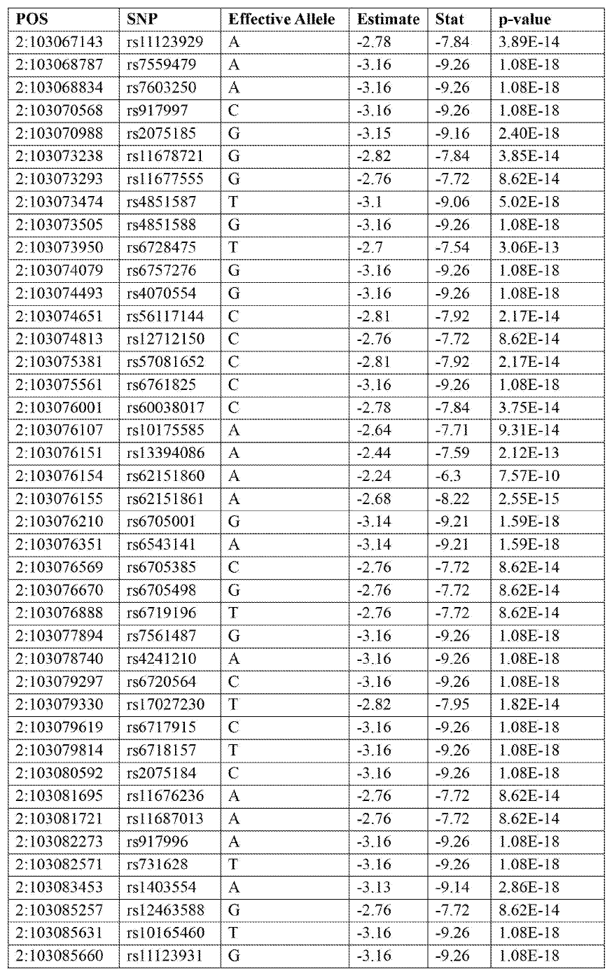

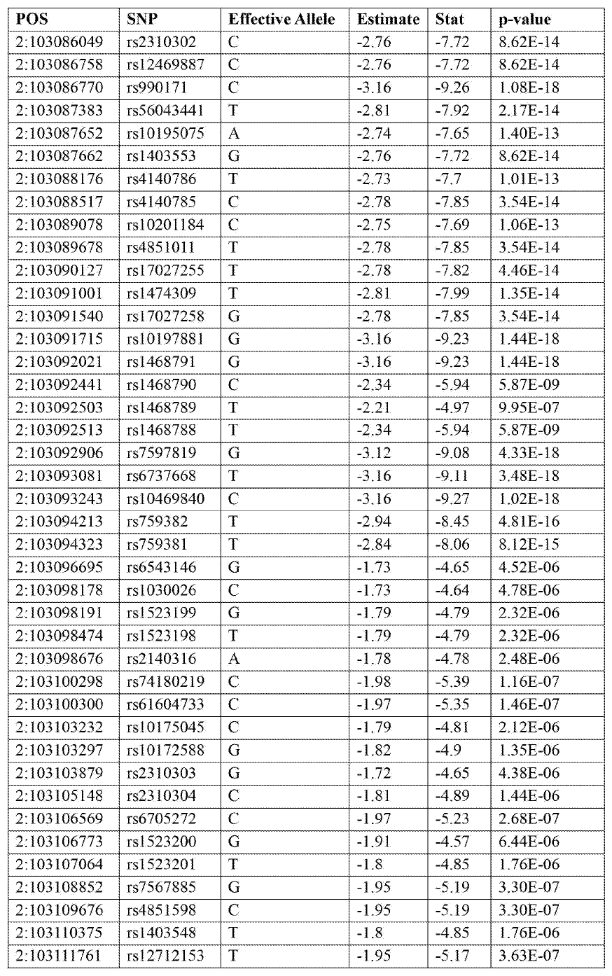

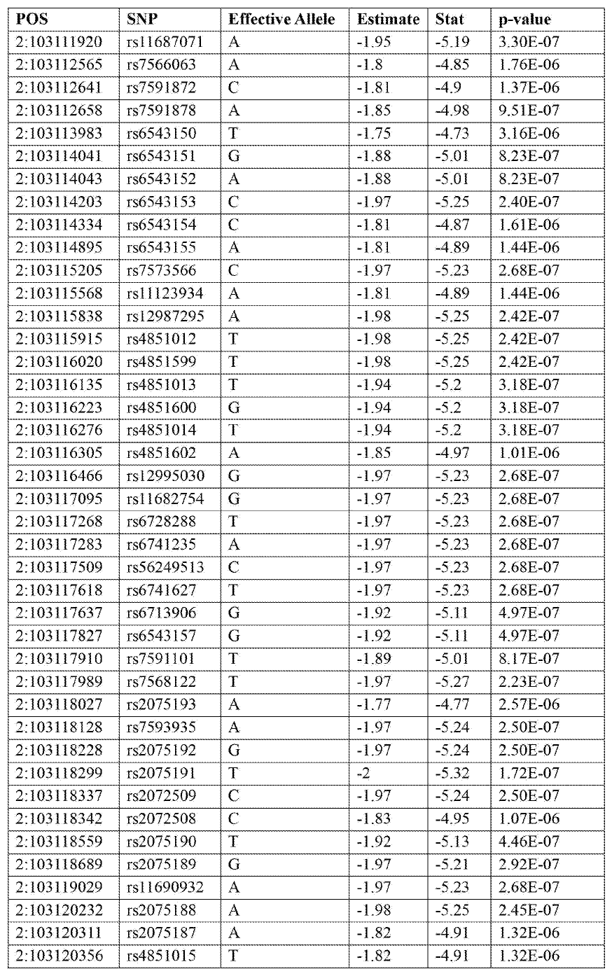

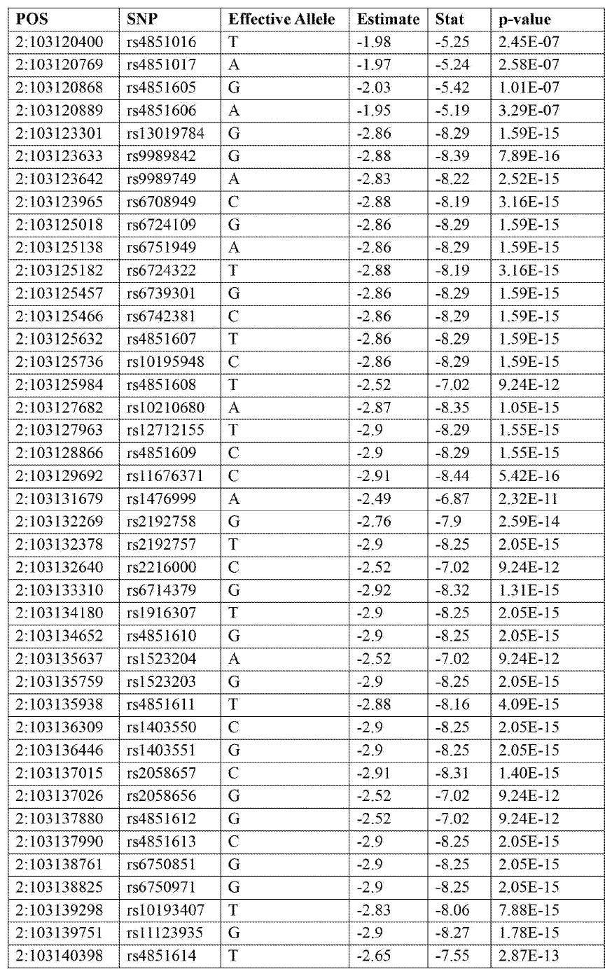

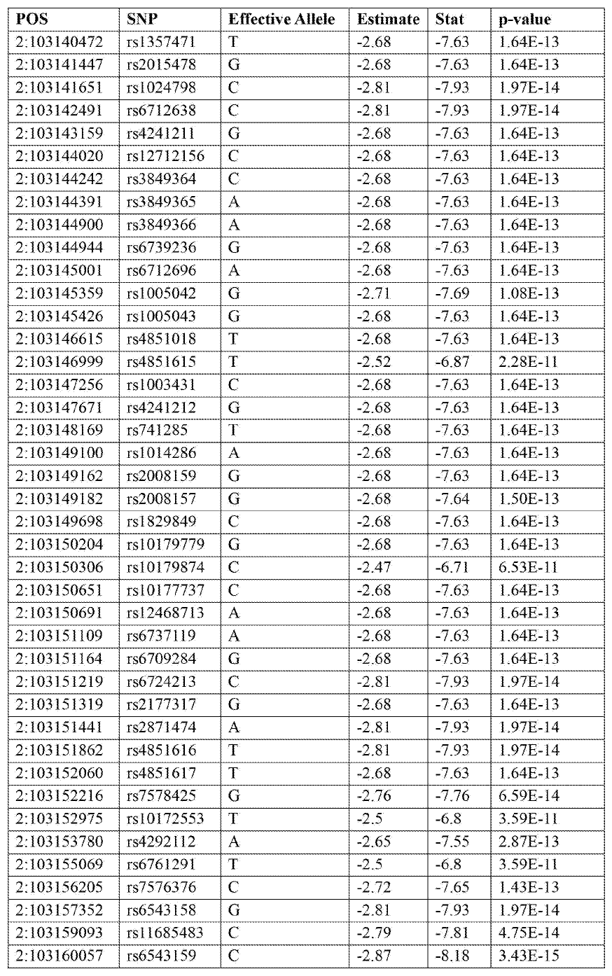

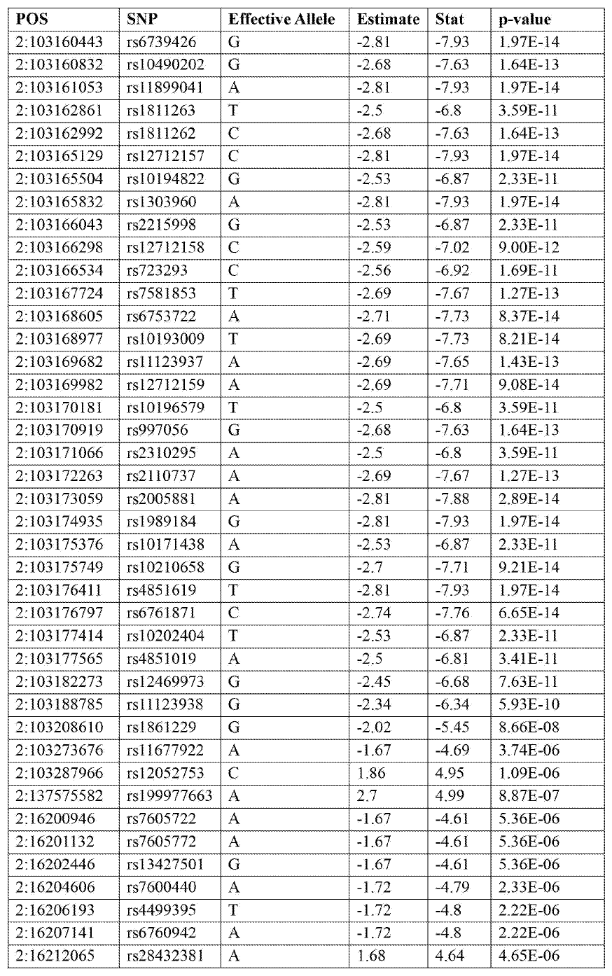

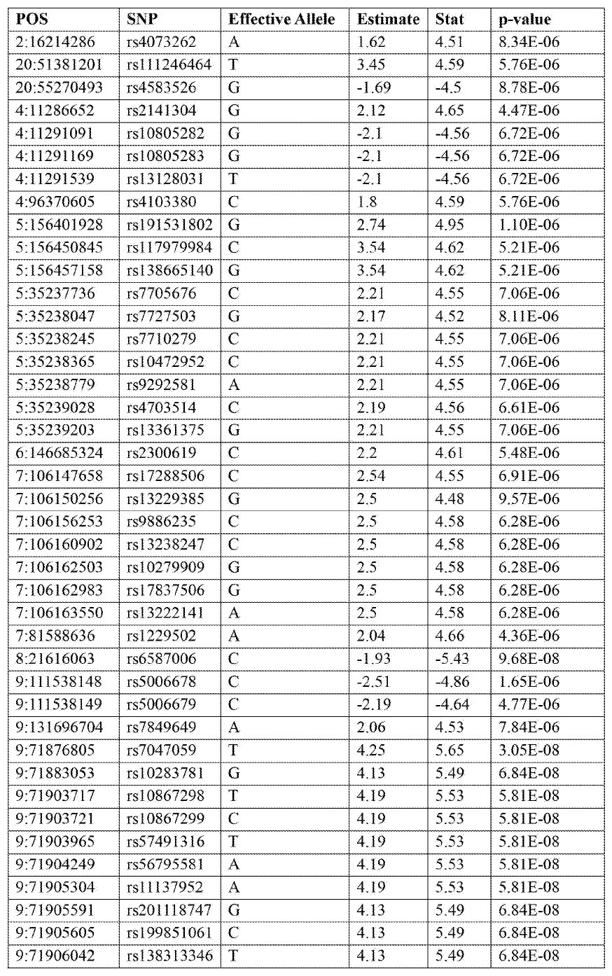

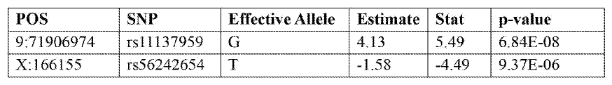

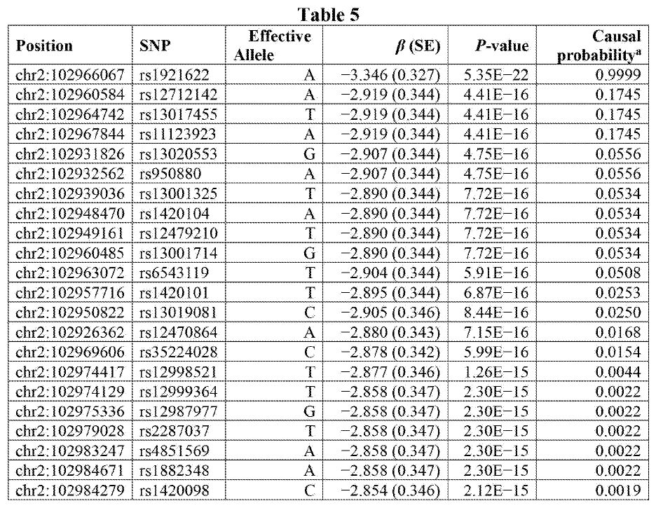

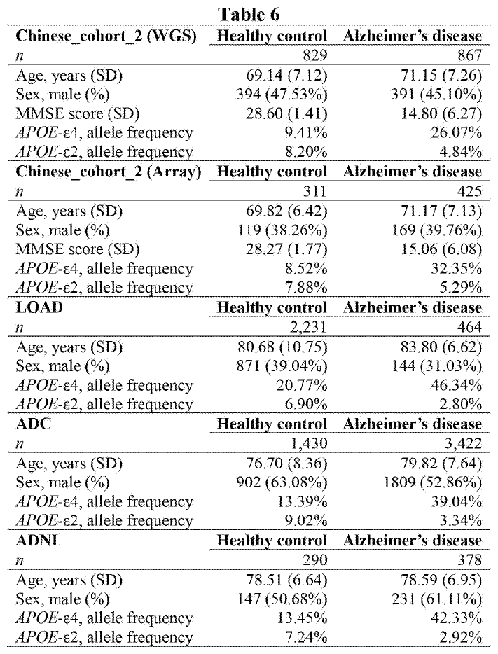

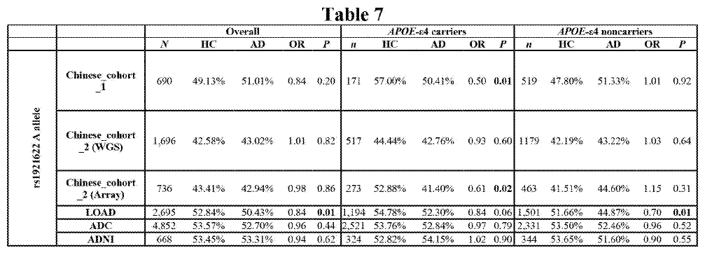

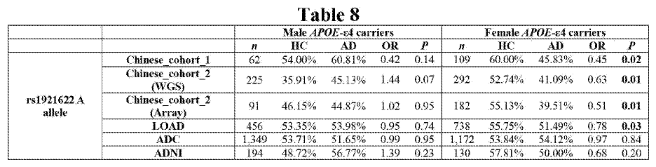

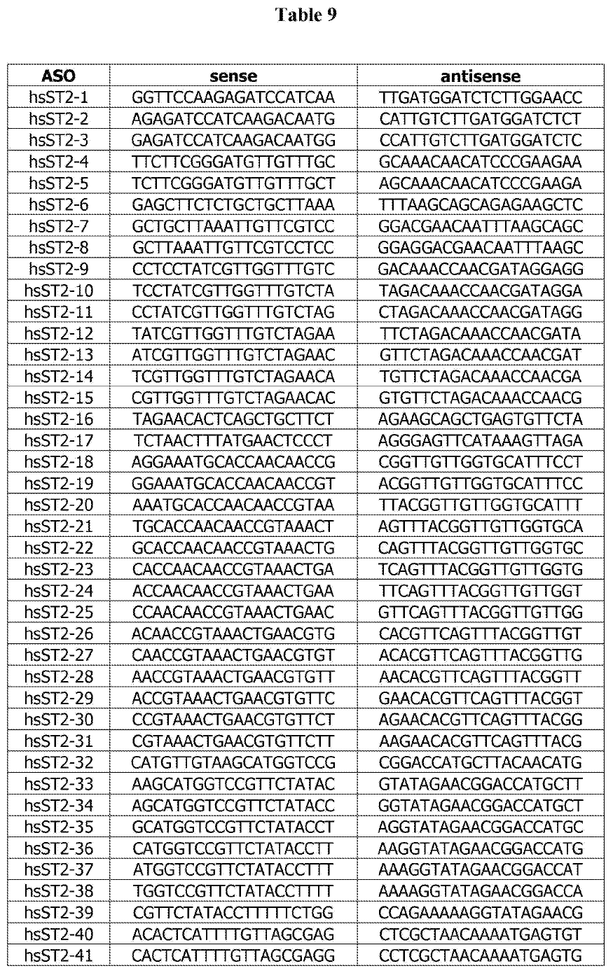

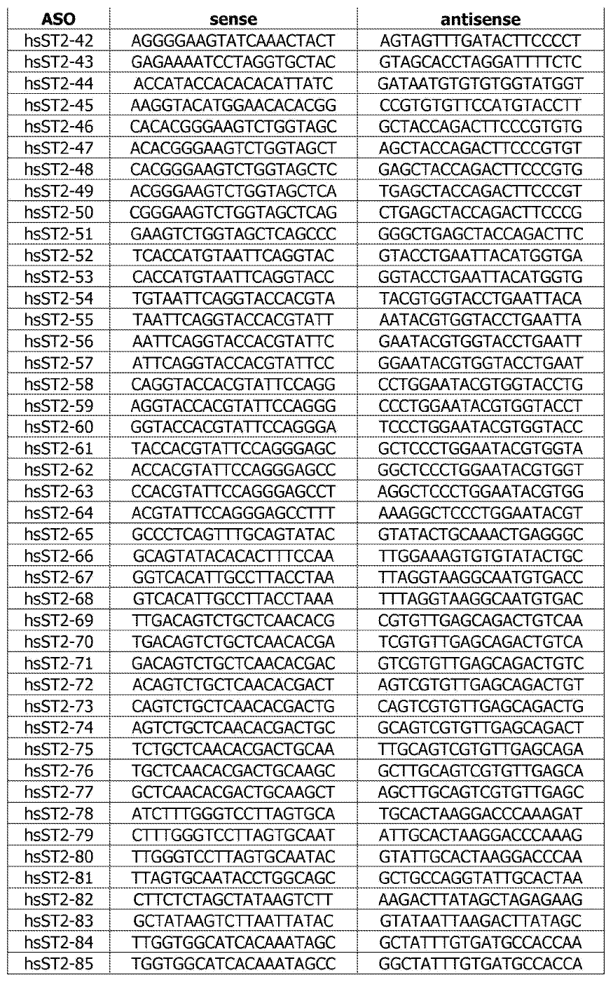

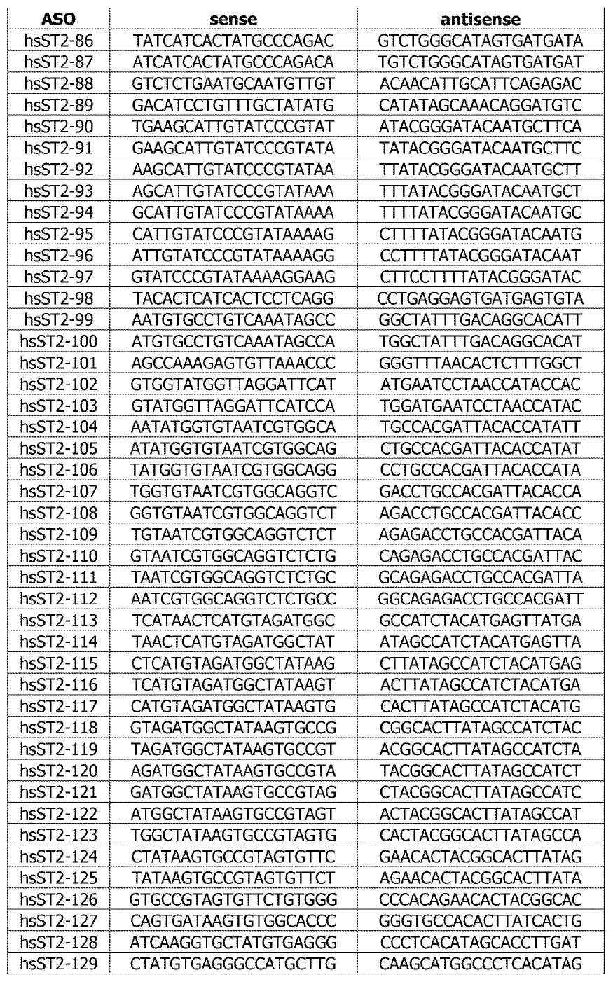

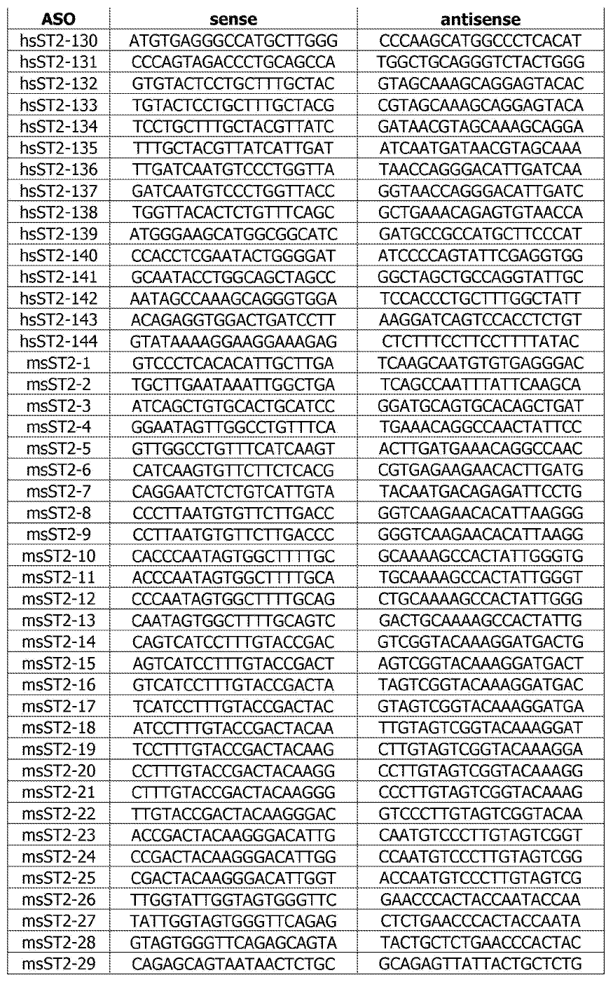

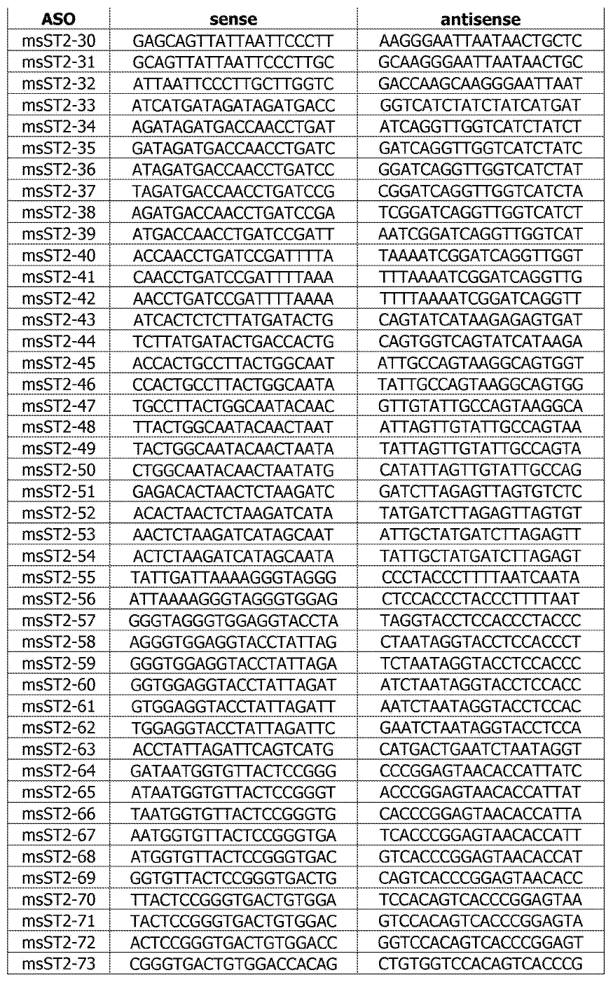

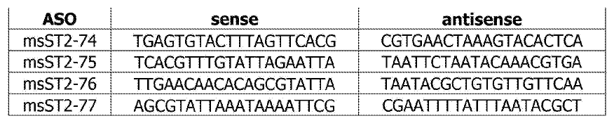

As antisense oligonucleotides could target the 3′UTR region, trigger the RNase H-mediated degradation of mRNA and eventually reduce the mRNA level of target genes (FIG. 24a), it might serve as a good translational tool to manipulate the sST2 level. We performed the systematic screening for all potential ASOs that can specific target to sST2 3′UTR region in mouse fibroblast cells NIH 3-3 and Human Umbilical Vein Endothelial Cells (HUVEC) (FIG. 24b, FIG. 25a, Table 9). We performed a second run of screening with top 13 msST2-ASOs and top 15 efficient hsST2-ASOs and examined the sST2 transcript level. Most of the ASOs showed more than 50% of the reduction of the sST2 transcript (FIG. 24c, FIG. 25b). Moreover, intravenous injection of mouse sST2 ASO reduces serum sST2 level in C57 mice (FIG. 24d) and also decrease AD load in 5XFAD mice (FIG. 24e,). This provide possible hints for the future clinical translation.

### DISCUSSION

GWAS studies suggest that most AD risk genes are enriched in microglia and that changes in their expression regulate the phagocytic functions of microglia⁶. Nonetheless, emerging studies suggest that soluble factors in the brain milieu also modulate microglial activities and disease-related pathologic changes^{20,23}. Here, we report that the expression of the endothelial gene IL1RL1, which encodes sST2 (a secreted decoy receptor for IL-33/ST2 signaling), is modulated by a genetic variant, rs1921622; this variant exerts a protective effect against AD through the modulation of plasma and CSF sST2 levels, which in turn regulate microglial phenotypes and Aβ accumulation in AD. While elevated sST2 levels associate with higher Aβ deposition in patients with AD and exacerbate Aβ plaque formation and impair microglial clearance of Aβin a mouse model of amyloidosis, we show that rs1921622 downregulates sST2 expression; this variant protects against neurodegeneration, cognitive decline, and Aβ accumulation in female APOE-ε4 carriers who tend to have a higher risk of AD and more-severe AD-related pathologic changes⁵⁹. Analysis of postmortem human brains suggests that the protective effect of rs1921622 is mediated by the regulation of microglia-Aβ plaque interactions. Thus, our results collectively show that sST2, a soluble protein secreted by endothelial cells, regulates the activities of microglia and that alterations in sST2 levels in the brain milieu impair the AD-clearance capacity of microglia, thus modulating the risk and pathologic changes of Aβassociated with APOE-ε4.

As sST2 only comprises the extracellular domain of ST2L and is independently transcribed²⁶, it is an effective decoy receptor for IL-33/ST2 signaling. Given that IL-33/ST2 signaling has essential regulatory roles for microglial activities involved in tissue repair, Aβ clearance, and synapse engulfment^{28,29,60,61} an increased brain sST2 level likely impacts microglial functions and AD-related pathologic changes by blocking the binding of IL-33 to ST2L on microglia. In AD, the accumulation of Aβtriggers microglia to migrate towards AD plaques, extend processes to form barriers surrounding then, and initiate phagocytic clearance⁶². As AD progresses, these microglial functions become impaired, which results in the accumulation of Aβ plaques⁶³. In this study, sST2 injection in a mouse model of amyloidosis perturbed the interaction between microglia and AD as well as the subsequent phagocytosis of Aβ(FIG. 4). This finding is consistent with our previous observation that in these mice, IL-33 administration activates IL-33/ST2 signaling and initiates microglial chemotaxis towards Aβ plaques, subsequently enhancing AD phagocytosis^{28,29}. Further corroborating the pathological role of sST2 in microglia in AD, among patients with AD carrying APOE-ε4, those carrying rs1921622 (who have lower sST2 levels in the brain milieu) exhibited enhanced microglia-Aβ plaque interactions and a smaller Aβ plaque area than noncarriers. Thus, perturbation of endogenous IL-33/ST2 signaling by sST2 may lead to impaired microglial chemotaxis, barrier formation, and AD uptake-all of which subsequently contribute to AD pathogenesis.

What are the regulatory mechanisms of sST2? Our analyses in the Chinese population and populations of European descent corroborate with previous studies that sST2 levels are associated with age, sex, and genetic variants^{42,44}, and further reveal that genetic components play a dominant role in the regulation of sST2 levels, accounting for 54.86% of the variance of sST2 levels. In particular, our GWAS and fine-mapping analyses identified rs1921622 as a putative causal variant associated with sST2 (causal probability=99.99%); our ChIP assay and CRISPR/Cas9 editing experiment verified that the rs1921622-containing region is an enhancer element that regulates sST2 expression in endothelial cells. These results collectively suggest that rs1921622 is a key genetic modulator of sST2. As such, future investigations on the epigenetic events at the rs1921622 locus may help elucidate the regulatory mechanisms of sST2. Indeed, recent studies suggest that activation of TNFα-mediated NF-κB signaling can induce the expression and release of sST2 from endothelial cells^{64,65} and that inhibition of NF-κB signaling abolishes sST2 production⁶⁶. Given that NF-κB is an essential transcription factor that regulates gene expression in endothelial cells⁶⁷, it would be of interest to investigate whether NF-κB is a candidate transcription factor involved in the rs1921622-mediated regulation of sST2 expression in endothelial cells.

In the present study, single-nucleus transcriptomic profiling of postmortem human brains shows that sST2 is mainly expressed in brain endothelial cells (FIG. 11). The dysregulation of sST2 expression in the brain exacerbates AD-associated pathologic changes, specifically impairing the phagocytic capacity of microglia. Thus, our results demonstrate a novel pathogenic role of the brain vasculature in AD that mediates the activities of microglia in the brain via a secreted soluble factor, sST2. Indeed, apart from AD and tau-related pathologic changes, neurovascular dysfunction occurs early in AD and is implicated in its pathogenesis⁶⁸. Brain transcriptomic profiling has revealed numerous dysregulated genes in endothelial cells in AD that are associated with angiogenesis and antigen presentation⁵⁰. Moreover, APOE-ε4 was recently shown to exacerbate blood-brain barrier breakdown, which results in the leakage of blood-derived proteins such as thrombin and plasmin^{69,70}, leading to synapse loss^{71,72}. While the exact pathological functions of the brain vasculature that cause AD remain unclear, in the peripheral system, vasculature-secreted soluble cytokines and chemokines (e.g., CXCL 1) commonly regulate the activation and migration of immune cells, thereby mediating the immune response in tissues⁷³. Similarly, emerging studies suggest that the brain vasculature is not only responsible for supplying nutrients and clearing metabolites but also serves as a critical source of soluble inflammatory proteins such as IL-1β, IL-6, IL-8, TNFα, TGFβ, and MCP-1^{74,75}. Therefore, besides sST2, other soluble factor-based crosstalk between the vasculature and other cell types likely occurs in the brain. Accordingly, identifying those components and mediators of such crosstalk may expand our understanding of the roles of the brain vasculature and provide insights into novel pathological mechanisms of AD.

Our genetic analyses demonstrated that rs1921622 exerts protective effects against AD in APOE-ε4 carriers, suggesting a potential interaction between IL-33/ST2 signaling and ApoE. ApoE, a lipoprotein and major constituent of Aβ plaques, has various functions including cholesterol transport, lipid metabolism, and Aβ clearance^{6,76}. In the brain, ApoE is mainly produced by astrocytes, and its expression is upregulated in microglia under neuropathological conditions including AD^{12,77}. Single-cell RNA sequencing of amyloidosis mouse models has revealed that a microglial subpopulation transitions from a homeostatic state to an activated state termed “disease-associated microglia” or “activated response microglia”^{6,57}. This activated state is characterized by increased expression of microglial activation genes (including APOE, AXL, TREM2, and CD74)^{12,13,57,58} that are associated with pattern recognition, lipid metabolism, and lysosomal pathways and are crucial regulators of phagocytic processes including detection, engulfment, and degradation⁷⁸⁻⁸¹. In contrast, perturbation of the functions of APOE and TREM2 in the brain abolishes the induction of this microglial activation state and locks microglia in a homeostatic state, resulting in lower AD phagocytic capacity^{6,57}. Thus, ApoE-mediated microglial activation may have a protective role against AD and be required for AD clearance and brain homeostasis. In the present study, snRNA-seq analysis of human postmortem brains revealed that in patients with AD carrying APOE-ε4, the presence of rs1921622 modulates the transition of microglia from a homeostatic state to an activated state characterized by increased expression of the aforementioned microglial activation genes (FIG. 21). Therefore, IL-33/ST2 signaling and ApoE might converge to regulate the expressions of these specific genes in microglia and thereby modulate the activation state and AD-clearance capacity of microglia. Interestingly, in the periphery, IL-33 administration ameliorates the formation of macrophage foam cells (lipid-laden macrophages causing atherosclerosis) and the development of atherosclerotic plaques in the ApoE^{-/-} model of atherosclerosis⁸². Therefore, it would be of interest to determine whether the modulation of IL-33/ST2 signaling reduces the detrimental effects of APOE-ε4 on AD accumulation through the regulation of lipid metabolism in AD. Indeed, ABCA1 (ATP-binding cassette transporter A1) and ABCG1 (ATP-binding cassette transporter G1), which are responsible for cholesterol efflux⁸³, are among the microglial genes whose expression is upregulated by rs1921622. Moreover, these 2 genes play essential roles in ApoE lipidation^{84,85}, which is involved in Aβ degradation⁸⁶ but is impaired in APOE-ε4 carriers^{87,88}. While these 2 genes may be the key components shared by both ApoE and IL-33/ST2 mediated signaling in AD, how they are involved in the crosstalk between these two signaling pathways await further studies.

Our findings collectively suggest that sST2 is a promising therapeutic target for AD. First, as sST2 is mainly expressed by endothelial cells, this enables cell-type-specific manipulation of sST2 expression and that manipulation may not need to cross the blood-brain barrier. Second, sST2 levels are elevated in patients with mild cognitive impairment or early-stage AD^{28,36}, suggesting the potential applicability of sST2 in early intervention strategies. Third, the deletion of the rs1921622 locus, which we showed can be done with high efficacy in a human brain endothelial cell line, could be a feasible method to specifically silence sST2 expression and secretion without disrupting the activities of ST2L; this is because the epigenetic and transcriptional controls of sST2 are distinct from those of ST2L⁸⁹, and rs1921622 only modulates the expression of sST2 but not ST2L. Fourth, as we showed that rs1921622 is a common AD-associated variant, manipulations of sST2 targeting this genetic variant could be developed for specific subgroups of patients who have high sST2 levels (e.g., female patients who carry APOE-ε4 but not the rs1921622 A allele, accounting for 6.2%-12.2% of patients with AD), enabling patient stratification and precision medicine. In addition, as sST2 is also a well-known biomarker of CVDs^{35,37} with potential pathogenic roles in atherosclerosis and sepsis^{82,90}, such genome-based manipulations targeting sST2 might also be beneficial for the treatment of such peripheral diseases.

Nonetheless, more remains to be illustrated regarding the functions and regulation of sST2 in AD. First, although we demonstrated that the increased sST2 level in the brain contributes to AD pathogenesis, it remains unknown whether plasma sST2, which constitutes the major pool of sST2 produced by peripheral endothelial cells⁹¹, plays pathological roles in AD. The positive correlation between plasma and CSF sST2 levels suggests that sST2 may be blood-brain barrier permeable. Therefore, further investigation is needed to confirm whether peripheral sST2 can permeate the brain parenchyma and contribute to disease-related pathologic changes. Indeed, recent studies have shown that several angiotensin receptor blockers, such as valsartan, can reduce peripheral sST2 levels in patients with heart failure⁹². It is of interest to examine whether these drugs can also regulate peripheral sST2 levels in patients with AD and ameliorate the disease-related pathologic changes. Second, our association analyses in multiple cohorts consistently demonstrated that rs1921622 protects against AD in APOE-ε4 carriers, specifically females. While the variant modulates sST2 expression in both males and females, the reason for this sex-specific protective effect in AD remains unclear. Of note, sex hormones including estrogen and testosterone have distinct regulatory effects on IL-33/ST2 signaling^{93,94}. Therefore, it will be of interest to examine whether such signaling has different activities and functions in males and females in AD. Lastly, as IL-33, ST2L, and sST2 all contribute to IL-33/ST2 signaling, besides increased sST2 levels, the dysregulation of IL-33 and/or ST2L may also contribute to AD. Recent studies show that genetic variants in the IL33 gene are associated with AD risk⁹⁵ and that brain IL-33 transcript and protein levels are lower in AD than physiological conditions^{36,95}. Therefore, future integrative studies of sST2, IL-33, and ST2L in AD at both the genomic and gene levels may further clarify how impaired IL-33/ST2 signaling contributes to AD pathogenesis.

In summary, we uncovered an alternative pathogenic mechanism of AD that involves microglial dysfunctions mediated by a soluble protein, sST2, in the brain. Dysregulation of endothelial cell-secreted sST2 leads to increased plasma and CSF levels of sST2 and impairs Aβ clearance by microglia, resulting in exacerbated AD accumulation in AD. Furthermore, we found that the AD-protective genetic variant rs1921622, which downregulates sST2 expression, attenuates the APOE-ε4-related risk and pathologic changes of AD through the regulation of microglial signaling. Concordantly, decreasing sST2 expression and protein levels by 3′-UTR-targeting ASOs or genome editing ameliorated AD-related pathological changes. Thus, a better understanding of how the circulating levels of sST2—a novel biomarker and potential drug target for AD—are genetically regulated can aid the design of intervention strategies and clinical trials.

### Methods and Materials

### Participant Recruitment for the Chinese_Cohort_1

A total of 690 Hong Kong Chinese participants ≥60 years old, including 345 patients with AD and 345 HCs, who visited the Specialist Outpatient Department of the Prince of Wales Hospital of the Chinese University of Hong Kong from April 2013 to February 2018 were recruited. A clinical diagnosis of Aβwas established on the basis of the American Psychiatric Association's Diagnostic and Statistical Manual of Mental Disorders, Fifth Edition (DSM-5)⁶⁶. All participants underwent medical history assessment, clinical assessment, cognitive and functional assessment using the Montreal Cognitive Assessment (MoCA) test, and neuroimaging assessment by MRI^{97,98}. Participants with any significant neurologic disease besides AD or psychiatric disorder were excluded. Age, sex, years of education, medical history, and history of CVDs (i.e.. heart disease, hypertension, diabetes mellitus, and hyperlipidemia) were recorded. DNA and plasma samples were prepared from whole-blood samples and stored at −80° C. until use. T1-weighted MRI was used to retrieve brain imaging data from 192 participants (n=77 patients with AD, n=115 HCs) from Prince of Wales Hospital. Raw imaging files were deidentified and processed by AccuBrain® IV1.2 (BrainNow Medical Technology Ltd, Hong Kong, China) for analysis of gray matter volumes. A subcohort consisting of only participants without a history of CVDs was also selected from the Chinese_cohort_1 (“non-CVD cohort”; n=86 patients with AD, n=97 HCs).

This study was approved by the Prince of Wales Hospital, the Chinese University of Hong Kong, and the Hong Kong University of Science and Technology. All participants provided written informed consent for both study enrollment and sample collection.

DNA and Plasma Extraction from Human Blood Samples

We collected whole-blood samples (3 mL) from participants into K3EDTA tubes (VACUETTE). We centrifuged the samples at 2,000×g for 15 min to separate the cell pellet and plasma. The plasma was collected, aliquoted, and stored at −80° C. until use. We sent the cell pellets to the Centre for PanorOmic Sciences (Genomics and Bioinformatics Cores, University of Hong Kong, Hong Kong, China) for genomic DNA extraction using the QIAsymphony DSP DNA Midi Kit (QIAGEN) on a QIAsymphony SP platform (QIAGEN). Genomic DNA was eluted with water or Elution Buffer ATE (QIAGEN) and stored at 4° C. We determined the DNA concentration by BioDrop μLITE+(BioDrop).

### UKBBN Dataset

The following samples were obtained from the MRC UKBBN (Bristol Brain Bank): CSF samples, frontal cortex sections, frozen frontal cortex tissues and genomic DNA samples (Table 3). For initial sample selection from the UKBBN dataset, subjects with other neurodegenerative diseases, vascular diseases, an intoxicated state, infection, prions, inflammatory diseases, structural brain disorders, metabolic/nutritional diseases, trauma, delirium, genetic disorders (e.g.. Down syndrome), or other systemic diseases were excluded. For CSF samples, samples with a postmortem duration ≤30 h were selected, yielding a total of 86 participants (n=75 patients with AD, n=11 HCs). In addition, snRNA-seq data from frozen frontal cortical samples from the UKBBN (n=12 patients with AD, n=9 HCs) were obtained from our previously published dataset⁵⁰.

### Other Cohorts and Data for Association Studies

The following data were obtained for replication studies: (i) genomic, demographic, and clinical data from the Chinese_cohort_2, wherein the participants were recruited as previously described⁵²; (ii) genomic, demographic, and clinical data from the LOAD Family Study⁵³; (iii) genomic, demographic, and clinical data from the NIA ADC cohort^{54,55}; (iv) genomic, demographic, clinical and brain imaging data from the ADNI cohort; (v) genomic, demographic, and transcriptomic data from the GTEx dataset^{48,49}; (vi) plasma biomarker, CSF biomarker, demographic data, and clinical data from the Stanford Alzheimer's Disease Research Center (ADRC) cohort⁴⁰; (vii) plasma biomarker and demographic data of the INTERVAL and LonGenity cohorts⁴² retrieved from the online database (https://twc-stanford.shinyapps.io/aging_plasma_proteome/); (viii) CSF biomarker and demographic data from a Japanese cohort⁴³; and (ix) genomic, demographic, clinical and brain imaging data from the AIBL cohort⁵⁶.

### Measurement of Protein Levels in Human Samples and Cell Lines

The plasma level of sST2 in 613 participants from the Chinese_cohort_1 (n=277 patients with AD, n=336 HCs), CSF level of sST2 in 86 participants from the UKBBN cohort (n=75 patients with AD, n=11 HCs), and level of sST2 secreted by hCMEC/D3 cells were measured using the Human ST2/IL-33 R Quantikine ELISA Kit (DST200; R&D Systems). The plasma levels of NfL (n=135 patients with AD, n=116 HCs) and P-tau181 (n=145 patients with AD, n=126 HCs) in participants from the Chinese_cohort_1 were measured by Quanterix Accelerator Lab (Boston, MA, USA) using the Quanterix NF-light SIMOA Assay Advantage Kit (103186) and P-Tau 181 Advantage V2 Kit (103714), respectively.

### Whole-Genome Sequencing and SNP Array for Genotyping

DNA samples from 427 participants from the Chinese_cohort_1 (n=233 patients with AD, n=194 HCs) were submitted to Novogene (Shenzhen, China) for library construction and whole-genome sequencing. The samples were sequenced on the Illumina HiSeq X platform (average depth: 5×), and individual genotypes were analyzed using the GotCloud pipeline⁴⁶. An SNP array was used to genotype 263 genomic DNA samples from the Chinese_cohort_1 (n=112 patients with AD, n=151 HCs) and 113 genomic DNA samples from the UKBBN cohort (n=102 patients with AD, n=11 HCs), for the genotyping of chr2:102966067 (GRCh37/hg19), APOE-42, and APOE-ε4 using TaqMan Assays (rs1921622, C_1226146_10, Cat No. 4351376; rs7412, C_904973_10, Cat No. 4351376; and rs429358, C_3084793_20, Cat No. 4351376, respectively; Thermo Fisher Scientific). We performed real-time quantitative PCR using the 7500 Fast and QuantStudio 7 Flex Real-Time PCR System (Applied Biosystems). We stored the results in EDS files and input them into TaqMan Genotyper Software (Applied Biosystems) for the joint genotyping of SNPs.

### Immunohistochemical Staining of Postmortem Human Brain Sections

Formalin-fixed, paraffin-embedded, postmortem, frontal cortex sections from 78 patients with AD were obtained from the UKBBN cohort. The sections were first deparaffinized and rehydrated with xylene and graded ethanol solutions. To stain Aβ, the sections were first treated with formic acid at room temperature for 5 min. Endogenous peroxidase activity was quenched with a 3% hydrogen peroxide solution. The sections were then incubated with a mouse anti-human Aβ antibody (1:500, clone NAB228, SC-32277, Santa Cruz Biotechnology) overnight at 4° C. After washing, the sections were incubated with HRP-labeled anti-mouse IgG (QD440-XAKE, RTU, BioGenex), and signals were developed with 3,3′-diaminobenzidine (DAB) substrate (QD440-XAKE, BioGenex). To co-stain microglia and AD protein, double immunohistochemical staining was performed; after deparaffinization and rehydration, the sections were treated with sodium citrate buffer (10 mM sodium citrate, pH 6.0) for 25 min and blocked, after which endogenous peroxidase activity was quenched by 3% hydrogen peroxide solution. The sections were then incubated with the mouse anti-human Aβ antibody (SC-32277) and rabbit anti-human Iba-1 antibody (1:100, 019-19741, polyclonal, FUJIFILM Wako Pure Chemical Corporation) overnight at 4° C. After washing, the sections were incubated with HRP-labeled anti-mouse Ig and AP-labeled anti-rabbit Ig (HK597-50K, Double Staining kit, BioGenex) followed by substrate development with DAB (QD440-XAKE, BioGenex) and Fast Red Substrate (HK182-5KE, BioGenex). The sections were then counterstained with Mayer's hematoxylin (HK100-9K, BioGenex) and mounted with coverslips. Buffer used for washing was Tris Buffer Saline (TBS) with 0.01% Triton X-100 and primary antibodies were diluted in TBS. Images were taken with a ZEISS Axio Scan.Z1 scanner and processed with ZEN microscope software v3.2 (ZEISS).

To quantify Aβ plaques, we took 10 random images of each section. After background subtraction and threshold adjustment, we analyzed the Aβ plaques using the Analyze Particles function in Fiji-ImageJ (v1.53c). We determined the total AD area, number of Aβ plaques, and median plaque size for each section. We calculated Aβ plaque load (% area) by dividing the total Aβ area by the total image area (10 mm). To quantify microglia-Aβ co-staining, we selected 20 random images of each section and processed them with the Colour Deconvolution function to separate the data into 3 color channels (i.e.. DAB, Fast Red, and hematoxylin). After adjusting the threshold, we selected Aβ plaques and microglia using the Create Selection function, then analyzed them using the Analyze function. We determined the total AD area and AD area co-localized with Iba-1 staining. We calculated the Aβ plaque area co-localized with microglia (% total Aβ) by dividing the Aβ area co-localized with Iba-1 staining by the total AD area. Two independent researchers performed section staining, image acquisition, and image quantification; they also randomly selected and quantified images in a blinded manner.

### Association Analysis and Data Visualization for the GWAS

The association analysis between SNPs and plasma sST2 level at the genome-wide level in the Chinese_cohort_1 was performed with PLINK software (v1.9)⁹⁹, adjusting for age, sex, AD diagnosis, and the top 5 principal components as covariates, with the following parameters: -keep-allele-order, -linear, -ci 0.95, -hwe 0.00001, and -maf 0.05. To visualize the data, a Manhattan plot and quantile-quantile plot were generated using the manhattan( ) function and qq( ) function of the R qqman package (vW.1.4), respectively. Regional plots for the IL1RL1 locus were generated using LocusZoom. Fine-mapping analysis of the effects of the IL1RL1 locus on the plasma sST2 level was performed using CAVIAR software (v2.2)⁴⁷ with association test results and pairwise linkage disequilibrium information generated from PLINK software (v1.9) with the following parameters: -hwe 0.00001, -maf0.05, -r, -matrix, -chr 2, -from-bp 102000000, and -to-bp 104000000. The fine-mapped regional plot was generated using the plot_1ly( ) function of the R plotly package (v4.9.1). Linkage disequilibrium and haplotype structures were plotted using Haploview (v4.2). To identify all independent sST2-associated variants (r²<0.2), variants with P<1E-5 according to the sST2 GWAS were subjected to analysis by PLINK software (v1.9) (parameters: -hwe 0.00001, -maf0.05, -clump-p1 0.00001, -clump-r2 0.2, -chr 2, and -clump-kb 2000) yielding 29 independent sST2-associated variants. The calc.relimp( ) function of the R relaimpo package (v2.2-3)^{100,101} was used to quantify the contributions of genetic factors (i.e.. the 29 independent sST2-associated variants) and nongenetic factors (i.e., age and sex) to sST2 level variance.

### Association Analysis of Rs1921622 in Transcriptome Datasets at Tissue and Single-Cell Levels

Human tissue sST2 and ST2L transcript levels as well as rs1921622 genotype data from the GTEx dataset^{48,49} were used for the genotype-expression association test, adjusting for age, sex, RNA integrity (i.e.. RNA integrity number), and population structure (i.e., the top 4 principal components). Rank-based normalization of transcript levels was performed using the R rntransform( ) function of the GenABEL package (v1.8).

The transcript levels of sST2 and ST2L in the human frontal cortex at the single-cell level were obtained by realigning the FASTQ files of our previously published snRNA-seq dataset⁵⁰ using a modified reference genome. Specifically, the IL1RL1 region (chr2: 102,311,563-102,352,037) in the GTF file of the original GRCh38/hg38 pre-mRNA reference genome was separated into 3 parts: the sST2-specific region (chr2: 102,343,416-102,346,100), ST2L-specific region (chr2: 102,311,563-102,337,147 and 102,346,101-102,352,037), and overlapping region (chr2: 102,337,148-102,343,415). A modified reference genome was generated by Cell Ranger (v3.0.1) using the new GTF file and original FASTA file. In the subsequent quality control step, the quantification of gene levels and cell-type identification were performed as previously described⁵⁰. For the association analysis between genotype and candidate gene expression in each cell cluster, linear regression analysis was performed, adjusting for age, sex, AD diagnosis, and postmortem duration. The level of significance was set at an FDR-adjusted P<0.05. GO analysis of associated genes was performed using DAVID Bioinformatics Resources^{102,103}.

### Analysis of the Association Between Rs1921622 and Alzheimer's Disease Risk

A meta-analysis was performed to examine the effects of rs1921622 genotype on AD risk. Specifically, the effect sizes (i.e., log odds ratios) and standard errors (SEs) for APOE-ε4 carriers and non-carriers from 6 AD datasets (i.e., the Chinese_cohort_1 dataset, the WGS and array datasets of Chinese_cohort_2, and LOAD, ADC, and ADNI datasets) were determined using logistic regression with age, sex, and the top 5 principal components as covariates. The results were summarized and processed by METASOFT (v2.0.0)¹⁰⁴ to estimate the joint risk effects and significance levels under Han and Eskin's random effects model (RE2). The results were then input to ForestPMPlot (v1.0.2) to generate forest plots for data visualization.

### In Vivo Experiments in Mice

We housed all mice in the HKUST Animal and Plant Care Facility. All animal experiments were approved by the HKUST Animal Ethics Committee and conducted in accordance with the Guidelines of the Animal Care Facility of HKUST. We housed 4 mice of the same sex per cage at 22° C. and at a relative humidity of 60%, with a 12-h light/dark cycle as well as food and water ad libitum. Wild-type (WT) C57BL6J mice were obtained from the Jackson Laboratory. The 5XFAD mice were generated as previously described by overexpressing the K670N/M671L (Swedish), I716V (Florida), and V717I (London) mutations in human APP as well as the M146L and L286V mutations in human PSEN1¹⁰⁰. We confirmed genotypes by PCR analysis of tail or ear biopsy specimens. sST2 3′UTR deletion mice were generated by GemPharmatech Co., Ltd. The sST2 3′UTR deletion mice were generated by CRISPR/Cas9-mediated deletion. The sequence of gRNAs were 5′-GTCCCTTGTAGTCGGTACAA-3′ and 5′-GACACTCTACTTGTACCTAG-3′. We confirmed genotypes by PCR analysis of tail or ear biopsy specimens. We performed all in vivo experiments on age-matched groups and randomly assigned the mice to the experimental conditions. We chose our sample sizes primarily based on our experience with similar types of experiments. We conducted all animal experiments during the light phase. Murine recombinant sST2-Fc (1004-MR-050; R&D Systems) was delivered into 5XFAD mice (B6.Cg-Tg(APPSwFlLon,PSEN1*M146L*L286V)6799Vas/Mmjax) via mini-osmotic pumps (model 1004; Alzet) at 0.11 pIJh. Specifically, the pumps were implanted intracerebroventricularly above the right hemisphere and loaded with murine recombinant sST2-Fc protein (240 ng per pump; 10 μg/mL) or human IgG (as a control) in artificial cerebrospinal fluid (119 mM NaCl, 2.5 mM KCl, 2.5 mM CaCl₂·2H₂O, 1 mM NaH₂PO₄·2H₂O, 1.3 mM MgCl₂·6H₂O, 26.2 mM NaHCO₃, and 11 mM D-glucose). After 28 days of administration, the mice were anesthetized with isoflurane and transcardially perfused with phosphate-buffered saline (PBS), and their brains were collected. 40 mg/kg of mouse sST2 ASO was intravenously injected into C57 mice and 5XFAD mice. After 5 days of administration, the mice were anesthetized with isoflurane and perfused with PBS, and the serum and brains were collected.

### Immunohistochemical Staining of Mouse Brains

The left hemispheres of the mouse brains were fixed in 4% paraformaldehyde at 4° C. for 24 h, transferred to 30% sucrose, and stored at 4° C. until sectioning. The brains were cut coronally into 50-μm sections with a vibrating blade microtome (VT1000S, Leica) and stored in cryoprotectant solution (30% glycerol, 30% ethylene glycol, and PBS) at −20° C. until use. For immunohistochemistry, the sections were rinsed with PBST (i.e.. 0.1% Triton X-100 in PBS) and then treated with formic acid at room temperature for 5 min for antigen retrieval followed by 3% hydrogen peroxide solution for 10 min to quench endogenous peroxidase activity. The sections were blocked in 5% horse serum in PBST for 2 h and then labeled with 4G8 antibody (1:1000, 800703, BioLegend) in blocking buffer overnight at 4° C. The next day, the sections were incubated with biotin-conjugated anti-mouse secondary antibodies (1:1000, BA2000, Vector Laboratories) followed by an avidin-biotin-HRP complex (PK-6100, Vector Laboratories), and signals were developed with DAB (SK-4100, Vector Laboratories). Imaging was performed using a Leica DM6000 B compound microscope. The Aβ plaque areas in cortical sections were analyzed using the Analyze Particles function of Fiji-ImageJ (v1.53c). Specifically, 4 brain sections per mouse in cortical regions (˜200-300 μm apart) were analyzed, and the average percentage of the cortical area occupied by Aβ plaques was calculated.

For immunofluorescence analysis, we washed sections and incubated them in 1 μM X-34 for 10 min. We then washed them in X-34 buffer (40% EtOH in PBS), and then in PBS. We then blocked sections for 2 h in blocking buffer (4% horse serum, 1% bovine serum albumin [BSA], and 0.3% Triton X-100 in PBS). Primary antibodies used in experiments include mouse anti-AD antibody (1:1000, clone 4G8, 800703, BioLegend), rabbit anti-Iba-1 (1:1000, 019-19741, Wako), and rat anti-Ki67 (1:200, clone SolA15, 14-5698-80, eBioscience); we diluted these in blocking buffer and incubated sections overnight at 4° C. Sections were subsequently incubated with fluorophore conjugated secondary antibodies against mouse, rabbit, and rat Ig (Alexa Fluor 488, 568, and 647; 1:1000, Life Technologies) in blocking buffer for 2 h at RT; extensively washed in PBST; stained with SYTOX Green (1:300000, S7020, Life Technologies) or DAPI (1:5000, D3571, Life Technologies); and mounted using FluorSave™ Reagent (345789, Millipore).

We performed imaging using a Leica TCS SP8 confocal microscope with a Leica 40× oil immersion objective. We took 5 images from each mouse cortex with a step size of 1 μm for a total of 40 μm, then merged them into a single image with maximum intensity Z-projection. We identified 3 different plaque morphologies using anti-AD immunolabeling (i.e.. 4G8) and X-34 staining: (i) filamentous plaques characterized by filamentous X-34 and 4G8 labeling with no plaque core; (ii) compact plaques characterized by 4G8 amyloid fibrils projecting radially outward with an X-34-labeled core; and (iii) inert plaques characterized by an X-34-labeled core with no 4G8 labeling. We used a custom macro to segment individual Aβ plaques and identified the type of each plaque manually. For plaque-associated microglia, we counted the numbers of microglia surrounding small (i.e., radius ≤8 μm) and large plaques (i.e., radius>8 μm) manually, as defined by DAPI⁺ nucleus staining within the barrier surrounding the plaques and processes in contact with the plaques. We defined the Ki67⁺ microglia as microglia with Ki67 signals within the nucleus. To quantify microglial coverage of Aβ plaques, we selected only compact plaques. We used 10 optical slices 0.5 μm apart through the center of the plaque for analysis. We processed all images with a customized macro in Fiji-ImageJ (v1.53c). On each slice, after adjusting the threshold, we determined the perimeters of the plaque using the Analyse Particles and Area to Line functions. We also determined plaque perimeters and the arcs of plaque perimeters co-localized with microglial staining. We calculated the proportion of the plaque perimeter covered by microglia by summing the arcs of the plaque perimeter across 3-dimensional (3D) stacks in close contact (within 1 μm) with Iba-1-immunolabeled cells (˜25 plaques per group). We conducted the 3D reconstruction of microglia-plaque interaction using Imaris v9.7.2 (Oxford Instruments).

### Assessment of Microglial Aβ Phagocytic Capacity

Microglial AD phagocytic capacity was examined as previously described²⁹. Briefly, 4-month-old 5XFAD or wild-type mice were intraperitoneally injected with methoxy-X04 (10 mg/kg) to label AD. The mice were anesthetized with isoflurane 3 h after methoxy-X04 injection, and the left ventricle was perfused with ice-cold PBS. Their forebrains were isolated, minced, and incubated at 37° C. for 30 min in 5 U/mL papain (LS003126) and 35 U/mL DNase I (LS002140; Worthington Biochemical) for enzymatic digestion. After incubation, myelin debris was depleted by 30% isotonic Percoll (P1644; Sigma-Aldrich) gradient centrifugation, and mononuclear cell suspensions were obtained in DMEM/F12 medium with ice-cold 10% heat-inactivated FBS. Unstained controls were prepared from mixtures of different sample cell suspensions for cell population identification. To label microglia, an Alexa Fluor 488-conjugated mouse CD11b antibody (1:200; 53-0112-82; eBioscience) was used to stain the cell suspensions for 45 min at 4° C. The resultant labeled cell suspensions were analyzed using a BD Influx cell sorter flow cytometer. The recorded scatterplot data for the microglial cell population were analyzed using FlowJo software v10.5.0 (TreeStar).

### CRISPR/Cas9-Mediated Genome Deletion of the Rs1921622-Harboring Region in a Cell Culture System

The human cerebral microvascular endothelial cell line (hCMEC/D3) was purchased from Cedarlane and cultured as previously described 11. Briefly, we coated a tissue culture plate with 100 μg/mL type I collagen (Millipore) at 37° C. in 5% CO₂ for 1 h. We subsequently washed the plate with Dulbecco's phosphate-buffered saline (DPBS) and replaced it with a complete culture medium (Endothelial Cell Growth Medium-2 [EGM-2][Lonza] supplemented with 5% FBS [HyClone], 1% chemically defined lipid concentrate [Gibco], 10 mM HEPES [Gibco], 5 μg/mL ascorbic acid [Sigma], 1.4 μM hydrocortisone [Sigma], 1 ng/mL bFGF [PeproTech], 10 U/mL penicillin, and 10 μg/mL streptomycin [Gibco]). Cultured cells were dissociated with 0.05% trypsin for 5 min, replated at 25,000 cells per cm², and returned to culture at 37° C. in a 5% CO₂ incubator. Three to four days after seeding, the cells reached confluence and could be passaged. We used cells at passages 27-35 for our experiments.

For the ChIP-quantitative PCR (ChIP-qPCR) experiment, we fully changed the medium of the hCMEC/D3 cells 2 h before treatment. We then treated cultured cells with recombinant human IL-33 (BioLegend) or DPBS as a vehicle control for 24 h.

To evaluate the efficiency of the single guide RNA (sgRNA) editing in endothelial cells, we transfected 5×10⁵ hCMEC/D3 cells with a single CRISPR construct by nucleofection using the Human Umbilical Vein Endothelial Cell Nucleofector Kit (Lonza) with a Nucleofector 2b device (Lonza). One day after transfection, we changed the cultured medium to a complete culture medium with 1 μg/mL puromycin (Thermo Fisher Scientific). After 3 days of puromycin selection, we extracted genomic DNA using QuickExtract™ DNA Extraction Solution (Lucigen) followed by a T7EI (NEB) editing efficiency test. All 4 sgRNAs exhibited high editing efficiency (data not shown).

To delete the region harboring rs1921622 in hCMEC/D3 cells, a dual-guide, RNA-mediated knockout approach was utilized. The cells were genotyped from 300 bp upstream and downstream of rs1921622 by Sanger sequencing. Screening of potential Streptococcus pyogenes Cas9 (SpCas9)-guided RNAs was performed using the CRISPR design tool (crispr.nmit.edu) 100 bp upstream and downstream of rs1921622. The following sgRNAs were utilized: sgRNA-1, 5′-TTATGGACAGAATTAAGAAG-3′ (SEQ ID NO:1); sgRNA-2, 5′-CTGTCCATAAGATTGAAAG-3′ (SEQ ID NO:2); sgRNA-3, 5′-AATITTGTTCTGGTAGCCAT-3′ (SEQ ID NO:3); and sgRNA-4, 5′-GGTATITCAGCTAGTGCCTA-3′ (SEQ ID NO:4). The sgRNAs were subcloned into PX459v2, which contains an sgRNA cassette, human codon-optimized SpCas9, and a puromycin resistance gene.

To generate a dual-gRNA-mediated deletion cell line, hCMEC/D3 cells were transfected with plasmids containing sgRNA-1/sgRNA-4 (targeted 67-bp deletion), or sgRNA-2/sgRNA-3 (targeted 38-bp deletion), or PX459v2 as a no-sgRNA control. After 3 days of puromycin selection (1 μg/mL) starting from the day after transfection, the puromycin-resistant cells were seeded in two 24-well plates. The culture medium was changed twice a week. After 3 weeks when a single colony was observed, the wells containing only 1 colony were passaged into a 12-well plate. Each clone was genotyped, and those in which the targeted deletion may have occurred were subjected to Sanger sequencing. This protocol generated 6 control lines, 8 lines with a 38-bp deletion, and 10 lines with a 67-bp deletion.

### CRISPR/Cas9-Mediated Genomic Editing in Human Patients

Human patients who have received a diagnosis of Alzheimer's Disease and who are genotyped to possess a genetic marker indicative of high-risk for AD (e.g., female patients APOE-ε4 carriers) are administered by intravenous injection a viral vector (e.g., an adenovirus vector or adeno-associated virus vector) or lipid nanoparticle packaged of a DNA or RNA encoding CRISPR/Cas9 system or ribonucleoprotein (RNP) complex, which encodes a Streptococcus pyogenes Cas9 (SpCas9) nuclease and two sgRNAs in a dual-guided RNA-mediated genomic editing system aiming to delete a genomic sequence encompassing the rs1921622 locus, spanning a region of about 100-bp upstream and downstream from the locus. The sgRNA targeting and deletion strategy is the same as used in the culture system (see the last section). For CRISPR editing targeting the sST2 genomic region in human patients, the lipid nanoparticles carrying sgRNAs and mRNA encoding Cas9 are delivered to patients in the range of 0.01-2 mg/kg; 0.02-1.0 mg/kg; 0.05-0.5 mg/kg; or 0.1-0.3 mg/kg of patient body weight. After 4 weeks, administration of 0.1 mg/kg of nanoparticles is expected to achieve about 50% reduction of circulating level of the target protein (e.g., sST2 protein), whereas administration of 0.3 mg/kg of nanoparticles is expected to achieve above 80% reduction of target protein level.

sST2 Antisense Oligonucleotide Screening

The specificity of sST2 antisense oligonucleotides (ASOs) were defined by no same sequence and no 1 to 2 mismatches of the sequence in other transcripts in mouse genome reference (GRCm38) and human genome reference (GRCh37). All ASOs were synthesized by Integrated DNA Technologies (IDT). To examine the effect of each ASO, Mouse fibroblast NIH-3T3 cells (ATCC) and Human Umbilical Vein Endothelial Cells (ATCC) were transfected with 500 nM ASO using Lipofectamine RNAiMAX Transfection Reagent (ThermoFisher). The cells were collected 1 days after transfection. The protein amount of cell lysis was measured to normalize the level of medium sST2 level.

### Other Statistical Analyses and Data Visualization

For the remaining statistical analyses of human subjects, the significance of the associations of Aβ-associated endophenotypes with sST2 levels and rs1921622 genotype was determined by linear regression analysis. The CSF sST2 level cutoff was determined according to the level of CSF sST2 with the maximum value of Youden's index using the optimal.cutpoints( ) function and the Youden method of the OptimalCutpoints package (v1.1-4) in R¹⁰⁵. Cox regression was performed to examine the association between the onset age of dementia and the rs1921622 A allele using the coxph( ) function of the survival package (v1.3-24) in R, with sex and the top 5 principal components as covariates. The level of significance was set to P<0.05. For data visualization, the plot( ) function of R was used to generate a volcano plot, and the ggplot( ) function of the R ggplot2 package (v3.2.1) was used to generate dot plots. For the data obtained from mouse and cell culture system experiments, the significance of differences was assessed by unpaired Student's t-tests, or one- or two-way ANOVA followed by the Bonferroni post hoc test as indicated. The level of significance was set at P<0.05. All statistical plots were generated using GraphPad Prism v8.0 (GraphPad Software).

### Data Availability

The consent forms given by individual participants from the Chinese_cohort_1 stated that the research content will be kept private under the supervision of the hospital and research team. Therefore, the phenotypic, genomic, and proteomic data of individual participants will be available and shared in formal collaborations. Applications for data sharing and project collaboration will be processed and reviewed by a review panel hosted at HKUST. Researchers may contact [sklneurosci@ust.hk] for details about data sharing and project collaboration related to the present study.

### Code Availability

All requests for code used for data analyses and data visualization will be promptly reviewed by the corresponding author and the review panel hosted at HKUST to verify the request is subject to any intellectual property, confidentiality, or other licensing obligations. If there are no limitations, the corresponding author will communicate with the requester to share the code.

All patents, patent applications, and other publications, including GenBank Accession Numbers or equivalents, cited in this application are incorporated by reference in the entirety for all purposes.

### REFERENCES

1. Association, A.s. 2019 Alzheimer's disease facts and figures. Alzheimer's & Dementia 15, 321-387 (2019).
2. Jack Jr, C. R., et al. NIA-AA research framework: toward a biological definition of Alzheimer's disease. Alzheimer's & Dementia 14, 535-562 (2018).
3. Clark, I. A. & Vissel, B. Amyloid β: one of three danger-associated molecules that are secondary inducers of the proinflammatory cytokines that mediate Alzheimer's disease. British journal of pharmacology 172, 3714-3727 (2015).
4. Wang, M.-M., Miao, D., Cao, X.-P., Tan, L. & Tan, L. Innate immune activation in Alzheimer's disease. Annals of translational medicine 6(2018).
5. Lambert, J.-C.. et al. Meta-analysis of 74,046 individuals identifies 11 new susceptibility loci for Alzheimer's disease. Nature genetics 45, 1452-1458 (2013).
6. Frigerio, C. S., et al. The major risk factors for Alzheimer's disease: age, sex, and genes modulate the microglia response to Aβ plaques. Cell reports 27, 1293-1306. e1296 (2019).
7. Yeh, F. L., Wang, Y., Tom, I., Gonzalez, L. C. & Sheng, M. TREM2 binds to apolipoproteins, including APOE and CLU/APOJ, and thereby facilitates uptake of amyloid-beta by microglia. Neuron 91, 328-340 (2016).
8. Rebeck, G. W., Reiter, J. S., Strickland, D. K. & Hyman, B. T. Apolipoprotein E in sporadic Alzheimer's disease: allelic variation and receptor interactions. Neuron 11, 575-580 (1993).
9. Kok, E., et al. Apolipoprotein E-dependent accumulation of Alzheimer disease-related lesions begins in middle age. Annals of Neurology: Official Journal of the American Neurological Association and the Child Neurology Society 65, 650-657 (2009).
10. Castellano, J. M., et al. Human apoE isoforms differentially regulate brain amyloid-β peptide clearance. Science translational medicine 3, 89ra57-89ra57 (2011).
11. Stephen, T., et al. APOE genotype and sex affect microglial interactions with plaques in Alzheimer's disease mice. Acta neuropathologica communications 7, 1-11 (2019).
12. Mathys, H.. et al. Single-cell transcriptomic analysis of Alzheimer's disease. Nature 570, 332-337 (2019).
13. Zhou, Y., et al. Human and mouse single-nucleus transcriptomics reveal TREM2-dependent and TREM2-independent cellular responses in Alzheimer's disease. Nature medicine 26, 131-142 (2020).
14. Hickman, S. E., Allison, E. K. & El Khoury, J. Microglial dysfunction and defective 0-amyloid clearance pathways in aging Alzheimer's disease mice. Journal of Neuroscience 28, 8354-8360 (2008).
15. Guillot-Sestier, M.-V., et al. 1110 deficiency rebalances innate immunity to mitigate Alzheimer-like pathology. Neuron 85, 534-548 (2015).
16. SuArez-Calvet, M., et al. Early changes in CSF sTREM2 in dominantly inherited Alzheimer's disease occur after amyloid deposition and neuronal injury. Science translational medicine 8, 369ra178-369ra178 (2016).
17. Ewers, M., et al. Increased soluble TREM2 in cerebrospinal fluid is associated with reduced cognitive and clinical decline in Alzheimer's disease. Science translational medicine 11(2019).
18. Wood, H. Soluble TREM2 in CSF sheds light on microglial activation in AD. Nature Reviews Neurology 13, 65-65 (2017).
19. Zhong, L., et al. Soluble TREM2 induces inflammatory responses and enhances microglial survival. Journal of Experimental Medicine 214, 597-607 (2017).
20. Zhong, L., et al. Soluble TREM2 ameliorates pathological phenotypes by modulating microglial functions in an Alzheimer's disease model. Nature communications 10, 1-16(2019).
21. Janelidze, S., et al. CSF biomarkers of neuroinflammation and cerebrovascular dysfunction in early Alzheimer disease. Neurology 91, e867-e877 (2018).
22. Huang, C.-W., et al. Clinical significance of circulating vascular cell adhesion molecule-1 to white matter disintegrity in Alzheimer's dementia. Thrombosis and haemostasis 114, 1230-1240 (2015).
23. Yousef, H.. et al. Aged blood impairs hippocampal neural precursor activity and activates microglia via brain endothelial cell VCAM1. Nature medicine 25, 988-1000 (2019).
24. Chakrabarty, P., et al. TLR5 decoy receptor as a novel anti-amyloid therapeutic for Alzheimer's disease. Journal of Experimental Medicine 215, 2247-2264 (2018).
25. Liu, Y.-L., et al. Amelioration of amyloid-β-induced deficits by DcR3 in an Alzheimer's disease model. Molecular neurodegeneration 12, 1-17 (2017).
26. Griesenauer, B. & Paczesny, S. The ST2/IL-33 axis in immune cells during inflammatory diseases. Frontiers in immunology 8, 475 (2017).
27. Yasuoka, S., et al. Production and functions of IL-33 in the central nervous system. Brain research 1385, 8-17 (2011).
28. Fu, A. K., et al. IL-33 ameliorates Alzheimer's disease-like pathology and cognitive decline. Proceedings of the National Academy of Sciences 113, E2705-E2713 (2016).
29. Lau, S.-F., et al. IL-33-PU. 1 Transcriptome Reprogramming Drives Functional State Transition and Clearance Activity of Microglia in Alzheimer's Disease. Cell Reports 31, 107530 (2020).
30. Trajkovic, V., Sweet, M. J. & Xu, D. T1/ST2—an IL-1 receptor-like modulator of immune responses. Cytokine & growth factor reviews 15, 87-95 (2004).
31. Kakkar, R. & Lee, R. T. The IL-33/ST2 pathway: therapeutic target and novel biomarker. Nature reviews Drug discovery 7, 827-840 (2008).
32. Homsak, E. & Gruson, D. Soluble ST2: A complex and diverse role in several diseases. Clinica Chimica Acta (2020).
33. Oshikawa, K., et al. Elevated soluble ST2 protein levels in sera of patients with asthma with an acute exacerbation. American journal of respiratory and critical care medicine 164, 277-281 (2001).
34. Bergis, D., Kassis, V. & Radeke, H. H. High plasma sST2 levels in gastric cancer and their association with metastatic disease. Cancer Biomarkers 16, 117-125 (2016).
35. Villacorta, H. & Maisel, A. S. Soluble ST2 testing: a promising biomarker in the management of heart failure. Arquivos brasileiros de cardiologia 106, 145-152 (2016).
36. Saresella, M., et al. IL-33 and its decoy sST2 in patients with Alzheimer's disease and mild cognitive impairment. Journal ofNeuroinflammation 17, 1-10 (2020).
37. Weinberg, E. O., et al. Identification of serum soluble ST2 receptor as a novel heart failure biomarker. Circulation 107, 721-726 (2003).
38. Preische, O., et al. Serum neurofilament dynamics predicts neurodegeneration and clinical progression in presymptomatic Alzheimer's disease. Nature medicine 25, 277-283 (2019).
39. Karikari, T. K., et al. Blood phosphorylated tau 181 as a biomarker for Alzheimer's disease: a diagnostic performance and prediction modelling study using data from four prospective cohorts. The Lancet Neurology 19, 422-433 (2020).
40. Gate, D., et al. Clonally expanded CD8 T cells patrol the cerebrospinal fluid in Alzheimer's disease. Nature 577, 399-404 (2020).
41. Yuan, P., et al. TREM2 haplodeficiency in mice and humans impairs the microglia barrier function leading to decreased amyloid compaction and severe axonal dystrophy. Neuron 90, 724-739 (2016).
42. Lehallier, B., et al. Undulating changes in human plasma proteome profiles across the lifespan. Nature medicine 25, 1843-1850 (2019).
43. Sasayama, D., et al. Increased cerebrospinal fluid interleukin-6 levels in patients with schizophrenia and those with major depressive disorder. Journal of psychiatric research 47, 401-406 (2013).
44. Ho, J. E., et al. Common genetic variation at the IL1RL1 locus regulates IL-33/ST2 signaling. The Journal of clinical investigation 123, 4208-4218 (2013).
45. Grotenboer, N. S., Ketelaar, M. E., Koppelman, G. H. & Nawijn, M. C. Decoding asthma: translating genetic variation in IL33 and IL1RL1 into disease pathophysiology. Journal of allergy and clinical immunology 131, 856-865. e859 (2013).
46. Zhou, X., et al. Non-coding variability at the APOE locus contributes to the Alzheimer's risk. Nature communications 10, 1-16 (2019).
47. Hormozdiari, F., Kostem, E., Kang, E. Y., Pasaniuc, B. & Eskin, E. Identifying causal variants at loci with multiple signals of association. Genetics 198, 497-508 (2014).
48. Lonsdale, J., et al. The genotype-tissue expression (GTEx) project. Nature genetics 45, 580-585 (2013).
49. Consortium, G. The Genotype-Tissue Expression (GTEx) pilot analysis: Multitissue gene regulation in humans. Science 348, 648-660 (2015).
50. Lau, S.-F., Cao, H., Fu, A. K. & Ip, N. Y. Single-nucleus transcriptome analysis reveals dysregulation of angiogenic endothelial cells and neuroprotective glia in Alzheimer's disease. Proceedings of the National Academy of Sciences 117, 25800-25809 (2020).
51. Li, W., Notani, D. & Rosenfeld, M. G. Enhancers as non-coding RNA transcription units: recent insights and future perspectives. Nature Reviews Genetics 17, 207 (2016).
52. Zhou, X., et al. Identification of genetic risk factors in the Chinese population implicates a role of immune system in Alzheimer's disease pathogenesis. Proceedings of the National Academy of Sciences 115, 1697-1706 (2018).
53. Lee, J. H., Cheng, R., Graff-Radford, N., Foroud, T. & Mayeux, R. Analyses of the national institute on aging late-onset alzheimer's disease family study: implication of additional loci. Archives of neurology 65, 1518-1526 (2008).
54. Naj, A. C., et al. Common variants at MS4A4/MS4A6E, CD2AP, CD33 and EPHA1 are associated with late-onset Alzheimer's disease. Nature genetics 43, 436-441 (2011).
55. Jun, G.. et al. Meta-analysis confirms CR1, CLU, and PICALM as alzheimer disease risk loci and reveals interactions with APOE genotypes. Archives of neurology 67, 1473-1484 (2010).
56. Kathryn, A. E., et al. The Australian Imaging, Biomarkers and Lifestyle (AIBL) study of aging: methodology and baseline characteristics of 1112 individuals recruited for a longitudinal study of Alzheimer's disease. International Psychogeriatrics (2009).
57. Keren-Shaul, H.. et al. A unique microglia type associated with restricting development of Alzheimer's disease. Cell 169, 1276-1290. e1217 (2017).
58. Thrupp, N., et al. Single-nucleus RNA-Seq is not suitable for detection of microglial activation genes in humans. Cell reports 32, 108189 (2020).
59. Ungar, L., Altmann, A. & Greicius, M. D. Apolipoprotein E, gender, and Alzheimer's disease: an overlooked, but potent and promising interaction. Brain imaging and behavior 8, 262-273 (2014).
60. Wang, Y., et al. Astrocyte-secreted IL-33 mediates homeostatic synaptic plasticity in the adult hippocampus. Proceedings of the National Academy of Sciences 118(2021).
61. Nguyen, P. T., et al. Microglial remodeling of the extracellular matrix promotes synapse plasticity. Cell 182, 388-403. e315 (2020).
62. Hansen, D. V., Hanson, J. E. & Sheng, M. Microglia in Alzheimer's disease. Journal of Cell Biology 217, 459-472 (2018).
63. Mosher, K. I. & Wyss-Coray, T. Microglial dysfunction in brain aging and Alzheimer's disease. Biochemical pharmacology 88, 594-604 (2014).
64. Bartunek, J., et al. Nonmyocardial production of ST2 protein in human hypertrophy and failure is related to diastolic load. Journal of the American College of Cardiology 52, 2166-2174 (2008).
65. Houghton-Trivino, N., Salgado, D. M., Rodriguez, J. A., Bosch, I. & Castellanos, J. E. Levels of soluble ST2 in serum associated with severity of dengue due to tumour necrosis factor alpha stimulation. Journal of General Virology 91, 697-706 (2010).
66. Mildner, M., et al. Primary sources and immunological prerequisites for sST2 secretion in humans. Cardiovascular research 87, 769-777 (2010).
67. Tabruyn, S. P. & Griffioen, A. W. NF-κB: a new player in angiostatic therapy. Angiogenesis 11, 101-106 (2008).
68. Zlokovic, B. V. Neurovascular pathways to neurodegeneration in Alzheimer's disease and other disorders. Nature Reviews Neuroscience 12, 723-738 (2011).
69. Bell, R. D.. et al. Apolipoprotein E controls cerebrovascular integrity via cyclophilin A. Nature 485, 512-516 (2012).
70. Montagne, A., et al. APOE4 leads to blood-brain barrier dysfunction predicting cognitive decline. Nature 581, 71-76 (2020).
71. Mhatre, M., et al. lbrombin, a mediator of neurotoxicity and memory impairment. Neurobiology of aging 25, 783-793 (2004).
72. Chen, Z.-L. & Strickland, S. Neuronal death in the hippocampus is promoted by plasmin-catalyzed degradation of laminin. Cell 91, 917-925 (1997).
73. Hilscher, M. B., et al. Mechanical stretch increases expression of CXCL1 in liver sinusoidal endothelial cells to recruit neutrophils, generate sinusoidal microthombi, and promote portal hypertension. Gastroenterology 157, 193-209. e199 (2019).
74. Grammas, P. & Ovase, R. Inflammatory factors are elevated in brain microvessels in Alzheimer's disease. Neurobiology of aging 22, 837-842 (2001).
75. Grammas, P. & Ovase, R. Cerebrovascular transforming growth factor-O contributes to inflammation in the Alzheimer's disease brain. The American journal of pathology 160, 1583-1587 (2002).
76. Huang, Y. & Mahley, R W. Apolipoprotein E: structure and function in lipid metabolism, neurobiology, and Alzheimer's diseases. Neurobiology of disease 72, 3-12 (2014).
77. Fernandez, C. G., Hamby, M. E., McReynolds, M. L. & Ray, W. J. The role of APOE4 in disrupting the homeostatic functions of astrocytes and microglia in aging and Alzheimer's disease. Frontiers in aging neuroscience 11, 14 (2019).
78. Fourgeaud, L., et al. TAM receptors regulate multiple features of microglial physiology. Nature 532, 240-244 (2016).
79. Kim, J., Basak, J. M. & Holtzman, D. M. The role of apolipoprotein E in Alzheimer's disease. Neuron 63, 287-303 (2009).
80. Wang, Y., et al. TREM2 lipid sensing sustains the microglial response in an Alzheimer's disease model. Cell 160, 1061-1071 (2015).
81. Mittal, K., et al. CD4 T cells induce a subset of MHCII-expressing microglia that attenuates Alzheimer pathology. Iscience 16, 298-311 (2019).
82. Miller, A. M., et al. IL-33 reduces the development of atherosclerosis. The Journal of experimental medicine 205, 339-346 (2008).
83. Yvan-Charvet, L., Wang, N. & Tall, A. R. Role of HDL, ABCA1, and ABCG1 transporters in cholesterol efflux and immune responses. Arteriosclerosis, thrombosis, and vascular biology 30, 139-143 (2010).
84. Lanfranco, M. F., Ng, C. A. & Rebeck, G. W. ApoE Lipidation as a Therapeutic Target in Alzheimer's Disease. International Journal of Molecular Sciences 21, 6336 (2020).
85. Horiuchi, Y., et al. Characterization of the cholesterol efflux of apolipoprotein E-containing high-density lipoprotein in THP-1 cells. Biological chemistry 400, 209-218 (2019).
86. Jiang, Q., et al. ApoE promotes the proteolytic degradation of Aβ. Neuron 58, 681-693 (2008).
87. Boehm-Cagan, A.. et al. Differential effects of apoE4 and activation of ABCA1 on brain and plasma lipoproteins. PLoS One 11, e0166195 (2016).
88. Hanson, A. J., et al. Effect of apolipoprotein E genotype and diet on apolipoprotein E lipidation and amyloid peptides: randomized clinical trial. JAMA neurology 70, 972-980 (2013).
89. Baba, Y., et al. Involvement of PU. 1 in mast cell/basophil-specific function of the human IL1RL1/ST2 promoter. Allergology International 61,461-467 (2012).
90. Xu, H., Turnquist, H. R., Hoffman, R. & Billiar, T. R. Role of the IL-33-ST2 axis in sepsis. Military Medical Research 4, 1-9 (2017).
91. Demyanets, S., et al. Components of the interleukin-33/ST2 system are differentially expressed and regulated in human cardiac cells and in cells of the cardiac vasculature. Journal of molecular and cellular cardiology 60, 16-26 (2013).
92. O'Meara, E., et al. Independent prognostic value of serum soluble ST2 measurements in patients with heart failure and a reduced ejection fraction in the PARADIGM-HF trial (prospective comparison of ARNI with ACEI to determine impact on global mortality and morbidity in heart failure). Circulation: Heart Failure 11, e004446 (2018).
93. Bartemes, K., Chen, C.-C., Iijima, K., Drake, L. & Kita, H. IL-33-responsive group 2 innate lymphoid cells are regulated by female sex hormones in the uterus. The Journal of Immunology 200, 229-236 (2018).
94. Russi, A. E., Ebel, M. E., Yang, Y. & Brown, M. A. Male-specific IL-33 expression regulates sex-dimorphic EAE susceptibility. Proceedings of the National Academy of Sciences 115, E1520-E1529 (2018).
95. Chapuis, J., et al. Transcriptomic and genetic studies identify IL-33 as a candidate gene for Alzheimer's disease. Molecular psychiatry 14, 1004-1016 (2009).
96. Association, A. P. Diagnostic and statistical manual of mental disorders (DSM-S®), (American Psychiatric Pub, 2013).
97. Pangman, V. C., Sloan, J. & Guse, L. An examination of psychometric properties of the mini-mental state examination and the standardized mini-mental state examination: implications for clinical practice. Applied Nursing Research 13, 209-213 (2000).
98. Nasreddine, Z. S., et al. The Montreal Cognitive Assessment, MoCA: a brief screening tool for mild cognitive impairment. Journal of the American Geriatrics Society 53, 695-699 (2005).
99. Purcell, S., et al. PLINK: a tool set for whole-genome association and population-based linkage analyses. The American journal of human genetics 81, 559-575 (2007).
100. Gromping, U. Relative importance for linear regression in R: the package relaimpo. Journal of statistical software 17, 1-27 (2006).
101. Patin, E., et al. Natural variation in the parameters of innate immune cells is preferentially driven by genetic factors. Nature immunology 19, 302-314 (2018).
102. Huang, D. W., Sherman, B. T. & Lempicki, R. A. Bioinformatics enrichment tools: paths toward the comprehensive functional analysis of large gene lists. Nucleic acids research 37, 1-13 (2009).
103. Sherman, B. T. & Lempicki, R. A. Systematic and integrative analysis of large gene lists using DAVID bioinformatics resources. Nature protocols 4, 44 (2009).
104. Han, B. & Eskin, E. Random-effects model aimed at discovering associations in meta-analysis of genome-wide association studies. The American Journal of Human Genetics 88, 586-598 (2011).
105. Youden, W. J. Index for rating diagnostic tests. Cancer 3, 32-35 (1950).

SEQUENCE LISTING

SEQ ID NO: 1: sgRNA-1
TTATGGACAGAATTAAGAAG
SEQ ID NO: 2: sgRNA-2,
CTGTCCATAAGATTTGAAAG
SEQ ID NO: 3: sgRNA-3
AATTTTGTTCTGGTAGCCAT
SEQ ID NO: 4: sgRNA-4
GGTATTTCAGCTAGTGCCTA

TABLE 1

| | | | | sST2 (ENST00000311734.2)^{a} | | | | ST2L (ENST00000233954.1)^{a} | | |
|---|---|---|---|---|---|---|---|---|---|---|
| | | | | Avg. | | | | Avg. | | |
| | Tissue | Tissue | | transcript | | | | transcript | | |
| # | type | subregion | n | level^{b} | β (SE) | | P | level^{b} | β (SE) | P |
| 1 | Adipose | Visceral | 213 | 11.485 | 0.035 | (0.106) | 0.741 | 0.270 | −0.087 (0.107) | 0.418 |
| | | (omentum) | | | | | | | | |
| 2 | | Subcutaneous | 332 | 8.333 | 0.023 | (0.077) | 0.762 | — | — | — |
| 3 | Adrenal | Adrenal gland | 148 | 23.138 | 0.163 | (0.135) | 0.228 | — | — | — |
| | gland | | | | | | | | | |
| 4 | Artery | Aorta | 226 | 3.024 | −0.091 | (0.098) | 0.355 | — | — | — |
| 5 | | Tibial | 319 | 1.168 | −0.053 | (0.080) | 0.508 | — | — | — |
| 6 | | Coronary | 124 | 4.329 | 0.065 | (0.142) | 0.647 | — | — | — |
| 7 | Brain | Anterior cingulate | 86 | 3.156 | −0.179 | (0.153) | 0.241 | — | — | — |
| | | cortex (BA24) | | | | | | | | |
| 8 | | Caudate (basal | 119 | 2.461 | −0.265 | (0.135) | 0.051 | — | — | — |
| | | ganglia) | | | | | | | | |
| 9 | | Cerebellar | 104 | 1.031 | −0.194 | (0.134) | 0.148 | — | — | — |
| | | hemisphere | | | | | | | | |
| 10 | | Cerebellum | 121 | 1.229 | −0.309 | (0.121) | 0.011 | — | — | — |
| 11 | | Cortex | 110 | 2.576 | −0.275 | (0.135) | 0.046 | — | — | — |
| 12 | | Amygdala | 69 | 2.688 | −0.388 | (0.176) | 0.031 | — | — | — |
| 13 | | Frontal Cortex | 108 | 2.848 | −0.319 | (0.143) | 0.027 | — | — | — |
| | | (BA9) | | | | | | | | |
| 14 | | Spinal cord | 67 | 3.306 | −0.521 | (0.184) | 0.006 | — | — | — |
| | | (cervical c-1) | | | | | | | | |
| 15 | | Hippocampus | 92 | 2.226 | −0.327 | (0.161) | 0.045 | — | — | — |
| 16 | | Nucleus | 108 | 2.317 | −0.390 | (0.145) | 0.008 | — | — | — |
| | | accumbens (basal | | | | | | | | |
| | | ganglia) | | | | | | | | |
| 17 | | Substantia nigra | 64 | 5.066 | −0.344 | (0.155) | 0.029 | — | — | — |
| 18 | | Putamen (basal | 91 | 2.205 | −0.290 | (0.131) | 0.030 | — | — | — |
| | | ganglia) | | | | | | | | |
| 19 | | Hypothalamus | 91 | 3.348 | −0.258 | (0.142) | 0.071 | — | — | — |
| 20 | Breast | Mammary tissue | 193 | 8.720 | −0.036 | (0.096) | 0.705 | — | — | — |
| 21 | Cell line | Transformed | 273 | 4.102 | −0.034 | (0.084) | 0.684 | — | — | — |
| | | fibroblasts | | | | | | | | |
| 22 | | EBV-transformed | 120 | — | — | | — | — | — | — |
| | | lymphocytes | | | | | | | | |
| 23 | Colon | Transverse | 185 | 3.311 | −0.127 | (0.110) | 0.249 | — | — | — |
| 24 | | Sigmoid | 159 | 2.043 | 0.027 | (0.122) | 0.820 | — | — | — |
| 25 | Esophagus | Gastroesophageal | 155 | 3.690 | −0.188 | (0.120) | 0.120 | 0.327 | −0.056 (0.097) | 0.565 |
| | | junction | | | | | | | | |
| 26 | | Muscularis | 260 | 3.059 | −0.105 | (0.087) | 0.230 | 0.301 | 0.010 (0.078) | 0.900 |
| 27 | | Mucosa | 300 | 1.464 | −0.090 | (0.092) | 0.326 | — | — | — |
| 28 | Heart | Left ventricle | 240 | 7.199 | −0.022 | (0.101) | 0.824 | — | — | — |
| 29 | | Atrial appendage | 194 | 8.955 | 0.017 | (0.117) | 0.885 | — | — | — |
| 30 | Liver | Liver | 120 | 9.797 | 0.169 | (0.146) | 0.251 | — | — | — |
| 31 | Lung | Lung | 331 | 98.940 | −0.239 | (0.077) | 0.002 | 1.168 | −0.021 (0.074) | 0.772 |
| 32 | Minor | Minor salivary | 65 | 1.333 | −0.219 | (0.216) | 0.316 | — | — | — |
| | salivary | gland | | | | | | | | |
| | gland | | | | | | | | | |
| 33 | Muscle | Skeletal muscle | 410 | 0.935 | −0.113 | (0.068) | 0.099 | — | — | — |
| 34 | Nerve | Tibial nerve | 294 | 2.165 | −0.036 | (0.084) | 0.669 | — | — | — |
| 35 | Ovary | Ovary | 100 | 0.895 | −0.037 | (0.152) | 0.805 | — | — | — |
| 36 | Pancreas | Pancreas | 175 | 1.905 | 0.009 | (0.116) | 0.937 | — | — | — |
| 37 | Pituitary | Pituitary | 109 | 4.065 | −0.059 | (0.126) | 0.635 | — | — | — |
| 38 | Prostate | Prostate | 105 | 3.961 | −0.082 | (0.137) | 0.548 | — | — | — |
| 39 | Skin | Not sun exposed | 239 | 2.698 | 0.116 | (0.105) | 0.269 | — | — | — |
| | | (suprapubic) skin | | | | | | | | |
| 40 | | Sun exposed | 348 | 3.648 | 0.006 | (0.078) | 0.929 | — | — | — |
| | | (lower leg) skin | | | | | | | | |
| 41 | Small | Terminal ileum | 94 | 4.894 | −0.172 | (0.147) | 0.245 | — | — | — |
| | intestine | | | | | | | | | |
| 42 | Spleen | Spleen | 110 | 0.937 | 0.153 | (0.142) | 0.282 | — | — | — |
| 43 | Stomach | Stomach | 189 | 11.447 | −0.025 | (0.143) | 0.860 | — | — | — |
| 44 | Testis | Testis | 177 | 0.623 | −0.137 | (0.111) | 0.220 | — | — | — |
| 45 | Thyroid | Thyroid | 322 | 2.764 | −0.013 | (0.079) | 0.867 | — | — | — |
| 46 | Uterus | Uterus | 83 | 1.698 | −0.081 | (0.155) | 0.617 | — | — | — |
| 47 | Vagina | Vagina | 90 | 2.105 | −0.080 | (0.146) | 0.584 | — | — | — |
| 48 | Whole | Whole blood | 403 | 1.858 | 0.081 | (0.076) | 0.286 | — | — | — |
| | blood | | | | | | | | | |

TABLE 2

| | | Healthy control | | AD | |
|---|---|---|---|---|---|
| Sample size (n) | | 345 | | 345 | |
| Basic | Age, years (SD) | 73.12 | (4.71) | 80.53 | (5.96) |
| information | Sex, male (%) | 140 | (40.58%) | 110 | (31.88%) |
| | Education level, years (SD) | 8.16 | (5.06) | 4.85 | (4.70) |
| | MoCA score (SD) | 23.62 | (3.31) | 12.61 | (5.42) |
| | APOE-ε4, allele frequency | 7.97% | | 19.71% | |
| | APOE-ε2, allele frequency | 10.43% | | 5.07% | |
| CVD records | Heart disease (%) | 30 | (8.70%) | 57 | (16.52%) |
| | Hypertension (%) | 211 | (61.16%) | 207 | (60.00%) |
| | Diabetes Mellitus (%) | 76 | (22.03%) | 106 | (30.72%) |
| | Hyperlipidemia (%) | 104 | (30.14%) | 146 | (42.32%) |
| | No CVDs (%) | 97 | (28.12%) | 86 | (24.93%) |
| Data | Plasma sST2 (mean level) | 336 | (12.30 ng/mL) | 277 | (14.09 ng/mL) |
| available (n) | Plasma P-tau181 (mean | 134 | (2.35 pg/mL) | 156 | (4.10 pg/mL) |
| | level) | | | | |
| | Plasma NfL (mean level) | 135 | (16.19 pg/mL) | 154 | (29.12 pg/mL) |
| | Gray matter MRI (mean | 115 | (40.75%) | 77 | (39.44%) |

| | | |
|---|---|---|
| % ICV) | | |
| Genotypes (WGS) | 194 | 233 |
| Genotypes (APOE-ε2, | 151 | 112 |
| APOE-ε4, rs1921622 array) | | |

TABLE 3

| | | Healthy control | | AD | |
|---|---|---|---|---|---|
| Sample size (n) | | 11 | | 102 | |
| Basic | Age, years (SD) | 89.82 | (4.98) | 79.96 | (9.89) |
| information | Sex, male (%) | 4 | (36.36%) | 55 | (53.92%) |
| | PMD, hours (SD) | 22.77 | (6.55) | 26.15 | (18.00) |
| | APOE-ε4, allele frequency | 9.09% | | 38.24% | |
| | APOE-ε2, allele frequency | 18.18% | | 4.41% | |
| Data | CSF sST2 (mean level) | 11 | (2.01 ng/mL) | 75 | (6.55 ng/mL) |

| | | | |
|---|---|---|---|
| available (n) | Prefrontal cortex | 11 | 78 |
| | slice for staining | | |
| | Single-nuclei RNA-seq | 9 | 12 |
| | Genotypes (APOE-ε2, | | |
| | APOE-ε4, rs1921622 | 11 | 102 |
| | array) | | |

TABLE 4

| | | Effective | Esti- | | |
|---|---|---|---|---|---|
| POS | SNP | Allele | mate | Stat | p-value |
| 1: 163472218 | rs75687525 | C | 2.38 | 4.55 | 7.14E−06 |
| 10: 27968343 | rs1907370 | T | 2.59 | 4.65 | 4.37E−06 |
| 12: 107400417 | rs66994203 | G | 3.62 | 5.45 | 8.68E−08 |
| 12: 74877668 | rs35957931 | A | 2.33 | 4.57 | 6.47E−06 |
| 12: 74879965 | rs1824539 | T | 2.34 | 4.54 | 7.27E−06 |
| 12: 74889088 | rs77474403 | G | 2.34 | 4.54 | 7.27E−06 |
| 12: 74891319 | rs7300059 | G | 2.38 | 4.6 | 5.52E−06 |
| 12: 74892234 | rs7304361 | G | 2.43 | 4.63 | 4.81E−06 |
| 12: 74900231 | rs202095841 | A | 2.42 | 4.67 | 4.09E−06 |
| 12: 74901707 | rs147414753 | T | 2.34 | 4.54 | 7.27E−06 |
| 12: 74902303 | rs12821034 | G | 2.36 | 4.53 | 7.82E−06 |
| 12: 74902947 | rs36153793 | C | 2.34 | 4.54 | 7.27E−06 |
| 12: 74908207 | rs7962722 | T | 2.34 | 4.54 | 7.27E−06 |
| 12: 74910670 | rs12816163 | T | 2.34 | 4.54 | 7.27E−06 |
| 12: 74914614 | rs4547177 | C | 2.34 | 4.54 | 7.27E−06 |
| 12: 74914810 | rs2365919 | G | 2.34 | 4.54 | 7.27E−06 |
| 12: 74915296 | rs35454023 | A | 2.34 | 4.54 | 7.27E−06 |
| 12: 74915984 | rs58783285 | T | 2.34 | 4.54 | 7.27E−06 |
| 12: 74917553 | rs1826588 | A | 2.34 | 4.54 | 7.27E−06 |
| 12: 74920470 | rs1493810 | G | 2.34 | 4.54 | 7.27E−06 |
| 12: 74922159 | rs34852307 | G | 2.33 | 4.49 | 9.31E−06 |
| 12: 74922723 | rs2605342 | T | −2.34 | −4.54 | 7.27E−06 |
| 12: 74923340 | rs2605340 | A | −2.34 | −4.54 | 7.27E−06 |
| 12: 74923633 | rs12821741 | G | 2.34 | 4.54 | 7.27E−06 |
| 12: 74924552 | rs1493801 | C | 2.34 | 4.54 | 7.27E−06 |
| 12: 74925689 | rs1461045 | T | −2.34 | −4.54 | 7.27E−06 |
| 12: 74926002 | rs1493800 | T | 2.34 | 4.54 | 7.27E−06 |
| 12: 74926682 | rs17113674 | C | 2.34 | 4.54 | 7.27E−06 |
| 12: 74926988 | rs17113675 | T | 2.34 | 4.54 | 7.27E−06 |
| 12: 74927228 | rs17113679 | A | 2.34 | 4.54 | 7.27E−06 |
| 12: 74927335 | rs60905363 | C | 2.34 | 4.54 | 7.27E−06 |
| 12: 74928312 | rs34706013 | A | 2.34 | 4.54 | 7.27E−06 |
| 12: 74929829 | rs2126136 | T | 2.34 | 4.54 | 7.27E−06 |
| 12: 74929925 | rs2605381 | G | −2.34 | −4.54 | 7.27E−06 |
| 12: 74930524 | rs12832045 | A | 2.34 | 4.54 | 7.27E−06 |
| 12: 74931349 | rs1493796 | G | 2.34 | 4.54 | 7.27E−06 |
| 12: 74932159 | rs590352 | C | −2.34 | −4.54 | 7.27E−06 |
| 12: 74947219 | rs11615704 | T | 2.43 | 4.72 | 3.17E−06 |
| 13: 24695615 | rs117523785 | G | 3.47 | 4.52 | 8.23E−06 |
| 15: 91664657 | rs17641976 | A | 4.28 | 5.14 | 4.21E−07 |
| 15: 91665392 | rs17515887 | C | 3.87 | 4.71 | 3.35E−06 |
| 17: 13640730 | rs7208104 | C | −2.18 | −4.78 | 2.46E−06 |
| 17: 13645480 | rs8081904 | G | −2.09 | −4.55 | 7.10E−06 |
| 17: 70243206 | rs12600563 | T | 2.43 | 4.56 | 6.74E−06 |
| 19: 54170960 | rs142226688 | A | 3.5 | 4.64 | 4.72E−06 |
| 2: 102863095 | rs56238602 | T | 1.77 | 4.56 | 6.63E−06 |
| 2: 102917234 | rs1420091 | C | −2.78 | −8.07 | 7.52E−15 |
| 2: 102917788 | rs4399750 | C | −2.86 | −8.17 | 3.75E−15 |
| 2: 102918018 | rs4577297 | A | −3.38 | −10.08 | 1.59E−21 |
| 2: 102918601 | rs2110660 | G | −2.86 | −8.21 | 2.74E−15 |
| 2: 102919305 | rs1420090 | C | −2.82 | −8.07 | 7.80E−15 |
| 2: 102919426 | rs7565653 | A | −2.82 | −8.1 | 6.36E−15 |
| 2: 102920037 | rs7568913 | C | −3.33 | −10.06 | 1.78E−21 |
| 2: 102921783 | rs10179654 | G | −3.27 | −9.8 | 1.53E−20 |
| 2: 102922987 | rs4090473 | G | −2.85 | −8.22 | 2.57E−15 |
| 2: 102924549 | rs12476925 | T | −2.85 | −8.22 | 2.57E−15 |
| 2: 102924662 | rs12476968 | T | −2.85 | −8.22 | 2.57E−15 |
| 2: 102924947 | rs7562254 | C | −3.33 | −10.02 | 2.56E−21 |
| 2: 102925223 | rs56224165 | T | −2.72 | −7.63 | 1.65E−13 |
| 2: 102925790 | rs4851564 | C | −2.73 | −7.69 | 1.10E−13 |
| 2: 102926320 | rs6721346 | C | −2.82 | −8.15 | 4.40E−15 |
| 2: 102926362 | rs12470864 | A | −2.88 | −8.4 | 7.15E−16 |
| 2: 102926366 | rs12470867 | A | −2.7 | −7.56 | 2.55E−13 |
| 2: 102926511 | rs10178436 | C | −3.31 | −9.94 | 5.03E−21 |
| 2: 102926800 | rs11679191 | T | −2.82 | −8.15 | 4.40E−15 |
| 2: 102926981 | rs11685424 | A | −2.82 | −8.15 | 4.40E−15 |
| 2: 102927086 | rs11685480 | A | −2.82 | −8.15 | 4.40E−15 |
| 2: 102927641 | rs6543115 | G | −3.35 | −10.05 | 2.03E−21 |
| 2: 102927726 | rs6543116 | G | −3.38 | −10.11 | 1.27E−21 |
| 2: 102929012 | rs6733174 | C | −2.79 | −8.04 | 9.39E−15 |
| 2: 102929469 | rs6543118 | A | −3.35 | −10.03 | 2.46E−21 |
| 2: 102930147 | rs1558622 | A | −2.86 | −8.21 | 2.77E−15 |
| 2: 102930307 | rs1558621 | G | −2.85 | −8.23 | 2.41E−15 |
| 2: 102930380 | rs10189202 | G | −2.86 | −8.21 | 2.77E−15 |
| 2: 102930657 | rs10191914 | C | −2.86 | −8.21 | 2.77E−15 |
| 2: 102930881 | rs10189711 | G | −2.9 | −8.36 | 9.65E−16 |
| 2: 102930948 | rs12712135 | G | −3.35 | −10.03 | 2.46E−21 |
| 2: 102931395 | rs1558620 | C | −2.86 | −8.21 | 2.77E−15 |
| 2: 102931550 | rs1558619 | T | −2.81 | −8.08 | 7.12E−15 |
| 2: 102931802 | rs12996505 | G | −3.34 | −9.99 | 3.32E−21 |
| 2: 102931826 | rs13020553 | G | −2.91 | −8.46 | 4.75E−16 |
| 2: 102931926 | rs13020793 | T | −2.86 | −8.21 | 2.77E−15 |
| 2: 102932247 | rs10183388 | T | −3.34 | −9.99 | 3.32E−21 |
| 2: 102932293 | rs953934 | T | −3.34 | −9.99 | 3.32E−21 |
| 2: 102932562 | rs950880 | A | −2.91 | −8.46 | 4.75E−16 |
| 2: 102933552 | rs1968171 | T | −2.86 | −8.21 | 2.77E−15 |
| 2: 102933758 | rs4613307 | G | −2.84 | −8.11 | 5.83E−15 |
| 2: 102933802 | rs1968170 | A | −2.78 | −8 | 1.26E−14 |
| 2: 102935237 | rs11123918 | C | −2.86 | −8.21 | 2.77E−15 |
| 2: 102935805 | rs10182639 | A | −2.86 | −8.21 | 2.77E−15 |
| 2: 102935974 | rs11693204 | A | −2.67 | −7.48 | 4.54E−13 |
| 2: 102936131 | rs11690443 | A | −2.86 | −8.18 | 3.47E−15 |
| 2: 102936366 | rs12712136 | C | −2.86 | −8.18 | 3.47E−15 |
| 2: 102936981 | rs974389 | A | −2.86 | −8.18 | 3.47E−15 |
| 2: 102937482 | rs4142132 | A | −2.86 | −8.18 | 3.47E−15 |
| 2: 102938335 | rs971764 | T | −2.86 | −8.18 | 3.47E−15 |
| 2: 102939036 | rs13001325 | T | −2.89 | −8.39 | 7.72E−16 |
| 2: 102939434 | rs1420088 | C | −2.86 | −8.18 | 3.47E−15 |
| 2: 102939709 | rs66780767 | T | −2.23 | −6.3 | 7.74E−10 |
| 2: 102939754 | rs11123919 | A | −2.91 | −8.36 | 9.17E−16 |
| 2: 102939833 | rs11123920 | T | −2.85 | −8.16 | 4.07E−15 |
| 2: 102940412 | rs6706844 | C | −2.83 | −8.12 | 5.50E−15 |
| 2: 102940999 | rs11674832 | G | −2.82 | −8.04 | 9.36E−15 |
| 2: 102941009 | rs11679889 | A | −2.65 | −7.39 | 8.11E−13 |
| 2: 102941019 | rs11675988 | C | −2.82 | −8.04 | 9.36E−15 |
| 2: 102941074 | rs11679900 | T | −2.82 | −8.04 | 9.36E−15 |
| 2: 102941215 | rs11676075 | C | −2.82 | −8.04 | 9.36E−15 |
| 2: 102941338 | rs11676124 | C | −2.85 | −8.11 | 5.89E−15 |
| 2: 102941561 | rs11123921 | G | −2.86 | −8.18 | 3.47E−15 |
| 2: 102941715 | rs12992762 | C | −2.86 | −8.18 | 3.47E−15 |
| 2: 102941818 | rs12998412 | C | −2.86 | −8.18 | 3.47E−15 |
| 2: 102941918 | rs9750771 | A | −3.38 | −10.04 | 2.22E−21 |
| 2: 102942187 | rs11123922 | C | −2.86 | −8.18 | 3.47E−15 |
| 2: 102942537 | rs12053422 | A | −2.86 | −8.18 | 3.47E−15 |
| 2: 102942920 | rs58933240 | C | −2.84 | −8.13 | 5.09E−15 |
| 2: 102942921 | rs56403447 | C | −2.84 | −8.13 | 5.09E−15 |
| 2: 102942939 | rs13005688 | C | −2.85 | −8.16 | 4.02E−15 |
| 2: 102943386 | rs12725988 | T | −2.84 | −8.13 | 5.09E−15 |
| 2: 102943822 | rs4490207 | G | −2.86 | −8.18 | 3.47E−15 |
| 2: 102943844 | rs4455171 | T | −2.86 | −8.18 | 3.47E−15 |
| 2: 102943883 | rs13007344 | C | −2.86 | −8.18 | 3.47E−15 |
| 2: 102944191 | rs141508079 | G | −2.11 | −5.11 | 4.86E−07 |
| 2: 102945241 | rs76520363 | A | −2.86 | −8.18 | 3.47E−15 |
| 2: 102945378 | rs76278109 | G | −2.86 | −8.18 | 3.47E−15 |
| 2: 102945416 | rs76886731 | T | −2.86 | −8.18 | 3.47E−15 |
| 2: 102945755 | rs150341880 | T | −2.83 | −8.11 | 5.62E−15 |
| 2: 102946082 | rs75392560 | G | −2.8 | −7.98 | 1.46E−14 |
| 2: 102946111 | rs76498201 | G | −2.84 | −8.07 | 7.84E−15 |
| 2: 102946677 | rs56192435 | G | −2.71 | −7.55 | 2.76E−13 |
| 2: 102947201 | rs12996772 | T | −2.86 | −8.18 | 3.47E−15 |
| 2: 102948470 | rs1420104 | A | −2.89 | −8.39 | 7.72E−16 |
| 2: 102948632 | rs1420103 | C | −3.41 | −10.09 | 1.47E−21 |
| 2: 102948819 | rs1420102 | T | −2.86 | −8.18 | 3.47E−15 |
| 2: 102948939 | rs12466380 | G | −2.81 | −7.98 | 1.48E−14 |
| 2: 102949161 | rs12479210 | T | −2.89 | −8.39 | 7.72E−16 |
| 2: 102950822 | rs13019081 | C | −2.91 | −8.38 | 8.44E−16 |
| 2: 102951062 | rs12712140 | A | −2.89 | −8.3 | 1.51E−15 |
| 2: 102951073 | rs1997467 | G | −2.86 | −8.18 | 3.47E−15 |
| 2: 102951467 | rs1997466 | G | −2.86 | −8.18 | 3.47E−15 |
| 2: 102951798 | rs1362350 | C | −2.86 | −8.18 | 3.47E−15 |
| 2: 102951972 | rs1362349 | C | −2.86 | −8.18 | 3.47E−15 |
| 2: 102952360 | rs17026974 | A | −2.65 | −7.39 | 8.26E−13 |
| 2: 102953067 | rs12712141 | C | −2.75 | −7.88 | 2.89E−14 |
| 2: 102954190 | rs59247511 | C | −2.86 | −8.18 | 3.47E−15 |
| 2: 102955683 | rs873022 | T | −2.6 | −7.25 | 2.05E−12 |
| 2: 102955860 | rs3771177 | T | −2.7 | −7.51 | 3.60E−13 |
| 2: 102957532 | rs3732129 | C | −2.7 | −7.51 | 3.60E−13 |
| 2: 102957716 | rs1420101 | T | −2.9 | −8.4 | 6.87E−16 |
| 2: 102960007 | rs12905 | A | −2.71 | −7.55 | 2.76E−13 |
| 2: 102960281 | rs3821204 | G | −2.71 | −7.55 | 2.76E−13 |
| 2: 102960485 | rs13001714 | G | −2.89 | −8.39 | 7.72E−16 |
| 2: 102960584 | rs12712142 | A | −2.92 | −8.47 | 4.41E−16 |
| 2: 102963072 | rs6543119 | T | −2.9 | −8.43 | 5.91E−16 |
| 2: 102964742 | rs13017455 | T | −2.92 | −8.47 | 4.41E−16 |
| 2: 102964861 | rs55927292 | T | −2.71 | −7.55 | 2.76E−13 |
| 2: 102965332 | rs17027006 | C | −2.71 | −7.55 | 2.76E−13 |
| 2: 102965871 | rs12469506 | T | −2.71 | −7.55 | 2.76E−13 |
| 2: 102966067 | rs1921622 | A | −3.35 | −10.21 | 5.35E−22 |
| 2: 102966783 | rs1861246 | C | −3.38 | −10.07 | 1.65E−21 |
| 2: 102967844 | rs11123923 | A | −2.92 | −8.47 | 4.41E−16 |
| 2: 102969606 | rs35224028 | C | −2.88 | −8.42 | 5.99E−16 |
| 2: 102969807 | rs67723747 | T | −2.73 | −7.62 | 1.72E−13 |
| 2: 102971165 | rs56386507 | T | −2.67 | −7.44 | 5.91E−13 |
| 2: 102971865 | rs13015714 | T | −3.36 | −10.02 | 2.50E−21 |
| 2: 102974129 | rs12999364 | T | −2.86 | −8.24 | 2.30E−15 |
| 2: 102974417 | rs12998521 | T | −2.88 | −8.32 | 1.26E−15 |
| 2: 102975336 | rs12987977 | G | −2.86 | −8.24 | 2.30E−15 |
| 2: 102977537 | rs6710885 | G | −2.82 | −8.07 | 7.50E−15 |
| 2: 102979028 | rs2287037 | T | −2.86 | −8.24 | 2.30E−15 |
| 2: 102981644 | rs6727306 | C | −3.32 | −9.8 | 1.48E−20 |
| 2: 102982466 | rs11465583 | G | −3.27 | −9.59 | 7.91E−20 |
| 2: 102982703 | rs10197284 | A | −3.19 | −9.37 | 4.79E−19 |
| 2: 102982876 | rs1573895 | C | −3.32 | −9.86 | 9.52E−21 |
| 2: 102983247 | rs4851569 | A | −2.86 | −8.24 | 2.30E−15 |
| 2: 102984279 | rs1420098 | C | −2.85 | −8.25 | 2.12E−15 |
| 2: 102984671 | rs1882348 | A | −2.86 | −8.24 | 2.30E−15 |
| 2: 102984684 | rs1558627 | A | −3.32 | −9.86 | 9.52E−21 |
| 2: 102985424 | rs2058622 | G | −3.32 | −9.86 | 9.52E−21 |
| 2: 102985812 | rs3771172 | T | −2.56 | −7.05 | 7.38E−12 |
| 2: 102985950 | rs3771171 | C | −2.58 | −7.09 | 5.74E−12 |
| 2: 102985980 | rs3771170 | A | −3.23 | −9.51 | 1.51E−19 |
| 2: 102986154 | rs2160202 | A | −2.69 | −7.43 | 6.30E−13 |
| 2: 102986170 | rs2058623 | T | −3.28 | −9.75 | 2.31E−20 |
| 2: 102986618 | rs1465321 | C | −3.32 | −9.86 | 9.52E−21 |
| 2: 102987526 | rs6742875 | T | −3.24 | −9.52 | 1.47E−19 |
| 2: 102987698 | rs6733346 | C | −3.23 | −9.43 | 2.81E−19 |
| 2: 102989734 | rs2041740 | G | −3.24 | −9.52 | 1.47E−19 |
| 2: 102991181 | rs6731154 | T | −3.24 | −9.52 | 1.47E−19 |
| 2: 102992079 | rs2270298 | G | −2.88 | −8.11 | 5.64E−15 |
| 2: 102992675 | rs2270297 | C | −3.24 | −9.52 | 1.47E−19 |
| 2: 102993161 | rs6753717 | C | −3.24 | −9.52 | 1.47E−19 |
| 2: 102994056 | rs10190555 | G | −3.24 | −9.52 | 1.47E−19 |
| 2: 102994714 | rs6750020 | A | −3.24 | −9.52 | 1.47E−19 |
| 2: 102994884 | rs17027037 | G | −2.88 | −8.11 | 5.64E−15 |
| 2: 102995020 | rs2080289 | A | −2.88 | −8.11 | 5.64E−15 |
| 2: 102996022 | rs6729638 | T | −3.19 | −9.28 | 9.09E−19 |
| 2: 102996805 | rs11683700 | T | −2.88 | −8.11 | 5.64E−15 |
| 2: 102996872 | rs3821203 | T | −2.86 | −8.03 | 9.92E−15 |
| 2: 102997174 | rs3771162 | T | −2.88 | −8.11 | 5.64E−15 |
| 2: 102997733 | rs11465633 | T | −2.88 | −8.11 | 5.64E−15 |
| 2: 102999312 | rs56258475 | G | −2.88 | −8.11 | 5.64E−15 |
| 2: 103001402 | rs1035130 | T | −2.88 | −8.11 | 5.64E−15 |
| 2: 103002395 | rs3755274 | G | −3.24 | −9.52 | 1.47E−19 |
| 2: 103003043 | rs2241117 | C | −3.24 | −9.52 | 1.47E−19 |
| 2: 103003265 | rs2241116 | A | −2.46 | −5.97 | 5.17E−09 |
| 2: 103006387 | rs4851570 | G | −2.88 | −8.11 | 5.64E−15 |
| 2: 103007220 | rs2001461 | C | −3.24 | −9.52 | 1.47E−19 |
| 2: 103007567 | rs17027060 | C | −2.88 | −8.11 | 5.84E−15 |
| 2: 103007623 | rs3860444 | G | −3.24 | −9.52 | 1.47E−19 |
| 2: 103008710 | rs12712145 | C | −3.2 | −9.41 | 3.42E−19 |
| 2: 103008969 | rs66919607 | G | −2.74 | −7.73 | 7.92E−14 |
| 2: 103010530 | rs2287035 | A | −2.83 | −8 | 1.26E−14 |
| 2: 103010588 | rs2287034 | A | −2.83 | −8 | 1.26E−14 |
| 2: 103011552 | rs4851005 | T | −2.83 | −8 | 1.26E−14 |
| 2: 103012674 | rs17027071 | T | −2.37 | −6.8 | 3.65E−11 |
| 2: 103013962 | rs3732126 | C | −2.83 | −8 | 1.26E−14 |
| 2: 103013963 | rs3732125 | C | −2.85 | −8.02 | 1.06E−14 |
| 2: 103014302 | rs1135354 | G | −2.77 | −7.83 | 3.98E−14 |
| 2: 103014696 | rs1568681 | T | −3.2 | −9.41 | 3.42E−19 |
| 2: 103015918 | rs17027087 | T | −2.83 | −7.96 | 1.65E−14 |
| 2: 103016216 | rs55664618 | A | −2.7 | −7.43 | 6.03E−13 |
| 2: 103018077 | rs3732123 | G | −2.78 | −7.79 | 5.26E−14 |
| 2: 103019782 | rs55742125 | A | −2.83 | −7.96 | 1.65E−14 |
| 2: 103019919 | rs1035127 | G | −3.22 | −9.41 | 3.28E−19 |
| 2: 103024331 | rs55883125 | A | −2.83 | −7.96 | 1.65E−14 |
| 2: 103024738 | rs4851006 | A | −2.83 | −7.96 | 1.65E−14 |
| 2: 103024813 | rs4851007 | G | −3.25 | −9.46 | 2.23E−19 |
| 2: 103025203 | rs4851575 | A | −3.17 | −9.35 | 5.64E−19 |
| 2: 103026611 | rs4851008 | C | −3.21 | −9.39 | 3.93E−19 |
| 2: 103027103 | rs6419573 | C | −3.21 | −9.39 | 3.93E−19 |
| 2: 103029165 | rs11693955 | T | −2.84 | −7.99 | 1.35E−14 |
| 2: 103029410 | rs6543132 | G | −3.21 | −9.39 | 3.93E−19 |
| 2: 103033147 | rs1807782 | T | −3.21 | −9.39 | 3.93E−19 |
| 2: 103033825 | rs3755272 | A | −2.38 | −5.8 | 1.30E−08 |
| 2: 103035044 | rs1420106 | G | −3.21 | −9.39 | 3.93E−19 |
| 2: 103035889 | rs2293225 | T | −2.42 | −5.89 | 7.97E−09 |
| 2: 103036677 | rs3771156 | T | −2.83 | −7.96 | 1.65E−14 |
| 2: 103038527 | rs3755268 | G | −3.21 | −9.39 | 3.93E−19 |
| 2: 103038587 | rs3755267 | G | −3.15 | −9.15 | 2.57E−18 |
| 2: 103039584 | rs3817465 | T | −3.19 | −9.29 | 8.61E−19 |
| 2: 103039929 | rs2272128 | A | −3.21 | −9.39 | 3.93E−19 |
| 2: 103040945 | rs887972 | A | −2.83 | −8.06 | 8.10E−15 |
| 2: 103041167 | rs887971 | C | −2.84 | −8.02 | 1.12E−14 |
| 2: 103043739 | rs11678975 | A | −2.86 | −8.01 | 1.17E−14 |
| 2: 103045020 | rs11694658 | G | −3.19 | −9.33 | 6.15E−19 |
| 2: 103046880 | rs2160232 | A | −3.18 | −9.35 | 5.26E−19 |
| 2: 103047802 | rs66566526 | T | −2.84 | −8.02 | 1.12E−14 |
| 2: 103048103 | rs6755786 | T | −3.21 | −9.45 | 2.56E−19 |
| 2: 103048157 | rs56331791 | A | −2.84 | −8.02 | 1.12E−14 |
| 2: 103048437 | rs6713618 | T | −3.21 | −9.45 | 2.56E−19 |
| 2: 103048467 | rs6716784 | G | −3.21 | −9.45 | 2.56E−19 |
| 2: 103049910 | rs2041756 | G | −3.21 | −9.41 | 3.30E−19 |
| 2: 103050458 | rs6543134 | G | −3.21 | −9.41 | 3.30E−19 |
| 2: 103050925 | rs2110735 | G | −3.21 | −9.41 | 3.30E−19 |
| 2: 103051144 | rs11681718 | G | −2.84 | −8.02 | 1.12E−14 |
| 2: 103051558 | rs4851582 | C | −2.84 | −8.02 | 1.12E−14 |
| 2: 103052206 | rs2110734 | T | −3.25 | −9.61 | 6.92E−20 |
| 2: 103052995 | rs6746271 | C | −3.21 | −9.41 | 3.30E−19 |
| 2: 103054449 | rs2058660 | A | −3.21 | −9.41 | 3.30E−19 |
| 2: 103054803 | rs2058658 | C | −3.21 | −9.41 | 3.30E−19 |
| 2: 103055420 | rs17027166 | A | −2.86 | −8.01 | 1.16E−14 |
| 2: 103055644 | rs4851009 | A | −3.21 | −9.41 | 3.30E−19 |
| 2: 103055661 | rs55645612 | T | −2.84 | −8.02 | 1.12E−14 |
| 2: 103056096 | rs56166614 | G | −2.84 | −8.02 | 1.12E−14 |
| 2: 103056534 | rs10490204 | C | −2.82 | −7.92 | 2.25E−14 |
| 2: 103057159 | rs17027179 | T | −2.84 | −8.02 | 1.12E−14 |
| 2: 103058328 | rs1558652 | A | −3.21 | −9.41 | 3.30E−19 |
| 2: 103058803 | rs11123927 | T | −2.8 | −7.91 | 2.33E−14 |
| 2: 103059237 | rs10490203 | G | −2.84 | −8.02 | 1.12E−14 |
| 2: 103060024 | rs1558650 | A | −3.14 | −9.13 | 2.94E−18 |
| 2: 103060300 | rs4851583 | C | −2.84 | −8.02 | 1.12E−14 |
| 2: 103060313 | rs4851584 | G | −3.21 | −9.41 | 3.30E−19 |
| 2: 103060851 | rs3771150 | A | −2.84 | −8.08 | 7.01E−15 |
| 2: 103061147 | rs11694360 | A | −2.84 | −8.02 | 1.12E−14 |
| 2: 103061286 | rs11123928 | A | −2.84 | −8.02 | 1.12E−14 |
| 2: 103062116 | rs7597017 | G | −2.84 | −8.02 | 1.12E−14 |
| 2: 103062754 | rs4851585 | A | −2.84 | −8.02 | 1.12E−14 |
| 2: 103062880 | rs6734736 | T | −3.21 | −9.41 | 3.30E−19 |
| 2: 103063369 | rs6708413 | A | −3.21 | −9.36 | 5.00E−19 |
| 2: 103064264 | rs4851586 | C | −3.21 | −9.34 | 5.90E−19 |
| 2: 103064503 | rs11465727 | G | −2.55 | −7.24 | 2.18E−12 |
| 2: 103064511 | rs11465728 | A | −2.55 | −7.24 | 2.18E−12 |
| 2: 103064512 | rs11465729 | T | −2.55 | −7.24 | 2.18E−12 |
| 2: 103065367 | rs56044378 | A | −2.84 | −8.02 | 1.12E−14 |
| 2: 103065908 | rs6543137 | G | −3.21 | −9.41 | 3.30E−19 |
| 2: 103066858 | rs11465730 | G | −2.76 | −7.72 | 8.62E−14 |
| 2: 103067143 | rs11123929 | A | −2.78 | −7.84 | 3.89E−14 |
| 2: 103068787 | rs7559479 | A | −3.16 | −9.26 | 1.08E−18 |
| 2: 103068834 | rs7603250 | A | −3.16 | −9.26 | 1.08E−18 |
| 2: 103070568 | rs917997 | C | −3.16 | −9.26 | 1.08E−18 |
| 2: 103070988 | rs2075185 | G | −3.15 | −9.16 | 2.40E−18 |
| 2: 103073238 | rs11678721 | G | −2.82 | −7.84 | 3.85E−14 |
| 2: 103073293 | rs11677555 | G | −2.76 | −7.72 | 8.62E−14 |
| 2: 103073474 | rs4851587 | T | −3.1 | −9.06 | 5.02E−18 |
| 2: 103073505 | rs4851588 | G | −3.16 | −9.26 | 1.08E−18 |
| 2: 103073950 | rs6728475 | T | −2.7 | −7.54 | 3.06E−13 |
| 2: 103074079 | rs6757276 | G | −3.16 | −9.26 | 1.08E−18 |
| 2: 103074493 | rs4070554 | G | −3.16 | −9.26 | 1.08E−18 |
| 2: 103074651 | rs56117144 | C | −2.81 | −7.92 | 2.17E−14 |
| 2: 103074813 | rs12712150 | C | −2.76 | −7.72 | 8.62E−14 |
| 2: 103075381 | rs57081652 | C | −2.81 | −7.92 | 2.17E−14 |
| 2: 103075561 | rs6761825 | C | −3.16 | −9.26 | 1.08E−18 |
| 2: 103076001 | rs60038017 | C | −2.78 | −7.84 | 3.75E−14 |
| 2: 103076107 | rs10175585 | A | −2.64 | −7.71 | 9.31E−14 |
| 2: 103076151 | rs13394086 | A | −2.44 | −7.59 | 2.12E−13 |
| 2: 103076154 | rs62151860 | A | −2.24 | −6.3 | 7.57E−10 |
| 2: 103076155 | rs62151861 | A | −2.68 | −8.22 | 2.55E−15 |
| 2: 103076210 | rs6705001 | G | −3.14 | −9.21 | 1.59E−18 |
| 2: 103076351 | rs6543141 | A | −3.14 | −9.21 | 1.59E−18 |
| 2: 103076569 | rs6705385 | C | −2.76 | −7.72 | 8.62E−14 |
| 2: 103076670 | rs6705498 | G | −2.76 | −7.72 | 8.62E−14 |
| 2: 103076888 | rs6719196 | T | −2.76 | −7.72 | 8.62E−14 |
| 2: 103077894 | rs7561487 | G | −3.16 | −9.26 | 1.08E−18 |
| 2: 103078740 | rs4241210 | A | −3.16 | −9.26 | 1.08E−18 |
| 2: 103079297 | rs6720564 | C | −3.16 | −9.26 | 1.08E−18 |
| 2: 103079330 | rs17027230 | T | −2.82 | −7.95 | 1.82E−14 |
| 2: 103079619 | rs6717915 | C | −3.16 | −9.26 | 1.08E−18 |
| 2: 103079814 | rs6718157 | T | −3.16 | −9.26 | 1.08E−18 |
| 2: 103080592 | rs2075184 | C | −3.16 | −9.26 | 1.08E−18 |
| 2: 103081695 | rs11676236 | A | −2.76 | −7.72 | 8.62E−14 |
| 2: 103081721 | rs11687013 | A | −2.76 | −7.72 | 8.62E−14 |
| 2: 103082273 | rs917996 | A | −3.16 | −9.26 | 1.08E−18 |
| 2: 103082571 | rs731628 | T | −3.16 | −9.26 | 1.08E−18 |
| 2: 103083453 | rs1403554 | A | −3.13 | −9.14 | 2.86E−18 |
| 2: 103085257 | rs12463588 | G | −2.76 | −7.72 | 8.62E−14 |
| 2: 103085631 | rs10165460 | T | −3.16 | −9.26 | 1.08E−18 |
| 2: 103085660 | rs11123931 | G | −3.16 | −9.26 | 1.08E−18 |
| 2: 103086049 | rs2310302 | C | −2.76 | −7.72 | 8.62E−14 |
| 2: 103086758 | rs12469887 | C | −2.76 | −7.72 | 8.62E−14 |
| 2: 103086770 | rs990171 | C | −3.16 | −9.26 | 1.08E−18 |
| 2: 103087383 | rs56043441 | T | −2.81 | −7.92 | 2.17E−14 |
| 2: 103087652 | rs10195075 | A | −2.74 | −7.65 | 1.40E−13 |
| 2: 103087662 | rs1403553 | G | −2.76 | −7.72 | 8.62E−14 |
| 2: 103088176 | rs4140786 | T | −2.73 | −7.7 | 1.01E−13 |
| 2: 103088517 | rs4140785 | C | −2.78 | −7.85 | 3.54E−14 |
| 2: 103089078 | rs10201184 | C | −2.75 | −7.69 | 1.06E−13 |
| 2: 103089678 | rs4851011 | T | −2.78 | −7.85 | 3.54E−14 |
| 2: 103090127 | rs17027255 | T | −2.78 | −7.82 | 4.46E−14 |
| 2: 103091001 | rs1474309 | T | −2.81 | −7.99 | 1.35E−14 |
| 2: 103091540 | rs17027258 | G | −2.78 | −7.85 | 3.54E−14 |
| 2: 103091715 | rs10197881 | G | −3.16 | −9.23 | 1.44E−18 |
| 2: 103092021 | rs1468791 | G | −3.16 | −9.23 | 1.44E−18 |
| 2: 103092441 | rs1468790 | C | −2.34 | −5.94 | 5.87E−09 |
| 2: 103092503 | rs1468789 | T | −2.21 | −4.97 | 9.95E−07 |
| 2: 103092513 | rs1468788 | T | −2.34 | −5.94 | 5.87E−09 |
| 2: 103092906 | rs7597819 | G | −3.12 | −9.08 | 4.33E−18 |
| 2: 103093081 | rs6737668 | T | −3.16 | −9.11 | 3.48E−18 |
| 2: 103093243 | rs10469840 | C | −3.16 | −9.27 | 1.02E−18 |
| 2: 103094213 | rs759382 | T | −2.94 | −8.45 | 4.81E−16 |
| 2: 103094323 | rs759381 | T | −2.84 | −8.06 | 8.12E−15 |
| 2: 103096695 | rs6543146 | G | −1.73 | −4.65 | 4.52E−06 |
| 2: 103098178 | rs1030026 | C | −1.73 | −4.64 | 4.78E−06 |
| 2: 103098191 | rs1523199 | G | −1.79 | −4.79 | 2.32E−06 |
| 2: 103098474 | rs1523198 | T | −1.79 | −4.79 | 2.32E−06 |
| 2: 103098676 | rs2140316 | A | −1.78 | −4.78 | 2.48E−06 |
| 2: 103100298 | rs74180219 | C | −1.98 | −5.39 | 1.16E−07 |
| 2: 103100300 | rs61604733 | C | −1.97 | −5.35 | 1.46E−07 |
| 2: 103103232 | rs10175045 | C | −1.79 | −4.81 | 2.12E−06 |
| 2: 103103297 | rs10172588 | G | −1.82 | −4.9 | 1.35E−06 |
| 2: 103103879 | rs2310303 | G | −1.72 | −4.65 | 4.38E−06 |
| 2: 103105148 | rs2310304 | C | −1.81 | −4.89 | 1.44E−06 |
| 2: 103106569 | rs6705272 | C | −1.97 | −5.23 | 2.68E−07 |
| 2: 103106773 | rs1523200 | G | −1.91 | −4.57 | 6.44E−06 |
| 2: 103107064 | rs1523201 | T | −1.8 | −4.85 | 1.76E−06 |
| 2: 103108852 | rs7567885 | G | −1.95 | −5.19 | 3.30E−07 |
| 2: 103109676 | rs4851598 | C | −1.95 | −5.19 | 3.30E−07 |
| 2: 103110375 | rs1403548 | T | −1.8 | −4.85 | 1.76E−06 |
| 2: 103111761 | rs12712153 | T | −1.95 | −5.17 | 3.63E−07 |
| 2: 103111920 | rs11687071 | A | −1.95 | −5.19 | 3.30E−07 |
| 2: 103112565 | rs7566063 | A | −1.8 | −4.85 | 1.76E−06 |
| 2: 103112641 | rs7591872 | C | −1.81 | −4.9 | 1.37E−06 |
| 2: 103112658 | rs7591878 | A | −1.85 | −4.98 | 9.51E−07 |
| 2: 103113983 | rs6543150 | T | −1.75 | −4.73 | 3.16E−06 |
| 2: 103114041 | rs6543151 | G | −1.88 | −5.01 | 8.23E−07 |
| 2: 103114043 | rs6543152 | A | −1.88 | −5.01 | 8.23E−07 |
| 2: 103114203 | rs6543153 | C | −1.97 | −5.25 | 2.40E−07 |
| 2: 103114334 | rs6543154 | C | −1.81 | −4.87 | 1.61E−06 |
| 2: 103114895 | rs6543155 | A | −1.81 | −4.89 | 1.44E−06 |
| 2: 103115205 | rs7573566 | C | −1.97 | −5.23 | 2.68E−07 |
| 2: 103115568 | rs11123934 | A | −1.81 | −4.89 | 1.44E−06 |
| 2: 103115838 | rs12987295 | A | −1.98 | −5.25 | 2.42E−07 |
| 2: 103115915 | rs4851012 | T | −1.98 | −5.25 | 2.42E−07 |
| 2: 103116020 | rs4851599 | T | −1.98 | −5.25 | 2.42E−07 |
| 2: 103116135 | rs4851013 | T | −1.94 | −5.2 | 3.18E−07 |
| 2: 103116223 | rs4851600 | G | −1.94 | −5.2 | 3.18E−07 |
| 2: 103116276 | rs4851014 | T | −1.94 | −5.2 | 3.18E−07 |
| 2: 103116305 | rs4851602 | A | −1.85 | −4.97 | 1.01E−06 |
| 2: 103116466 | rs12995030 | G | −1.97 | −5.23 | 2.68E−07 |
| 2: 103117095 | rs11682754 | G | −1.97 | −5.23 | 2.68E−07 |
| 2: 103117268 | rs6728288 | T | −1.97 | −5.23 | 2.68E−07 |
| 2: 103117283 | rs6741235 | A | −1.97 | −5.23 | 2.68E−07 |
| 2: 103117509 | rs56249513 | C | −1.97 | −5.23 | 2.68E−07 |
| 2: 103117618 | rs6741627 | T | −1.97 | −5.23 | 2.68E−07 |
| 2: 103117637 | rs6713906 | G | −1.92 | −5.11 | 4.97E−07 |
| 2: 103117827 | rs6543157 | G | −1.92 | −5.11 | 4.97E−07 |
| 2: 103117910 | rs7591101 | T | −1.89 | −5.01 | 8.17E−07 |
| 2: 103117989 | rs7568122 | T | −1.97 | −5.27 | 2.23E−07 |
| 2: 103118027 | rs2075193 | A | −1.77 | −4.77 | 2.57E−06 |
| 2: 103118128 | rs7593935 | A | −1.97 | −5.24 | 2.50E−07 |
| 2: 103118228 | rs2075192 | G | −1.97 | −5.24 | 2.50E−07 |
| 2: 103118299 | rs2075191 | T | −2 | −5.32 | 1.72E−07 |
| 2: 103118337 | rs2072509 | C | −1.97 | −5.24 | 2.50E−07 |
| 2: 103118342 | rs2072508 | C | −1.83 | −4.95 | 1.07E−06 |
| 2: 103118559 | rs2075190 | T | −1.92 | −5.13 | 4.46E−07 |
| 2: 103118689 | rs2075189 | G | −1.97 | −5.21 | 2.92E−07 |
| 2: 103119029 | rs11690932 | A | −1.97 | −5.23 | 2.68E−07 |
| 2: 103120232 | rs2075188 | A | −1.98 | −5.25 | 2.45E−07 |
| 2: 103120311 | rs2075187 | A | −1.82 | −4.91 | 1.32E−06 |
| 2: 103120356 | rs4851015 | T | −1.82 | −4.91 | 1.32E−06 |
| 2: 103120400 | rs4851016 | T | −1.98 | −5.25 | 2.45E−07 |
| 2: 103120769 | rs4851017 | A | −1.97 | −5.24 | 2.58E−07 |
| 2: 103120868 | rs4851605 | G | −2.03 | −5.42 | 1.01E−07 |
| 2: 103120889 | rs4851606 | A | −1.95 | −5.19 | 3.29E−07 |
| 2: 103123301 | rs13019784 | G | −2.86 | −8.29 | 1.59E−15 |
| 2: 103123633 | rs9989842 | G | −2.88 | −8.39 | 7.89E−16 |
| 2: 103123642 | rs9989749 | A | −2.83 | −8.22 | 2.52E−15 |
| 2: 103123965 | rs6708949 | C | −2.88 | −8.19 | 3.16E−15 |
| 2: 103125018 | rs6724109 | G | −2.86 | −8.29 | 1.59E−15 |
| 2: 103125138 | rs6751949 | A | −2.86 | −8.29 | 1.59E−15 |
| 2: 103125182 | rs6724322 | T | −2.88 | −8.19 | 3.16E−15 |
| 2: 103125457 | rs6739301 | G | −2.86 | −8.29 | 1.59E−15 |
| 2: 103125466 | rs6742381 | C | −2.86 | −8.29 | 1.59E−15 |
| 2: 103125632 | rs4851607 | T | −2.86 | −8.29 | 1.59E−15 |
| 2: 103125736 | rs10195948 | C | −2.86 | −8.29 | 1.59E−15 |
| 2: 103125984 | rs4851608 | T | −2.52 | −7.02 | 9.24E−12 |
| 2: 103127682 | rs10210680 | A | −2.87 | −8.35 | 1.05E−15 |
| 2: 103127963 | rs12712155 | T | −2.9 | −8.29 | 1.55E−15 |
| 2: 103128866 | rs4851609 | C | −2.9 | −8.29 | 1.55E−15 |
| 2: 103129692 | rs11676371 | C | −2.91 | −8.44 | 5.42E−16 |
| 2: 103131679 | rs1476999 | A | −2.49 | −6.87 | 2.32E−11 |
| 2: 103132269 | rs2192758 | G | −2.76 | −7.9 | 2.59E−14 |
| 2: 103132378 | rs2192757 | T | −2.9 | −8.25 | 2.05E−15 |
| 2: 103132640 | rs2216000 | C | −2.52 | −7.02 | 9.24E−12 |
| 2: 103133310 | rs6714379 | G | −2.92 | −8.32 | 1.31E−15 |
| 2: 103134180 | rs1916307 | T | −2.9 | −8.25 | 2.05E−15 |
| 2: 103134652 | rs4851610 | G | −2.9 | −8.25 | 2.05E−15 |
| 2: 103135637 | rs1523204 | A | −2.52 | −7.02 | 9.24E−12 |
| 2: 103135759 | rs1523203 | G | −2.9 | −8.25 | 2.05E−15 |
| 2: 103135938 | rs4851611 | T | −2.88 | −8.16 | 4.09E−15 |
| 2: 103136309 | rs1403550 | C | −2.9 | −8.25 | 2.05E−15 |
| 2: 103136446 | rs1403551 | G | −2.9 | −8.25 | 2.05E−15 |
| 2: 103137015 | rs2058657 | C | −2.91 | −8.31 | 1.40E−15 |
| 2: 103137026 | rs2058656 | G | −2.52 | −7.02 | 9.24E−12 |
| 2: 103137880 | rs4851612 | G | −2.52 | −7.02 | 9.24E−12 |
| 2: 103137990 | rs4851613 | C | −2.9 | −8.25 | 2.05E−15 |
| 2: 103138761 | rs6750851 | G | −2.9 | −8.25 | 2.05E−15 |
| 2: 103138825 | rs6750971 | G | −2.9 | −8.25 | 2.05E−15 |
| 2: 103139298 | rs10193407 | T | −2.83 | −8.06 | 7.88E−15 |
| 2: 103139751 | rs11123935 | G | −2.9 | −8.27 | 1.78E−15 |
| 2: 103140398 | rs4851614 | T | −2.65 | −7.55 | 2.87E−13 |
| 2: 103140472 | rs1357471 | T | −2.68 | −7.63 | 1.64E−13 |
| 2: 103141447 | rs2015478 | G | −2.68 | −7.63 | 1.64E−13 |
| 2: 103141651 | rs1024798 | C | −2.81 | −7.93 | 1.97E−14 |
| 2: 103142491 | rs6712638 | C | −2.81 | −7.93 | 1.97E−14 |
| 2: 103143159 | rs4241211 | G | −2.68 | −7.63 | 1.64E−13 |
| 2: 103144020 | rs12712156 | C | −2.68 | −7.63 | 1.64E−13 |
| 2: 103144242 | rs3849364 | C | −2.68 | −7.63 | 1.64E−13 |
| 2: 103144391 | rs3849365 | A | −2.68 | −7.63 | 1.64E−13 |
| 2: 103144900 | rs3849366 | A | −2.68 | −7.63 | 1.64E−13 |
| 2: 103144944 | rs6739236 | G | −2.68 | −7.63 | 1.64E−13 |
| 2: 103145001 | rs6712696 | A | −2.68 | −7.63 | 1.64E−13 |
| 2: 103145359 | rs1005042 | G | −2.71 | −7.69 | 1.08E−13 |
| 2: 103145426 | rs1005043 | G | −2.68 | −7.63 | 1.64E−13 |
| 2: 103146615 | rs4851018 | T | −2.68 | −7.63 | 1.64E−13 |
| 2: 103146999 | rs4851615 | T | −2.52 | −6.87 | 2.28E−11 |
| 2: 103147256 | rs1003431 | C | −2.68 | −7.63 | 1.64E−13 |
| 2: 103147671 | rs4241212 | G | −2.68 | −7.63 | 1.64E−13 |
| 2: 103148169 | rs741285 | T | −2.68 | −7.63 | 1.64E−13 |
| 2: 103149100 | rs1014286 | A | −2.68 | −7.63 | 1.64E−13 |
| 2: 103149162 | rs2008159 | G | −2.68 | −7.63 | 1.64E−13 |
| 2: 103149182 | rs2008157 | G | −2.68 | −7.64 | 1.50E−13 |
| 2: 103149698 | rs1829849 | C | −2.68 | −7.63 | 1.64E−13 |
| 2: 103150204 | rs10179779 | G | −2.68 | −7.63 | 1.64E−13 |
| 2: 103150306 | rs10179874 | C | −2.47 | −6.71 | 6.53E−11 |
| 2: 103150651 | rs10177737 | C | −2.68 | −7.63 | 1.64E−13 |
| 2: 103150691 | rs12468713 | A | −2.68 | −7.63 | 1.64E−13 |
| 2: 103151109 | rs6737119 | A | −2.68 | −7.63 | 1.64E−13 |
| 2: 103151164 | rs6709284 | G | −2.68 | −7.63 | 1.64E−13 |
| 2: 103151219 | rs6724213 | C | −2.81 | −7.93 | 1.97E−14 |
| 2: 103151319 | rs2177317 | G | −2.68 | −7.63 | 1.64E−13 |
| 2: 103151441 | rs2871474 | A | −2.81 | −7.93 | 1.97E−14 |
| 2: 103151862 | rs4851616 | T | −2.81 | −7.93 | 1.97E−14 |
| 2: 103152060 | rs4851617 | T | −2.68 | −7.63 | 1.64E−13 |
| 2: 103152216 | rs7578425 | G | −2.76 | −7.76 | 6.59E−14 |
| 2: 103152975 | rs10172553 | T | −2.5 | −6.8 | 3.59E−11 |
| 2: 103153780 | rs4292112 | A | −2.65 | −7.55 | 2.87E−13 |
| 2: 103155069 | rs6761291 | T | −2.5 | −6.8 | 3.59E−11 |
| 2: 103156205 | rs7576376 | C | −2.72 | −7.65 | 1.43E−13 |
| 2: 103157352 | rs6543158 | G | −2.81 | −7.93 | 1.97E−14 |
| 2: 103159093 | rs11685483 | C | −2.79 | −7.81 | 4.75E−14 |
| 2: 103160057 | rs6543159 | C | −2.87 | −8.18 | 3.43E−15 |
| 2: 103160443 | rs6739426 | G | −2.81 | −7.93 | 1.97E−14 |
| 2: 103160832 | rs10490202 | G | −2.68 | −7.63 | 1.64E−13 |
| 2: 103161053 | rs11899041 | A | −2.81 | −7.93 | 1.97E−14 |
| 2: 103162861 | rs1811263 | T | −2.5 | −6.8 | 3.59E−11 |
| 2: 103162992 | rs1811262 | C | −2.68 | −7.63 | 1.64E−13 |
| 2: 103165129 | rs12712157 | C | −2.81 | −7.93 | 1.97E−14 |
| 2: 103165504 | rs10194822 | G | −2.53 | −6.87 | 2.33E−11 |
| 2: 103165832 | rs1303960 | A | −2.81 | −7.93 | 1.97E−14 |
| 2: 103166043 | rs2215998 | G | −2.53 | −6.87 | 2.33E−11 |
| 2: 103166298 | rs12712158 | C | −2.59 | −7.02 | 9.00E−12 |
| 2: 103166534 | rs723293 | C | −2.56 | −6.92 | 1.69E−11 |
| 2: 103167724 | rs7581853 | T | −2.69 | −7.67 | 1.27E−13 |
| 2: 103168605 | rs6753722 | A | −2.71 | −7.73 | 8.37E−14 |
| 2: 103168977 | rs10193009 | T | −2.69 | −7.73 | 8.21E−14 |
| 2: 103169682 | rs11123937 | A | −2.69 | −7.65 | 1.43E−13 |
| 2: 103169982 | rs12712159 | A | −2.69 | −7.71 | 9.08E−14 |
| 2: 103170181 | rs10196579 | T | −2.5 | −6.8 | 3.59E−11 |
| 2: 103170919 | rs997056 | G | −2.68 | −7.63 | 1.64E−13 |
| 2: 103171066 | rs2310295 | A | −2.5 | −6.8 | 3.59E−11 |
| 2: 103172263 | rs2110737 | A | −2.69 | −7.67 | 1.27E−13 |
| 2: 103173059 | rs2005881 | A | −2.81 | −7.88 | 2.89E−14 |
| 2: 103174935 | rs1989184 | G | −2.81 | −7.93 | 1.97E−14 |
| 2: 103175376 | rs10171438 | A | −2.53 | −6.87 | 2.33E−11 |
| 2: 103175749 | rs10210658 | G | −2.7 | −7.71 | 9.21E−14 |
| 2: 103176411 | rs4851619 | T | −2.81 | −7.93 | 1.97E−14 |
| 2: 103176797 | rs6761871 | C | −2.74 | −7.76 | 6.65E−14 |
| 2: 103177414 | rs10202404 | T | −2.53 | −6.87 | 2.33E−11 |
| 2: 103177565 | rs4851019 | A | −2.5 | −6.81 | 3.41E−11 |
| 2: 103182273 | rs12469973 | G | −2.45 | −6.68 | 7.63E−11 |
| 2: 103188785 | rs11123938 | G | −2.34 | −6.34 | 5.93E−10 |
| 2: 103208610 | rs1861229 | G | −2.02 | −5.45 | 8.66E−08 |
| 2: 103273676 | rs11677922 | A | −1.67 | −4.69 | 3.74E−06 |
| 2: 103287966 | rs12052753 | C | 1.86 | 4.95 | 1.09E−06 |
| 2: 137575582 | rs199977663 | A | 2.7 | 4.99 | 8.87E−07 |
| 2: 16200946 | rs7605722 | A | −1.67 | −4.61 | 5.36E−06 |
| 2: 16201132 | rs7605772 | A | −1.67 | −4.61 | 5.36E−06 |
| 2: 16202446 | rs13427501 | G | −1.67 | −4.61 | 5.36E−06 |
| 2: 16204606 | rs7600440 | A | −1.72 | −4.79 | 2.33E−06 |
| 2: 16206193 | rs4499395 | T | −1.72 | −4.8 | 2.22E−06 |
| 2: 16207141 | rs6760942 | A | −1.72 | −4.8 | 2.22E−06 |
| 2: 16212065 | rs28432381 | A | 1.68 | 4.64 | 4.65E−06 |
| 2: 16214286 | rs4073262 | A | 1.62 | 4.51 | 8.34E−06 |
| 20: 51381201 | rs111246464 | T | 3.45 | 4.59 | 5.76E−06 |
| 20: 55270493 | rs4583526 | G | −1.69 | −4.5 | 8.78E−06 |
| 4: 11286652 | rs2141304 | G | 2.12 | 4.65 | 4.47E−06 |
| 4: 11291091 | rs10805282 | G | −2.1 | −4.56 | 6.72E−06 |
| 4: 11291169 | rs10805283 | G | −2.1 | −4.56 | 6.72E−06 |
| 4: 11291539 | rs13128031 | T | −2.1 | −4.56 | 6.72E−06 |
| 4: 96370605 | rs4103380 | C | 1.8 | 4.59 | 5.76E−06 |
| 5: 156401928 | rs191531802 | G | 2.74 | 4.95 | 1.10E−06 |
| 5: 156450845 | rs117979984 | C | 3.54 | 4.62 | 5.21E−06 |
| 5: 156457158 | rs138665140 | G | 3.54 | 4.62 | 5.21E−06 |
| 5: 35237736 | rs7705676 | C | 2.21 | 4.55 | 7.06E−06 |
| 5: 35238047 | rs7727503 | G | 2.17 | 4.52 | 8.11E−06 |
| 5: 35238245 | rs7710279 | C | 2.21 | 4.55 | 7.06E−06 |
| 5: 35238365 | rs10472952 | C | 2.21 | 4.55 | 7.06E−06 |
| 5: 35238779 | rs9292581 | A | 2.21 | 4.55 | 7.06E−06 |
| 5: 35239028 | rs4703514 | C | 2.19 | 4.56 | 6.61E−06 |
| 5: 35239203 | rs13361375 | G | 2.21 | 4.55 | 7.06E−06 |
| 6: 146685324 | rs2300619 | C | 2.2 | 4.61 | 5.48E−06 |
| 7: 106147658 | rs17288506 | C | 2.54 | 4.55 | 6.91E−06 |
| 7: 106150256 | rs13229385 | G | 2.5 | 4.48 | 9.57E−06 |
| 7: 106156253 | rs9886235 | C | 2.5 | 4.58 | 6.28E−06 |
| 7: 106160902 | rs13238247 | C | 2.5 | 4.58 | 6.28E−06 |
| 7: 106162503 | rs10279909 | G | 2.5 | 4.58 | 6.28E−06 |
| 7: 106162983 | rs17837506 | G | 2.5 | 4.58 | 6.28E−06 |
| 7: 106163550 | rs13222141 | A | 2.5 | 4.58 | 6.28E−06 |
| 7: 81588636 | rs1229502 | A | 2.04 | 4.66 | 4.36E−06 |
| 8: 21616063 | rs6587006 | C | −1.93 | −5.43 | 9.68E−08 |
| 9: 111538148 | rs5006678 | C | −2.51 | −4.86 | 1.65E−06 |
| 9: 111538149 | rs5006679 | C | −2.19 | −4.64 | 4.77E−06 |
| 9: 131696704 | rs7849649 | A | 2.06 | 4.53 | 7.84E−06 |
| 9: 71876805 | rs7047059 | T | 4.25 | 5.65 | 3.05E−08 |
| 9: 71883053 | rs10283781 | G | 4.13 | 5.49 | 6.84E−08 |
| 9: 71903717 | rs10867298 | T | 4.19 | 5.53 | 5.81E−08 |
| 9: 71903721 | rs10867299 | C | 4.19 | 5.53 | 5.81E−08 |
| 9: 71903965 | rs57491316 | T | 4.19 | 5.53 | 5.81E−08 |
| 9: 71904249 | rs56795581 | A | 4.19 | 5.53 | 5.81E−08 |
| 9: 71905304 | rs11137952 | A | 4.19 | 5.53 | 5.81E−08 |
| 9: 71905591 | rs201118747 | G | 4.13 | 5.49 | 6.84E−08 |
| 9: 71905605 | rs199851061 | C | 4.13 | 5.49 | 6.84E−08 |
| 9: 71906042 | rs138313346 | T | 4.13 | 5.49 | 6.84E−08 |
| 9: 71906974 | rs11137959 | G | 4.13 | 5.49 | 6.84E−08 |
| X: 166155 | rs56242654 | T | −1.58 | −4.49 | 9.37E−06 |

TABLE 5

| | | Effective | | | Causal |
|---|---|---|---|---|---|
| Position | SNP | Allele | β (SE) | P-value | probability^{a} |
| chr2: 102966067 | rs1921622 | A | −3.346 (0.327) | 5.35E−22 | 0.9999 |
| chr2: 102960584 | rs12712142 | A | −2.919 (0.344) | 4.41E−16 | 0.1745 |
| chr2: 102964742 | rs13017455 | T | −2.919 (0.344) | 4.41E−16 | 0.1745 |
| chr2: 102967844 | rs11123923 | A | −2.919 (0.344) | 4.41E−16 | 0.1745 |
| chr2: 102931826 | rs13020553 | G | −2.907 (0.344) | 4.75E−16 | 0.0556 |
| chr2: 102932562 | rs950880 | A | −2.907 (0.344) | 4.75E−16 | 0.0556 |
| chr2: 102939036 | rs13001325 | T | −2.890 (0.344) | 7.72E−16 | 0.0534 |
| chr2: 102948470 | rs1420104 | A | −2.890 (0.344) | 7.72E−16 | 0.0534 |
| chr2: 102949161 | rs12479210 | T | −2.890 (0.344) | 7.72E−16 | 0.0534 |
| chr2: 102960485 | rs13001714 | G | −2.890 (0.344) | 7.72E−16 | 0.0534 |
| chr2: 102963072 | rs6543119 | T | −2.904 (0.344) | 5.91E−16 | 0.0508 |
| chr2: 102957716 | rs1420101 | T | −2.895 (0.344) | 6.87E−16 | 0.0253 |
| chr2: 102950822 | rs13019081 | C | −2.905 (0.346) | 8.44E−16 | 0.0250 |
| chr2: 102926362 | rs12470864 | A | −2.880 (0.343) | 7.15E−16 | 0.0168 |
| chr2: 102969606 | rs35224028 | C | −2.878 (0.342) | 5.99E−16 | 0.0154 |
| chr2: 102974417 | rs12998521 | T | −2.877 (0.346) | 1.26E−15 | 0.0044 |
| chr2: 102974129 | rs12999364 | T | −2.858 (0.347) | 2.30E−15 | 0.0022 |
| chr2: 102975336 | rs12987977 | G | −2.858 (0.347) | 2.30E−15 | 0.0022 |
| chr2: 102979028 | rs2287037 | T | −2.858 (0.347) | 2.30E−15 | 0.0022 |
| chr2: 102983247 | rs4851569 | A | −2.858 (0.347) | 2.30E−15 | 0.0022 |
| chr2: 102984671 | rs1882348 | A | −2.858 (0.347) | 2.30E−15 | 0.0022 |
| chr2: 102984279 | rs1420098 | C | −2.854 (0.346) | 2.12E−15 | 0.0019 |

TABLE 6

| | Chinese_cohort_2 (WGS) | | | |
|---|---|---|---|---|
| | Healthy control | | Alzheimer's disease | |
| n | 829 | | 867 | |
| Age, years (SD) | 69.14 | (7.12) | 71.15 | (7.26) |
| Sex, male (%) | 394 | (47.53%) | 391 | (45.10%) |
| MMSE score (SD) | 28.60 | (1.41) | 14.80 | (6.27) |
| APOE-ε4, allele frequency | 9.41% | | 26.07% | |
| APOE-ε2, allele frequency | 8.20% | | 4.84% | |
| | Chinese_cohort_2 (Array) | | | |
| | Healthy control | | Alzheimer's disease | |
| n | 311 | | 425 | |
| Age, years (SD) | 69.82 | (6.42) | 71.17 | (7.13) |
| Sex, male (%) | 119 | (38.26%) | 169 | (39.76%) |
| MMSE score (SD) | 28.27 | (1.77) | 15.06 | (6.08) |
| APOE-ε4, allele frequency | 8.52% | | 32.35% | |
| APOE-ε2, allele frequency | 7.88% | | 5.29% | |
| | LOAD | | | |
| | Healthy control | | Alzheimer's disease | |
| n | 2,231 | | 464 | |
| Age, years (SD) | 80.68 | (10.75) | 83.80 | (6.62) |
| Sex, male (%) | 871 | (39.04%) | 144 | (31.03%) |
| APOE-ε4, allele frequency | 20.77% | | 46.34% | |
| APOE-ε2, allele frequency | 6.90% | | 2.80% | |
| | ADC | | | |
| | Healthy control | | Alzheimer's disease | |
| n | 1,430 | | 3,422 | |
| Age, years (SD) | 76.70 | (8.36) | 79.82 | (7.64) |
| Sex, male (%) | 902 | (63.08%) | 1809 | (52.86%) |
| APOE-ε4, allele frequency | 13.39% | | 39.04% | |
| APOE-ε2, allele frequency | 9.02% | | 3.34% | |
| | ADNI | | | |
| | Healthy control | | Alzheimer's disease | |
| n | 290 | | 378 | |
| Age, years (SD) | 78.51 | (6.64) | 78.59 | (6.95) |
| Sex, male (%) | 147 | (50.68%) | 231 | (61.11%) |

| | | |
|---|---|---|
| APOE-ε4, allele frequency | 13.45% | 42.33% |
| APOE-ε2, allele frequency | 7.24% | 2.92% |

TABLE 7

| | | Overall | | | | | APOE-ε4 carriers | | |
|---|---|---|---|---|---|---|---|---|---|
| | | N | HC | AD | OR | P | n | HC | AD |
| rs1921622 | Chinese_cohort_1 | 690 | 49.13% | 51.01% | 0.84 | 0.20 | 171 | 57.00% | 50.41% |
| A allele | Chinese_cohort_2 | 1,696 | 42.58% | 43.02% | 1.01 | 0.82 | 517 | 44.44% | 42.76% |
| | (WGS) | | | | | | | | |
| | Chinese_cohort_2 | 736 | 43.41% | 42.94% | 0.98 | 0.86 | 273 | 52.88% | 41.40% |
| | (Array) | | | | | | | | |
| | LOAD | 2,695 | 52.84% | 50.43% | 0.84 | 0.01 | 1,194 | 54.78% | 52.30% |
| | ADC | 4,852 | 53.57% | 52.70% | 0.96 | 0.44 | 2,521 | 53.76% | 52.84% |
| | ADNI | 668 | 53.45% | 53.31% | 0.94 | 0.62 | 324 | 52.82% | 54.15% |

| | | APOE-ε4 carriers | | APOE-ε4 noncarriers | | | | |
|---|---|---|---|---|---|---|---|---|
| | | OR | P | n | HC | AD | OR | P |
| rs1921622 | Chinese_cohort_1 | 0.50 | 0.01 | 519 | 47.80% | 51.33% | 1.01 | 0.92 |
| A allele | Chinese_cohort_2 | 0.93 | 0.60 | 1179 | 42.19% | 43.22% | 1.03 | 0.64 |
| | (WGS) | | | | | | | |
| | Chinese_cohort_2 | 0.61 | 0.02 | 463 | 41.51% | 44.60% | 1.15 | 0.31 |
| | (Array) | | | | | | | |
| | LOAD | 0.84 | 0.06 | 1,501 | 51.66% | 44.87% | 0.70 | 0.01 |
| | ADC | 0.97 | 0.79 | 2,331 | 53.50% | 52.46% | 0.96 | 0.52 |
| | ADNI | 1.02 | 0.90 | 344 | 53.65% | 51.60% | 0.90 | 0.55 |

TABLE 8

| | | Male APOE-ε4 carriers | | | | | Female APOE-ε4 carriers | | | | |
|---|---|---|---|---|---|---|---|---|---|---|---|
| | | n | HC | AD | OR | P | n | HC | AD | OR | P |
| rs1921622 | Chinese_cohort_1 | 62 | 54.00% | 60.81% | 0.42 | 0.14 | 109 | 60.00% | 45.83% | 0.45 | 0.02 |
| A allele | Chinese_cohort_2 | 225 | 35.91% | 45.13% | 1.44 | 0.07 | 292 | 52.74% | 41.09% | 0.63 | 0.01 |
| | (WGS) | | | | | | | | | | |
| | Chinese_cohort_2 | 91 | 46.15% | 44.87% | 1.02 | 0.95 | 182 | 55.13% | 39.51% | 0.51 | 0.01 |
| | (Array) | | | | | | | | | | |
| | LOAD | 456 | 53.35% | 53.98% | 0.95 | 0.74 | 738 | 55.75% | 51.49% | 0.78 | 0.03 |
| | ADC | 1,349 | 53.71% | 51.65% | 0.99 | 0.95 | 1,172 | 53.84% | 54.12% | 0.97 | 0.84 |
| | ADNI | 194 | 48.72% | 56.77% | 1.39 | 0.23 | 130 | 57.81% | 50.00% | 0.68 | 0.20 |

TABLE 9

| ASO | sense | antisense |
|---|---|---|
| hsST2-1 | GGTTCCAAGAGATCCATCAA | TTGATGGATCTCTTGGAACC |
| hsST2-2 | AGAGATCCATCAAGACAATG | CATTGTCTTGATGGATCTCT |
| hsST2-3 | GAGATCCATCAAGACAATGG | CCATTGTCTTGATGGATCTC |
| hsST2-4 | TTCTTCGGGATGTTGTTTGC | GCAAACAACATCCCGAAGAA |
| hsST2-5 | TCTTCGGGATGTTGTTTGCT | AGCAAACAACATCCCGAAGA |
| hsST2-6 | GAGCTTCTCTGCTGCTTAAA | TTTAAGCAGCAGAGAAGCTC |
| hsST2-7 | GCTGCTTAAATTGTTCGTCC | GGACGAACAATTTAAGCAGC |
| hsST2-8 | GCTTAAATTGTTCGTCCTCC | GGAGGACGAACAATTTAAGC |
| hsST2-9 | CCTCCTATCGTTGGTTTGTC | GACAAACCAACGATAGGAGG |
| hsST2-10 | TCCTATCGTTGGTTTGTCTA | TAGACAAACCAACGATAGGA |
| hsST2-11 | CCTATCGTTGGTTTGTCTAG | CTAGACAAACCAACGATAGG |
| hsST2-12 | TATCGTTGGTTTGTCTAGAA | TTCTAGACAAACCAACGATA |
| hsST2-13 | ATCGTTGGTTTGTCTAGAAC | GTTCTAGACAAACCAACGAT |
| hsST2-14 | TCGTTGGTTTGTCTAGAACA | TGTTCTAGACAAACCAACGA |
| hsST2-15 | CGTTGGTTTGTCTAGAACAC | GTGTTCTAGACAAACCAACG |
| hsST2-16 | TAGAACACTCAGCTGCTTCT | AGAAGCAGCTGAGTGTTCTA |
| hsST2-17 | TCTAACTTTATGAACTCCCT | AGGGAGTTCATAAAGTTAGA |
| hsST2-18 | AGGAAATGCACCAACAACCG | CGGTTGTTGGTGCATTTCCT |
| hsST2-19 | GGAAATGCACCAACAACCGT | ACGGTTGTTGGTGCATTTCC |
| hsST2-20 | AAATGCACCAACAACCGTAA | TTACGGTTGTTGGTGCATTT |
| hsST2-21 | TGCACCAACAACCGTAAACT | AGTTTACGGTTGTTGGTGCA |
| hsST2-22 | GCACCAACAACCGTAAACTG | CAGTTTACGGTTGTTGGTGC |
| hsST2-23 | CACCAACAACCGTAAACTGA | TCAGTTTACGGTTGTTGGTG |
| hsST2-24 | ACCAACAACCGTAAACTGAA | TTCAGTTTACGGTTGTTGGT |
| hsST2-25 | CCAACAACCGTAAACTGAAC | GTTCAGTTTACGGTTGTTGG |
| hsST2-26 | ACAACCGTAAACTGAACGTG | CACGTTCAGTTTACGGTTGT |
| hsST2-27 | CAACCGTAAACTGAACGTGT | ACACGTTCAGTTTACGGTTG |
| hsST2-28 | AACCGTAAACTGAACGTGTT | AACACGTTCAGTTTACGGTT |
| hsST2-29 | ACCGTAAACTGAACGTGTTC | GAACACGTTCAGTTTACGGT |
| hsST2-30 | CCGTAAACTGAACGTGTTCT | AGAACACGTTCAGTTTACGG |
| hsST2-31 | CGTAAACTGAACGTGTTCTT | AAGAACACGTTCAGTTTACG |
| hsST2-32 | CATGTTGTAAGCATGGTCCG | CGGACCATGCTTACAACATG |
| hsST2-33 | AAGCATGGTCCGTTCTATAC | GTATAGAACGGACCATGCTT |
| hsST2-34 | AGCATGGTCCGTTCTATACC | GGTATAGAACGGACCATGCT |
| hsST2-35 | GCATGGTCCGTTCTATACCT | AGGTATAGAACGGACCATGC |
| hsST2-36 | CATGGTCCGTTCTATACCTT | AAGGTATAGAACGGACCATG |
| hsST2-37 | ATGGTCCGTTCTATACCTTT | AAAGGTATAGAACGGACCAT |
| hsST2-38 | TGGTCCGTTCTATACCTTTT | AAAAGGTATAGAACGGACCA |
| hsST2-39 | CGTTCTATACCTTTTTCTGG | CCAGAAAAAGGTATAGAACG |
| hsST2-40 | ACACTCATTTTGTTAGCGAG | CTCGCTAACAAAATGAGTGT |
| hsST2-41 | CACTCATTTTGTTAGCGAGG | CCTCGCTAACAAAATGAGTG |
| hsST2-42 | AGGGGAAGTATCAAACTACT | AGTAGTTTGATACTTCCCCT |
| hsST2-43 | GAGAAAATCCTAGGTGCTAC | GTAGCACCTAGGATTTTCTC |
| hsST2-44 | ACCATACCACACACATTATC | GATAATGTGTGTGGTATGGT |
| hsST2-45 | AAGGTACATGGAACACACGG | CCGTGTGTTCCATGTACCTT |
| hsST2-46 | CACACGGGAAGTCTGGTAGC | GCTACCAGACTTCCCGTGTG |
| hsST2-47 | ACACGGGAAGTCTGGTAGCT | AGCTACCAGACTTCCCGTGT |
| hsST2-48 | CACGGGAAGTCTGGTAGCTC | GAGCTACCAGACTTCCCGTG |
| hsST2-49 | ACGGGAAGTCTGGTAGCTCA | TGAGCTACCAGACTTCCCGT |
| hsST2-50 | CGGGAAGTCTGGTAGCTCAG | CTGAGCTACCAGACTTCCCG |
| hsST2-51 | GAAGTCTGGTAGCTCAGCCC | GGGCTGAGCTACCAGACTTC |
| hsST2-52 | TCACCATGTAATTCAGGTAC | GTACCTGAATTACATGGTGA |
| hsST2-53 | CACCATGTAATTCAGGTACC | GGTACCTGAATTACATGGTG |
| hsST2-54 | TGTAATTCAGGTACCACGTA | TACGTGGTACCTGAATTACA |
| hsST2-55 | TAATTCAGGTACCACGTATT | AATACGTGGTACCTGAATTA |
| hsST2-56 | AATTCAGGTACCACGTATTC | GAATACGTGGTACCTGAATT |
| hsST2-57 | ATTCAGGTACCACGTATTCC | GGAATACGTGGTACCTGAAT |
| hsST2-58 | CAGGTACCACGTATTCCAGG | CCTGGAATACGTGGTACCTG |
| hsST2-59 | AGGTACCACGTATTCCAGGG | CCCTGGAATACGTGGTACCT |
| hsST2-60 | GGTACCACGTATTCCAGGGA | TCCCTGGAATACGTGGTACC |
| hsST2-61 | TACCACGTATTCCAGGGAGC | GCTCCCTGGAATACGTGGTA |
| hsST2-62 | ACCACGTATTCCAGGGAGCC | GGCTCCCTGGAATACGTGGT |
| hsST2-63 | CCACGTATTCCAGGGAGCCT | AGGCTCCCTGGAATACGTGG |
| hsST2-64 | ACGTATTCCAGGGAGCCTTT | AAAGGCTCCCTGGAATACGT |
| hsST2-65 | GCCCTCAGTTTGCAGTATAC | GTATACTGCAAACTGAGGGC |
| hsST2-66 | GCAGTATACACACTTTCCAA | TTGGAAAGTGTGTATACTGC |
| hsST2-67 | GGTCACATTGCCTTACCTAA | TTAGGTAAGGCAATGTGACC |
| hsST2-68 | GTCACATTGCCTTACCTAAA | TTTAGGTAAGGCAATGTGAC |
| hsST2-69 | TTGACAGTCTGCTCAACACG | CGTGTTGAGCAGACTGTCAA |
| hsST2-70 | TGACAGTCTGCTCAACACGA | TCGTGTTGAGCAGACTGTCA |
| hsST2-71 | GACAGTCTGCTCAACACGAC | GTCGTGTTGAGCAGACTGTC |
| hsST2-72 | ACAGTCTGCTCAACACGACT | AGTCGTGTTGAGCAGACTGT |
| hsST2-73 | CAGTCTGCTCAACACGACTG | CAGTCGTGTTGAGCAGACTG |
| hsST2-74 | AGTCTGCTCAACACGACTGC | GCAGTCGTGTTGAGCAGACT |
| hsST2-75 | TCTGCTCAACACGACTGCAA | TTGCAGTCGTGTTGAGCAGA |
| hsST2-76 | TGCTCAACACGACTGCAAGC | GCTTGCAGTCGTGTTGAGCA |
| hsST2-77 | GCTCAACACGACTGCAAGCT | AGCTTGCAGTCGTGTTGAGC |
| hsST2-78 | ATCTTTGGGTCCTTAGTGCA | TGCACTAAGGACCCAAAGAT |
| hsST2-79 | CTTTGGGTCCTTAGTGCAAT | ATTGCACTAAGGACCCAAAG |
| hsST2-80 | TTGGGTCCTTAGTGCAATAC | GTATTGCACTAAGGACCCAA |
| hsST2-81 | TTAGTGCAATACCTGGCAGC | GCTGCCAGGTATTGCACTAA |
| hsST2-82 | CTTCTCTAGCTATAAGTCTT | AAGACTTATAGCTAGAGAAG |
| hsST2-83 | GCTATAAGTCTTAATTATAC | GTATAATTAAGACTTATAGC |
| hsST2-84 | TTGGTGGCATCACAAATAGC | GCTATTTGTGATGCCACCAA |
| hsST2-85 | TGGTGGCATCACAAATAGCC | GGCTATTTGTGATGCCACCA |
| hsST2-86 | TATCATCACTATGCCCAGAC | GTCTGGGCATAGTGATGATA |
| hsST2-87 | ATCATCACTATGCCCAGACA | TGTCTGGGCATAGTGATGAT |
| hsST2-88 | GTCTCTGAATGCAATGTTGT | ACAACATTGCATTCAGAGAC |
| hsST2-89 | GACATCCTGTTTGCTATATG | CATATAGCAAACAGGATGTC |
| hsST2-90 | TGAAGCATTGTATCCCGTAT | ATACGGGATACAATGCTTCA |
| hsST2-91 | GAAGCATTGTATCCCGTATA | TATACGGGATACAATGCTTC |
| hsST2-92 | AAGCATTGTATCCCGTATAA | TTATACGGGATACAATGCTT |
| hsST2-93 | AGCATTGTATCCCGTATAAA | TTTATACGGGATACAATGCT |
| hsST2-94 | GCATTGTATCCCGTATAAAA | TTTTATACGGGATACAATGC |
| hsST2-95 | CATTGTATCCCGTATAAAAG | CTTTTATACGGGATACAATG |
| hsST2-96 | ATTGTATCCCGTATAAAAGG | CCTTTTATACGGGATACAAT |
| hsST2-97 | GTATCCCGTATAAAAGGAAG | CTTCCTTTTATACGGGATAC |
| hsST2-98 | TACACTCATCACTCCTCAGG | CCTGAGGAGTGATGAGTGTA |
| hsST2-99 | AATGTGCCTGTCAAATAGCC | GGCTATTTGACAGGCACATT |
| hsST2-100 | ATGTGCCTGTCAAATAGCCA | TGGCTATTTGACAGGCACAT |
| hsST2-101 | AGCCAAAGAGTGTTAAACCC | GGGTTTAACACTCTTTGGCT |
| hsST2-102 | GTGGTATGGTTAGGATTCAT | ATGAATCCTAACCATACCAC |
| hsST2-103 | GTATGGTTAGGATTCATCCA | TGGATGAATCCTAACCATAC |
| hsST2-104 | AATATGGTGTAATCGTGGCA | TGCCACGATTACACCATATT |
| hsST2-105 | ATATGGTGTAATCGTGGCAG | CTGCCACGATTACACCATAT |
| hsST2-106 | TATGGTGTAATCGTGGCAGG | CCTGCCACGATTACACCATA |
| hsST2-107 | TGGTGTAATCGTGGCAGGTC | GACCTGCCACGATTACACCA |
| hsST2-108 | GGTGTAATCGTGGCAGGTCT | AGACCTGCCACGATTACACC |
| hsST2-109 | TGTAATCGTGGCAGGTCTCT | AGAGACCTGCCACGATTACA |
| hsST2-110 | GTAATCGTGGCAGGTCTCTG | CAGAGACCTGCCACGATTAC |
| hsST2-111 | TAATCGTGGCAGGTCTCTGC | GCAGAGACCTGCCACGATTA |
| hsST2-112 | AATCGTGGCAGGTCTCTGCC | GGCAGAGACCTGCCACGATT |
| hsST2-113 | TCATAACTCATGTAGATGGC | GCCATCTACATGAGTTATGA |
| hsST2-114 | TAACTCATGTAGATGGCTAT | ATAGCCATCTACATGAGTTA |
| hsST2-115 | CTCATGTAGATGGCTATAAG | CTTATAGCCATCTACATGAG |
| hsST2-116 | TCATGTAGATGGCTATAAGT | ACTTATAGCCATCTACATGA |
| hsST2-117 | CATGTAGATGGCTATAAGTG | CACTTATAGCCATCTACATG |
| hsST2-118 | GTAGATGGCTATAAGTGCCG | CGGCACTTATAGCCATCTAC |
| hsST2-119 | TAGATGGCTATAAGTGCCGT | ACGGCACTTATAGCCATCTA |
| hsST2-120 | AGATGGCTATAAGTGCCGTA | TACGGCACTTATAGCCATCT |
| hsST2-121 | GATGGCTATAAGTGCCGTAG | CTACGGCACTTATAGCCATC |
| hsST2-122 | ATGGCTATAAGTGCCGTAGT | ACTACGGCACTTATAGCCAT |
| hsST2-123 | TGGCTATAAGTGCCGTAGTG | CACTACGGCACTTATAGCCA |
| hsST2-124 | CTATAAGTGCCGTAGTGTTC | GAACACTACGGCACTTATAG |
| hsST2-125 | TATAAGTGCCGTAGTGTTCT | AGAACACTACGGCACTTATA |
| hsST2-126 | GTGCCGTAGTGTTCTGTGGG | CCCACAGAACACTACGGCAC |
| hsST2-127 | CAGTGATAAGTGTGGCACCC | GGGTGCCACACTTATCACTG |
| hsST2-128 | ATCAAGGTGCTATGTGAGGG | CCCTCACATAGCACCTTGAT |
| hsST2-129 | CTATGTGAGGGCCATGCTTG | CAAGCATGGCCCTCACATAG |
| hsST2-130 | ATGTGAGGGCCATGCTTGGG | CCCAAGCATGGCCCTCACAT |
| hsST2-131 | CCCAGTAGACCCTGCAGCCA | TGGCTGCAGGGTCTACTGGG |
| hsST2-132 | GTGTACTCCTGCTTTGCTAC | GTAGCAAAGCAGGAGTACAC |
| hsST2-133 | TGTACTCCTGCTTTGCTACG | CGTAGCAAAGCAGGAGTACA |
| hsST2-134 | TCCTGCTTTGCTACGTTATC | GATAACGTAGCAAAGCAGGA |
| hsST2-135 | TTTGCTACGTTATCATTGAT | ATCAATGATAACGTAGCAAA |
| hsST2-136 | TTGATCAATGTCCCTGGTTA | TAACCAGGGACATTGATCAA |
| hsST2-137 | GATCAATGTCCCTGGTTACC | GGTAACCAGGGACATTGATC |
| hsST2-138 | TGGTTACACTCTGTTTCAGC | GCTGAAACAGAGTGTAACCA |
| hsST2-139 | ATGGGAAGCATGGCGGCATC | GATGCCGCCATGCTTCCCAT |
| hsST2-140 | CCACCTCGAATACTGGGGAT | ATCCCCAGTATTCGAGGTGG |
| hsST2-141 | GCAATACCTGGCAGCTAGCC | GGCTAGCTGCCAGGTATTGC |
| hsST2-142 | AATAGCCAAAGCAGGGTGGA | TCCACCCTGCTTTGGCTATT |
| hsST2-143 | ACAGAGGTGGACTGATCCTT | AAGGATCAGTCCACCTCTGT |
| hsST2-144 | GTATAAAAGGAAGGAAAGAG | CTCTTTCCTTCCTTTTATAC |
| msST2-1 | GTCCCTCACACATTGCTTGA | TCAAGCAATGTGTGAGGGAC |
| msST2-2 | TGCTTGAATAAATTGGCTGA | TCAGCCAATTTATTCAAGCA |
| msST2-3 | ATCAGCTGTGCACTGCATCC | GGATGCAGTGCACAGCTGAT |
| msST2-4 | GGAATAGTTGGCCTGTTTCA | TGAAACAGGCCAACTATTCC |
| msST2-5 | GTTGGCCTGTTTCATCAAGT | ACTTGATGAAACAGGCCAAC |
| msST2-6 | CATCAAGTGTTCTTCTCACG | CGTGAGAAGAACACTTGATG |
| msST2-7 | CAGGAATCTCTGTCATTGTA | TACAATGACAGAGATTCCTG |
| msST2-8 | CCCTTAATGTGTTCTTGACC | GGTCAAGAACACATTAAGGG |
| msST2-9 | CCTTAATGTGTTCTTGACCC | GGGTCAAGAACACATTAAGG |
| msST2-10 | CACCCAATAGTGGCTTTTGC | GCAAAAGCCACTATTGGGTG |
| msST2-11 | ACCCAATAGTGGCTTTTGCA | TGCAAAAGCCACTATTGGGT |
| msST2-12 | CCCAATAGTGGCTTTTGCAG | CTGCAAAAGCCACTATTGGG |
| msST2-13 | CAATAGTGGCTTTTGCAGTC | GACTGCAAAAGCCACTATTG |
| msST2-14 | CAGTCATCCTTTGTACCGAC | GTCGGTACAAAGGATGACTG |
| msST2-15 | AGTCATCCTTTGTACCGACT | AGTCGGTACAAAGGATGACT |
| msST2-16 | GTCATCCTTTGTACCGACTA | TAGTCGGTACAAAGGATGAC |
| msST2-17 | TCATCCTTTGTACCGACTAC | GTAGTCGGTACAAAGGATGA |
| msST2-18 | ATCCTTTGTACCGACTACAA | TTGTAGTCGGTACAAAGGAT |
| msST2-19 | TCCTTTGTACCGACTACAAG | CTTGTAGTCGGTACAAAGGA |
| msST2-20 | CCTTTGTACCGACTACAAGG | CCTTGTAGTCGGTACAAAGG |
| msST2-21 | CTTTGTACCGACTACAAGGG | CCCTTGTAGTCGGTACAAAG |
| msST2-22 | TTGTACCGACTACAAGGGAC | GTCCCTTGTAGTCGGTACAA |
| msST2-23 | ACCGACTACAAGGGACATTG | CAATGTCCCTTGTAGTCGGT |
| msST2-24 | CCGACTACAAGGGACATTGG | CCAATGTCCCTTGTAGTCGG |
| msST2-25 | CGACTACAAGGGACATTGGT | ACCAATGTCCCTTGTAGTCG |
| msST2-26 | TTGGTATTGGTAGTGGGTTC | GAACCCACTACCAATACCAA |
| msST2-27 | TATTGGTAGTGGGTTCAGAG | CTCTGAACCCACTACCAATA |
| msST2-28 | GTAGTGGGTTCAGAGCAGTA | TACTGCTCTGAACCCACTAC |
| msST2-29 | CAGAGCAGTAATAACTCTGC | GCAGAGTTATTACTGCTCTG |
| msST2-30 | GAGCAGTTATTAATTCCCTT | AAGGGAATTAATAACTGCTC |
| msST2-31 | GCAGTTATTAATTCCCTTGC | GCAAGGGAATTAATAACTGC |
| msST2-32 | ATTAATTCCCTTGCTTGGTC | GACCAAGCAAGGGAATTAAT |
| msST2-33 | ATCATGATAGATAGATGACC | GGTCATCTATCTATCATGAT |
| msST2-34 | AGATAGATGACCAACCTGAT | ATCAGGTTGGTCATCTATCT |
| msST2-35 | GATAGATGACCAACCTGATC | GATCAGGTTGGTCATCTATC |
| msST2-36 | ATAGATGACCAACCTGATCC | GGATCAGGTTGGTCATCTAT |
| msST2-37 | TAGATGACCAACCTGATCCG | CGGATCAGGTTGGTCATCTA |
| msST2-38 | AGATGACCAACCTGATCCGA | TCGGATCAGGTTGGTCATCT |
| msST2-39 | ATGACCAACCTGATCCGATT | AATCGGATCAGGTTGGTCAT |
| msST2-40 | ACCAACCTGATCCGATTTTA | TAAAATCGGATCAGGTTGGT |
| msST2-41 | CAACCTGATCCGATTTTAAA | TTTAAAATCGGATCAGGTTG |
| msST2-42 | AACCTGATCCGATTTTAAAA | TTTTAAAATCGGATCAGGTT |
| msST2-43 | ATCACTCTCTTATGATACTG | CAGTATCATAAGAGAGTGAT |
| msST2-44 | TCTTATGATACTGACCACTG | CAGTGGTCAGTATCATAAGA |
| msST2-45 | ACCACTGCCTTACTGGCAAT | ATTGCCAGTAAGGCAGTGGT |
| msST2-46 | CCACTGCCTTACTGGCAATA | TATTGCCAGTAAGGCAGTGG |
| msST2-47 | TGCCTTACTGGCAATACAAC | GTTGTATTGCCAGTAAGGCA |
| msST2-48 | TTACTGGCAATACAACTAAT | ATTAGTTGTATTGCCAGTAA |
| msST2-49 | TACTGGCAATACAACTAATA | TATTAGTTGTATTGCCAGTA |
| msST2-50 | CTGGCAATACAACTAATATG | CATATTAGTTGTATTGCCAG |
| msST2-51 | GAGACACTAACTCTAAGATC | GATCTTAGAGTTAGTGTCTC |
| msST2-52 | ACACTAACTCTAAGATCATA | TATGATCTTAGAGTTAGTGT |
| msST2-53 | AACTCTAAGATCATAGCAAT | ATTGCTATGATCTTAGAGTT |
| msST2-54 | ACTCTAAGATCATAGCAATA | TATTGCTATGATCTTAGAGT |
| msST2-55 | TATTGATTAAAAGGGTAGGG | CCCTACCCTTTTAATCAATA |
| msST2-56 | ATTAAAAGGGTAGGGTGGAG | CTCCACCCTACCCTTTTAAT |
| msST2-57 | GGGTAGGGTGGAGGTACCTA | TAGGTACCTCCACCCTACCC |
| msST2-58 | AGGGTGGAGGTACCTATTAG | CTAATAGGTACCTCCACCCT |
| msST2-59 | GGGTGGAGGTACCTATTAGA | TCTAATAGGTACCTCCACCC |
| msST2-60 | GGTGGAGGTACCTATTAGAT | ATCTAATAGGTACCTCCACC |
| msST2-61 | GTGGAGGTACCTATTAGATT | AATCTAATAGGTACCTCCAC |
| msST2-62 | TGGAGGTACCTATTAGATTC | GAATCTAATAGGTACCTCCA |
| msST2-63 | ACCTATTAGATTCAGTCATG | CATGACTGAATCTAATAGGT |
| msST2-64 | GATAATGGTGTTACTCCGGG | CCCGGAGTAACACCATTATC |
| msST2-65 | ATAATGGTGTTACTCCGGGT | ACCCGGAGTAACACCATTAT |
| msST2-66 | TAATGGTGTTACTCCGGGTG | CACCCGGAGTAACACCATTA |
| msST2-67 | AATGGTGTTACTCCGGGTGA | TCACCCGGAGTAACACCATT |
| msST2-68 | ATGGTGTTACTCCGGGTGAC | GTCACCCGGAGTAACACCAT |
| msST2-69 | GGTGTTACTCCGGGTGACTG | CAGTCACCCGGAGTAACACC |
| msST2-70 | TTACTCCGGGTGACTGTGGA | TCCACAGTCACCCGGAGTAA |
| msST2-71 | TACTCCGGGTGACTGTGGAC | GTCCACAGTCACCCGGAGTA |
| msST2-72 | ACTCCGGGTGACTGTGGACC | GGTCCACAGTCACCCGGAGT |
| msST2-73 | CGGGTGACTGTGGACCACAG | CTGTGGTCCACAGTCACCCG |
| msST2-74 | TGAGTGTACTTTAGTTCACG | CGTGAACTAAAGTACACTCA |
| msST2-75 | TCACGTTTGTATTAGAATTA | TAATTCTAATACAAACGTGA |
| msST2-76 | TTGAACAACACAGCGTATTA | TAATACGCTGTGTTGTTCAA |
| msST2-77 | AGCGTATTAAATAAAATTCG | CGAATTTTATTTAATACGCT |

1. A method for treating Alzheimer's Disease (AD) or reducing risk of Aβin a person in need thereof, comprising the step of administering to the person an effective amount of a composition disrupting a genomic sequence encompassing 3′-untranslated region (UTR) of sST2 gene/transcript.

2. The method of claim 1, further comprising, prior to the administering step, sequencing at least a portion of the person's genome.

3. The method of claim 1, wherein the person is an APOE-ε4 carrier or a non-APOE-ε4 carrier.

4. The method of claim 1, wherein the person is a female or a male.

5. (canceled)

6. The method of claim 1, wherein the person has been diagnosed with AD, or the person is not yet diagnosed with AD but has known risk factors for AD.

7. (canceled)

8. The method of claim 1, wherein the composition comprising an siRNA, a microRNA, a miniRNA, a lncRNA, or an antisense oligonucleotide targeting the 3′-UTR of the sST2 gene.

9. The method of claim 1, wherein the composition comprising one or more antisense oligonucleotides listed in Table 9.

10. The method of claim 9, wherein the composition comprises one vector encoding a Cas9 nuclease and two sgRNAs.

11. The method of claim 9, wherein the one or more vectors are one or more viral vectors.

12. The method of claim 1, wherein the composition is administered by subcutaneous, intramuscular, intravenous, intraperitoneal, or intracranial injection or by oral or nasal administration.

13. The method of claim 12, wherein the composition is administered in the form of a solution, a suspension, a powder, a paste, a tablet, or a capsule.

14. A kit for treating Alzheimer's Disease (AD) or reducing risk of Aβin a person in need thereof, comprising a container containing a composition disrupting a genomic sequence encompassing the 3′-UTR of the sST2 gene.

15. The kit of claim 14, wherein the composition is formulated for subcutaneous, intramuscular, intravenous, intraperitoneal, or intracranial injection, or for oral or nasal administration.

16. kit of claim 14, wherein the composition comprising an siRNA, a microRNA, a miniRNA, a lncRNA, or an antisense oligonucleotide targeting the genomic sequence encompassing the 3′-UTR of the sST2 gene.

17. The kit of claim 14, wherein the composition comprising one or more antisense oligonucleotides listed in Table 9.

18. The kit of claim 17, wherein the composition comprises one vector encoding a Cas9 nuclease and two sgRNAs.

19. The kit of claim 14, further comprising a second container containing agents for sequencing at least a portion of the person's genome.

20. The kit of claim 14, further comprising an instruction manual for administration of the composition.
